(19) United States
(12) Patent Application Publication
(10) Pub. No.: US 2021/0230693 A1
(43) Pub. Date: Jul. 29, 2021

(54) DIAGNOSIS AND PREDICTION OF AUTISM SPECTRUM DISORDER

(71) Applicant: LINEAGEN, INC., Salt Lake City, UT (US)

(72) Inventor: Charles H. Hensel, Salt Lake City, CA (US)

(21) Appl. No.: 16/951,470

(22) Filed: Nov. 18, 2020

Related U.S. Application Data
(63) Continuation of application No. 16/298,897, filed on Mar. 11, 2019, now abandoned.
Application No. 16/298,897 is a continuation of application No. 15/104,897, filed on Jun. 15, 2016, now abandoned.
(60) Provisional application No. 61/919,151, filed on Dec. 20, 2013.

(51) Int. Cl. C12Q 1/6883; G16B 20/00; G16B 25/00; G01N 33/483

(52) U.S. Cl. CPC C12Q 1/6883; G16B 20/00; C12Q 2600/156; G01N 33/483; G16B 25/00

(57) ABSTRACT

Methods and compositions for the detection of single nucleotide polymorphisms in a sample are provided. The methods and compositions are employed to determine whether the subject has autism spectrum disorder (ASD), is likely to develop ASD, or to classify a subject as having a particular ASD subtype. In one method of the invention, a sample is probed for one or more SNPs in Table 1, Table 2, Table 3, Table 6 or Table 7 at the nucleic acid level by performing a polymerase chain reaction (PCR) with primers specific to the SNPs. The presence and/or absence of the one or more SNPs is then compared to the presence and/or absence of the of the SNPs in at least one sample training set(s), where the comparing step comprises applying a statistical algorithm which comprises determining a correlation between the SNP data obtained from the sample and the SNP data from the at least one training set. The sample is diagnosed as ASD positive or ASD negative based on the results of the statistical algorithm.

### CROSS REFERENCE TO RELATED APPLICATION

This application is a continuation of U.S. application Ser. No. 16/298,897, filed Mar. 11, 20219, which is a continuation of U.S. application Ser. No. 15/104,897, filed Jun. 15, 2016, which is a U.S. National Stage Entry of International Application No. PCT/US2014/071984, filed Dec. 22, 2014, which application claims the benefit of priority from U.S. Provisional Application Ser. No. 61/919,151, filed Dec. 20, 2013, the disclosure of which is incorporated by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is LINE_007_02US_ST25.txt. The text file is 12 KB, was created on Mar. 11, 2019, and is being submitted electronically via EFS-Web.

### BACKGROUND OF THE INVENTION

Disorders of childhood development, also known as developmental delay disorders, are an ever growing group of disorders. Many disorders of childhood development are associated with aberrant copy number (i.e., gain or loss of copy number) of a particular subchromosomal region. According to the National Institute of Mental Health (NIMH), autism is included in a group of developmental brain disorders, collectively referred to as autism spectrum disorder (ASD). As the term “spectrum” suggests, ASD encompasses a wide range of symptoms, skills, and levels of impairment, or disability, that children with the disorder can have and is a complex, heterogeneous, behaviorally-defined disorder characterized by impairments in social interaction and communication as well as by repetitive and stereotyped behaviors and interests. The Diagnostic and Statistical Manual of Mental Disorders, Fourth Edition—Text Revision defines five disorders, sometimes called pervasive developmental disorders (PDDs), as ASD. These include: Autistic disorder (classic autism), Asperger's disorder (Asperger syndrome), Pervasive developmental disorder not otherwise specified (PDD-NOS), Rett's disorder (Rett syndrome), and Childhood disintegrative disorder (CDD).

The current state-of-the-art diagnosis of ASD is a series of various behavioral questionnaires. Because the ASD phenotype is so complicated, a molecular-based test would greatly improve the accuracy of diagnosis at an earlier age, when phenotypic/behavioral assessment is not possible, or integrated with phenotypic/behavioral assessment. Also, early diagnosis would allow initiation of ASD treatment at an earlier age which may be beneficial to short and long-term outcomes. Specifically, identification of genetic markers and biomarkers for ASD and other disorders of childhood development would allow identification of the disease, now typically diagnosed between ages three and five, in infancy or prenatal life.

Genetic evaluation of subjects suffering from childhood development disorder may also help predict out comes of both pharmacologic and behavioral therapies. Thus, there is an urgent need for a method of reliably identifying subjects with ASD or other disorders of childhood development. In particular there is need for a more accurate test for polymorphisms causing ASD and other childhood developmental delay disorders. Families with affected members would benefit from knowing whether they carry a mutation which could affect future pregnancies. Clinicians need a test as an aid in diagnosis, and researchers would use the test to classify subjects according to the etiology of their disease. The present invention addresses this and other needs.

Genetic factors play a substantial role in disorders of childhood development (Abrahams et al. (2008). Nat. Rev. Genet. 9, pp. 341-355; Matsunami et al. (2014). Molecular Autism 5, p. 5; Matsunami et al. (2013). PLOS one 8(1), p. e52239, the disclosure of each of which is incorporated by reference in their entireties for all purposes. Genetic mutations and chromosomal abnormalities that play a role in disorders of childhood development may be deletion or duplication variants, including copy number variants (CNV) or single nucleotide polymorphisms (SNPs). Previous genome-wide linkage and association studies have implicated multiple genetic regions that may be involved in autism and ASDs. Such heterogeneity increases the value of studies that include large extended pedigrees. Many autism studies have focused on small families (sibling pairs, or two parents and an affected offspring) to try to localize autism predisposition genes. These collections of small families may include cases with many different susceptibility loci. Subjects affected with ASD who are members of a large extended family may be more likely to share the same genetic causes through their common ancestors. Within such families, autism may be more genetically homogeneous.

### SUMMARY OF THE INVENTION

In one aspect, the present invention relates to a method for diagnosing a sample from a human subject as ASD-positive or ASD negative, and compositions for performing the method. In one embodiment, the method comprises detecting the presence of one or more SNP classifier biomarkers in Table 1, Table 2, Table 3, Table 6 or Table 7 at the nucleic acid level by a hybridization assay comprising the polymerase chain reaction (PCR) with primers specific to the classifier biomarkers; comparing the presence and/or absence of the one or more SNP classifier biomarkers of Table 1, Table 2, Table 3, Table 6 or Table 7 to the presence and/or absence of the of said SNP classifier biomarkers in at least one sample training set(s), wherein the at least one sample training set(s) comprise (i) data of the presence and/or absence of the one or more SNP classifier biomarkers of Table 1, Table 2, Table 3, Table 6 or Table 7 from an ASD positive sample or (ii) data of the presence and/or absence of the one or more SNP classifier biomarkers of Table 1, Table 2, Table 3, Table 6 or Table 7 from an ASD-negative sample. In one embodiment, the comparing step comprises applying a statistical algorithm which comprises determining a correlation between the SNP classifier biomarker data obtained from the sample and the SNP classifier biomarker data from the at least one training set. The sample is diagnosed as ASD positive or ASD negative based on the results of the statistical algorithm.

In another aspect, a method for classifying a sample from a human subject as a particular ASD subtype is provided. In one embodiment, the method comprises detecting the presence of one or more SNP classifier biomarkers in Table 1, Table 2, Table 3, Table 6 or Table 7 at the nucleic acid level by performing a hybridization assay comprising the polymerase chain reaction (PCR) with primers specific to the classifier biomarkers; comparing the presence and/or absence of the one or more SNP classifier biomarkers of Table 1, Table 2, Table 3, Table 6 or Table 7 to the presence and/or absence of the of said SNP classifier biomarkers in at least one sample training set(s). The at least one sample training set(s) comprises (i) data of the presence and/or absence of the one or more SNP classifier biomarkers of Table 1, Table 2, Table 3, Table 6 or Table 7 from a first ASD subtype positive sample or (ii) data of the presence and/or absence of the one or more SNP classifier biomarkers of Table 1, Table 2, Table 3, Table 6 or Table 7 from a second ASD subtype-positive sample. The comparing step comprises applying a statistical algorithm which comprises determining a correlation between the SNP classifier biomarker data obtained from the sample and the SNP classifier biomarker data from the at least one training set. The sample is diagnosed as a particular ASD subtype based on the results of the statistical algorithm.

In a further embodiment, the first ASD subtype and second ASD subtype are selected from the group consisting of Autistic disorder (classic autism), Asperger's disorder (Asperger syndrome), Pervasive developmental disorder not otherwise specified (PDD-NOS), and Childhood disintegrative disorder (CDD), wherein the first ASD subtype and second ASD subtype are different.

In one embodiment, with respect to the above aspects, the one or more SNP classifier biomarkers comprises two or more SNP classifier biomarkers, three or more SNP classifier biomarkers, four or more SNP classifier biomarkers, five or more SNP classifier biomarkers, six or more SNP classifier biomarkers, seven or more SNP classifier biomarkers, eight or more SNP classifier biomarkers, nine or more SNP classifier biomarkers, ten or more SNP classifier biomarkers, eleven or more SNP classifier biomarkers, twelve or more SNP classifier biomarkers, thirteen or more SNP classifier biomarkers, fourteen or more SNP classifier biomarkers, fifteen or more SNP classifier biomarkers, twenty or more SNP classifier biomarkers, twenty-five or more SNP classifier biomarkers, or thirty or more SNP classifier biomarkers from Table 1, 2, 3, 6 or 7.

The hybridization assay, in one embodiment, is a microarray assay, a high throughput sequencing assay, a quantitative PCR assay, or a combination thereof. The sample from the human subject, in one embodiment, is a buccal sample.

In one embodiment, the methods and compositions provided herein detect an SNP in each of the RAB11FIP5, ABP1, and JMJD7-PLA2G4B genes. In a further embodiment, the RAB11FIP5 SNP is located at chr2: 73302656 (hg19), the ABP1 SNP is located at chr7:150554592 (hg19) and the JMJD7-PLA2G4B SNP is located at chr15:42133295 (hg19).

In one aspect, the methods provided herein can further comprise identifying a human subject for ASD therapy based on the results of the statistical algorithm.

### BRIEF DESCRIPTION OF THE FIGURES

FIG. 1: Workflow for sequence variant discovery and analysis. Only ethnicity and gender matched, unrelated, cases and controls were used for association testing.

FIG. 2: Co-segregation of a RAB11FIP5 variant. Two generation pedigree (Pedigree 1) with three male siblings affected with autism. Sequence variants identified in the family are shown in the black boxes. Open boxes—unaffected male family members; open circles—unaffected female family members; filled boxes—affected male family members. Odds ratios for the variants observed in the case/control study are shown. Variants with no odds ratio were observed only in high-risk families. All family members were tested for all variants.

FIG. 3: Segregation of C14orf2 variant. Two generation pedigree (Pedigree 2), with three affected female and two affected male siblings as well as an affected male half-sibling. The C14ORF2 variant segregates to five of six affected children. Pedigree symbols are described in the legend for FIG. 2. Sequence variants identified in the family are shown in the black boxes. A CNV found in the affected half-sibling [27] is shown in the red box. Odds ratios for variants observed in the case/control study are shown in parentheses. Variants with no odds ratio were observed only in high-risk families. All family members were tested for all variants unless no DNA was available. Individuals with no available DNA are indicated.

FIG. 4: Segregation of KLHL6, SPATA5L1, and ITPK1 variants. Two generation pedigree (Pedigree 3), with five affected male siblings. Sequence variants identified in the family are shown in the black boxes. Pedigree symbols are described in the legend for FIG. 2. Variants with no odds ratio were observed only in high-risk families. All family members were tested for all variants.

FIG. 5: Segregation of DEFB124 variant in a multigeneration pedigree. Pedigree 4 has seven children affected with autism. Links between this pedigree and other high-risk autism pedigrees are indicated by blue boxes. Sequence variants identified in the family are shown in the black boxes. CNVs inherited by two individuals [27] are shown in red boxes. Pedigree symbols are described in the legend for FIG. 2. Odds ratios for the variants observed in the case/control study are shown in parentheses. Variants with no odds ratio were observed only in high-risk families. All family members were tested for all variants unless no DNA was available. Individuals with no available DNA are indicated.

FIG. 6: Segregation of multiple variants including a sequence variant in AKAP9 and a copy number variant in NRXN1 in a multi-generation pedigree. Pedigree 5 has nine children affected with autism. A link between this pedigree and another high-risk autism pedigree is indicated by the blue box. Sequence variants identified in the family are shown in the black boxes. CNVs identified in 4 individuals [27] are shown in red boxes. Pedigree symbols are described in the legend for FIG. 2. Odds ratios for the variants observed in the case/control study are shown in parentheses. Variants with no odds ratio were observed only in high-risk families. All family members were tested for all variants unless no DNA was available. Individuals with no available DNA are indicated.

FIG. 7A-FIG. 7C. Haplotype sharing in high-risk autism pedigrees. The figures show a graphic representation of haplotype sharing among affected individuals in a pedigree, created using the HapShare program. The X-axis represents chromosomal coordinates for the designated chromosomes. The Y-axis represents various combinations of haplotype sharing among affected individuals in the pedigree, listed arbitrarily by iteration number. The lowest value on the Y-axis represent sharing among all N affected individuals in the pedigree, and where all N individuals share, there is only one possible combination. With lower degrees of sharing there are more possibilities. For example, in pedigree 10 with 6 affected individuals, there is only one possible way for all 6 to share the same haplotype. Where only 5 of 6 share the haplotype, there are 6 different ways to get this result, with each of the 6 affected individuals being excluded from sharing in each of the 6 iterations shown. With lower degrees of sharing there are more possibilities, and each possibility is shown as a separate row on the Y-axis. Shared regions are indicated by the colored blocks. Red indicates sharing among N out of N affected individuals in the pedigree, with other colors representing lower degrees of sharing. In FIG. 7A two regions of chromosome 2 shared by all 6 affected individuals in pedigree 10; FIG. 7B sharing among all 6 affected individuals in pedigree 10 of a chromosome 14 region; FIG. 7C sharing among 5 of 8 affected individuals on chromosome 7 in pedigree 5 and sharing among 4 of 7 affected individuals on chromosome 20 in pedigree 4. The variants found on these haplotypes, if any, are indicated by the gene names in the figure. Note that the chromosome 7 region identified in pedigree 5 as being shared among 8 affected individuals was later shown not to be shared by an additional affected family member, resulting in a final count of sharing among 5 of 9 affected individuals.

FIG. 8. SNP genotype clusters. Genotype clusters for all SNPs observed in the case/control study (Table 3) are shown.

FIG. 9. Sanger sequence confirmation of variants in the RAB11FIP5, AUP1, SCN3A, ATP11B, KLHL6, C7orf10, AKAP9, HEPACAM2, PDK4, RELN, ABP1, ALX1, AP1G2, DCAF11, RNF31, IRF9, SDR39U1 and PRKD1 genes. Heterozygous positions are indicated by the blue line in the center of each panel.

FIG. 10. Sanger sequence confirmation of variants in the SEC23A, ITPK1, CLMN, CCDCl85C, MOK, C14orf2, TRPM1, FMN1, PGBD4, OIP5, JMJD7, JMJD7-PLA2G4B, CASC4, SPATA5L1, PYGO1, PRTG, NUDT7, DEFB124, and EPB41L1 genes. Heterozygous positions are indicated by the blue line in the center of each panel.

FIG. 11. Segregation of a second AKAP9 variant in a small pedigree. Pedigree 6 has a single affected child. Pedigree symbols are described in the legend for FIG. 2. A link between this pedigree and other high-risk autism pedigrees is indicated by blue boxes. Sequence variants identified in the family are shown in the black boxes. Odds ratios for the variants observed in the case/control study are shown in parentheses. Variants with no odds ratio were observed only in high-risk families. All family members were tested for all variants unless no DNA was available. Individuals with no available DNA are indicated.

FIG. 12. Segregation of an ALX1 variant in a small two-generation pedigree. Pedigree 6 has two siblings affected with autism. A single ALX1 variant is shared by both siblings. A link between this pedigree and another high-risk autism pedigree is indicated by the blue box. Pedigree symbols are described in the legend for FIG. 2. Sequence variants identified in the family are shown in the black boxes. Odds ratios for the variants observed in the case/control study are shown in parentheses. Variants with no odds ratio were observed only in high-risk families. All family members were tested for all variants.

FIG. 13. Multigeneration pedigree with multiple sequence variants and overlapping loss and gain copy number variants. Pedigree 8 has 5 affected male children. Potential causal variants in this family do not segregate to more than one affected individual. CNVs identified in 4 individuals [27] are shown in red boxes. Pedigree symbols are described in the legend for FIG. 2. Sequence variants identified in the family are shown in the black boxes. Odds ratios for the variants observed in the case/control study are shown in parentheses. Variants with no odds ratio were observed only in high-risk families. All family members were tested for all variants unless no DNA was available. Individuals with no available DNA are indicated.

FIG. 14. Segregation of two sequence variants in a two generation pedigree. Pedigree nine has three affected female siblings. Pedigree symbols are described in the legend for FIG. 2. Sequence variants identified in the family are shown in the black boxes. All family members were tested for all variants.

FIG. 15. Segregation of sequence variants in SCN3A and 01P5 and CNVs involving LINGO2 in pedigree 10. Pedigree 10 has 6 affected male siblings. The female sibling in the lowest generation has trisomy 21 and includes some features of autism. The LINGO2 loss CNV was shown to have an odds ratio of 3.74 in our case/control study, while the LINGO2 gain CNV did not have a clinically relevant odds ratio in the broad ASD population. The SCN3A sequence variant was not observed in our case/control study while the 01P5 variant yielded an odds ratio of 2.25. Pedigree symbols are described in the legend for FIG. 2. Sequence variants identified in the family are shown in the black boxes. All family members with DNA available were tested for all variants.

FIG. 16A-FIG. 1611. Effects of RAB11FIP5 P652L on RAB11 binding. (FIG. 16A) Wild type of P652L mutant FIP5(490-653) was incubated with either various GST-tagged Rabs or GST-tagged FIPs. Beads were then washed and bound FIP5(490-653) eluted with 1% SDS. Eluates were then analyzed by immunoblotting with anti-Rab11FIP5 antibodies. FIG. 16B-FIG. 16G HeLa cells were transduced with either wild type FIP5-GFP (FIG. 16A and FIG. 16D) or FIP5-GFP-P652L (FIG. 16E and FIG. 16G). Cells were then fixed and stained with anti-transferrin receptor antibodies (FIG. 16C, FIG. 16D, FIG. 16F and FIG. 16G). FIG. 16D and FIG. 16E are merged images, with yellow representing the extent of overlap between Rab11FIP5 and transferrin receptor. (FIG. 16H) HeLa cells expressing either FIP5-GFP or FIP5-GFP-P652L were incubated with 1 μg/ml of transferrin-Alexa488. Cells were then washed and incubated in serum-supplemented media varying amount of time. Cell-associated (not recycled) transferrin-Alexa488 was measured using flow cytometry. Data shown are the means of two independent experiments.

### DETAILED DESCRIPTION OF THE INVENTION

When the human genomes of two individuals are compared, they are 99.9% identical (Kwok and Chen (2003). Curr. Issues Mol. Biol. 5, pp. 43-60, incorporated by reference in its entirety). However, because the human genome is approximately 3.2 billion base pairs in size, there are about 3.2 million base pair differences from one genome to another. Most of the differences are attributed to single base substitution polymorphisms, popularly known as single nucleotide polymorphisms (SNPs). (Kwok and Chen (2003). Curr. Issues Mol. Biol. 5, pp. 43-60). A fraction of the polymorphisms have functional significance and are thought to be the basis for the diversity found among humans (Collins et al. (1997). Science 278, pp. 1580-1581, incorporated by reference in its entirety). In the case of the present invention, samples are obtained from subjects and particular SNPs are analyzed in order to assess whether the subject is at risk for developing autism spectrum disorder (ASD) or to diagnose the subject with an ASD.

In some aspects, the methods provided herein are directed to (i) diagnosing a subject with an ASD, (ii) predicting whether a subject is at risk for an ASD or assess the likelihood of the subject for developing ASD, e.g., autism, (iii) diagnosing a subject with a particular ASD subtype, or (iv) selecting a subject for the treatment of ASD. The methods comprise in part determining the presence of one or more SNPs in one or more of the following genes, for example, SNPs at the positions provided in Table 1: RAB11FIP5, AUP1, SCN3A, ATP11B, KLHL6, C7orf10, AKAP9, HEPACAM2, PDK4, RELN, ABP1, ALX1, AP1G2, DCAF11, RNF31, IRF9, SDR39U1, PRKD1, SEC23A, ITPK1, CLMN, CCDC85C, MOK, C14orf2, TRPM1, FMN1, PGBD4, OIP5, JMJD7-PLA2G4B, CASC4, SPATA5L, PYGO1, PRTG, NUDT7, DEFB124, EPB41L1. In a further embodiment, the presence or absence of two or more SNPs of the aforementioned genes is determined. In even a further embodiment, the presence or absence of five or more SNPs of the aforementioned genes is determined. In even a further embodiment, the presence or absence of ten or more SNPs of the aforementioned genes is determined.

In the context of the present invention, reference to “one or more,” “two or more,” “five or more,” etc. of the SNPs listed in any particular SNP set means any one or any and all combinations of the SNPs listed.

In one embodiment, the methods and compositions provided herein detect an SNP in each of the RAB11FIP5, ABP1, and JMJD7-PLA2G4B genes. In a further embodiment, the RAB11FIP5 SNP is located at chr2: 73302656 (hg19), the ABP1 SNP is located at chr7:150554592 (hg19) and the JMJD7-PLA2G4B SNP is located at chr15:42133295 (hg19).

In one embodiment, the one or more SNPs comprises one or more, two or more, three or more, four or more, five or more, 10 or more, 15 or more, 20 or more, 25 or more, 30 or more or 35 or more SNPs in the genes provided above, for example SNPs in Table 1, 2, 3, 6 or 7, for example one or more SNPs in the RAB11FIP5, ABP1, and JMJD7-PLA2G4B genes. In a further embodiment, the one or more (e.g., two or more, or five or more) SNPs detectable with the methods and compositions provided herein can be combined with other markers for the diagnosis of ASD, the prediction of ASD in a subject, the diagnosis of a particular ASD subtype. For example, one or more (e.g., two or more, or five or more) of the single nucleotide polymorphisms (e.g., two or more, or five or more) associated with ASD disclosed in U.S. Patent Application Publication No. 2010/0210471, incorporated by reference in its entirety for all purposes, and International PCT publication no. 2014/055915, incorporated by reference in its entirety for all purposes, can be detected in combination with the one or more SNPs described herein in one or more of the compositions or methods. Additionally, one or more of the CNVs (e.g., two or more, or five or more) associated with ASD disclosed in U.S. Patent Application Publication No. 2010/0210471, incorporated by reference in its entirety for all purposes, and/or one or more of the CNVs (e.g., two or more, or five or more) described in International PCT publication no. 2014/055915, incorporated by reference in its entirety for all purposes, can be detected in combination with the SNPs described herein in one or more of the compositions or methods.

Accordingly, aspects of the present invention relate to methods and compositions for the detection of one or more SNPs in a subject to either (i) diagnosing a subject with an ASD, (ii) predicting whether a subject is at risk for an ASD or assess the likelihood of the subject for developing ASD, e.g., autism, (iii) diagnosing a subject with a particular ASD subtype, or (iv) selecting a subject for the treatment of ASD. In one embodiment of these aspects, a sample obtained from a human subject and is analyzed for the presence of one or more of the SNPs set forth in Table 1, 2, 3, 6 or 7. The results are then compared to reference values, and depending on the comparison, the subject is diagnosed with an ASD, is predicted to be at risk for an ASD, a particular ASD subtype is diagnosed or the subject is selected for treatment of ASD. In one embodiment, the ASD subtype is autistic disorder.

The Diagnostic and Statistical Manual of Mental Disorders, Fourth Edition—Text Revision currently defines five disorders (also referred to herein as “ASD subtypes”), sometimes called pervasive developmental disorders (PDDs), as ASD. These include: Autistic disorder (classic autism), Asperger's disorder (Asperger syndrome (AS)), Pervasive developmental disorder not otherwise specified (PDD-NOS), Rett's disorder (Rett syndrome), and Childhood disintegrative disorder (CDD). It is noted that the majority of Rett syndrome cases are known to be caused by mutations in either the MeCP2 gene or the CDKL5 gene and it is anticipated that updated revisions of the Diagnostic and Statistical Manual of Mental Disorders will classify Rett syndrome separately from ASD. Therefore, in certain embodiments, ASD does not include Rett syndrome. Autistic disorder is understood as any condition of impaired social interaction and communication with restricted repetitive and stereotyped patterns of behavior, interests and activities present before the age of 3, to the extent that health may be impaired. Asperger syndrome is distinguished from autistic disorder by the lack of a clinically significant delay in language development in the presence of the impaired social interaction and restricted repetitive behaviors, interests, and activities that characterize ASD. PDD-NOS is used to categorize individuals who do not meet the strict criteria for autism but who come close, either by manifesting atypical autism or by nearly meeting the diagnostic criteria in two or three of the key areas. The methods and compositions provided herein are amenable for use to diagnose a subject with any of the disorders on the ASD spectrum, or to predict whether a subject will develop any of the disorders on the ASD spectrum.

A “single nucleotide polymorphism (SNP)” is a single basepair variation in a nucleic acid sequence. Polymorphisms can be referred to, for instance, by the nucleotide position at which the variation exists, by the change in amino acid sequence caused by the nucleotide variation, or by a change in some other characteristic of the nucleic acid molecule that is linked to the variation (e.g., an alteration of a secondary structure such as a stem-loop, or an alteration of the binding affinity of the nucleic acid for associated molecules, such as polymerases, RNases, and so forth). By way of example, the SNP disclosed herein in the region of the genes set forth herein can be referred to by its location in the respective gene or chromosome, e.g., based on the numerical position of the variant residue or chromosome position. SNPs detectable by the methods and compositions provided in Tables 1, 2, 3, 6 and 7. In another embodiment, any SNP at the chromosome locations provided in Table 1 are used in the methods described herein and detectable with the compositions provided herein.

TABLE 1
Position of SNPs detectable with the methods
and compositions described herein.

| Gene | Chr: Position (hg19) |
|---|---|
| RAB11FIP5 | chr2: 73302656 |
| RAB11FIP5 | chr2: 73302656 |
| AUP1 | chr2: 74756328 |
| SCN3A | chr2: 165946964 |
| ATP11B | chr3: 182583394 |
| KLHL6 | chr3: 183226296 |
| C7orf10 | chr7: 40498796 |
| AKAP9 | chr7: 91724455 |
| AKAP9 | chr7: 91736684 |
| HEPACAM2 | chr7: 92825188 |
| PDK4 | chr7: 95215047 |
| RELN | chr7: 103214555 |
| ABP1 | chr7: 150554592 |
| ALX1 | chr12: 85674230 |
| AP1G2 | chr14: 24035159 |
| DCAF11 | chr14: 24590630 |
| RNF31 | chr14: 24617687 |
| IRF9 | chr14: 24634003 |
| SDR39U1 | chr14: 24909513 |
| PRKD1 | chr14: 30095731 |
| SEC23A | chr14: 39545251 |
| ITPK1 | chr14: 93418316 |
| CLMN | chr14: 95679692 |
| CCDC85C | chr14: 99988547 |
| MOK | chr14: 102749873 |
| C14orf2 | chr14: 104381450 |
| TRPM1 | chr15: 31329966 |
| FMN1 | chr15: 33359761 |
| PGBD4 | chr15: 34395847 |
| OIP5 | chr15: 41611874 |
| JMJD7 | chr15: 42129054 |
| JMJD7-PLA2G4B | chr15: 42133295 |
| CASC4 | chr15: 44620915 |
| SPATA5L1 | chr15: 45695534 |
| PYGO1 | chr15: 55839207 |
| PRTG | chr15: 55916638 |
| NUDT7 | chr16: 77756514 |
| DEFB124 | chr20: 30053379 |
| EPB41L1 | chr20: 34809850 |

TABLE 2

| | Chr: | Reference | Variant |
|---|---|---|---|
| Gene | Position (hg19) | Allele (+) | Allele (+) |
| RAB11FIP5 | chr2: 73302656 | G | A |
| RAB11FIP5 | chr2: 73302656 | G | T |
| AUP1 | chr2: 74756328 | C | G |
| SCN3A | chr2: 165946964 | T | C |
| ATP11B | chr3: 182583394 | T | C |
| KLHL6 | chr3: 183226296 | A | G |
| C7orf10 | chr7: 40498796 | C | T |
| AKAP9 | chr7: 91724455 | C | T |
| AKAP9 | chr7: 91736684 | C | T |
| HEPACAM2 | chr7: 92825188 | C | T |
| PDK4 | chr7: 95215047 | G | C |
| RELN | chr7: 103214555 | C | G |
| ABP1 | chr7: 150554592 | G | C |
| ALX1 | chr12: 85674230 | G | T |
| AP1G2 | chr14: 24035159 | G | A |
| DCAF11 | chr14: 24590630 | G | A |
| RNF31 | chr14: 24617687 | G | A |
| IRF9 | chr14: 24634003 | G | C |
| SDR39U1 | chr14: 24909513 | G | A |
| PRKD1 | chr14: 30095731 | T | A |
| SEC23A | chr14: 39545251 | C | T |
| ITPK1 | chr14: 93418316 | G | A |
| CLMN | chr14: 95679692 | G | C |
| CCDC85C | chr14: 99988547 | G | A |
| MOK | chr14: 102749873 | G | A |
| C14orf2 | chr14: 104381450 | A | G |
| TRPM1 | chr15: 31329966 | G | T |
| FMN1 | chr15: 33359761 | T | C |
| PGBD4 | chr15: 34395847 | G | T |
| OIP5 | chr15: 41611874 | G | A |
| JMJD7 | chr15: 42129054 | C | T |
| JMJD7-PLA2G4B | chr15: 42133295 | T | A |
| CASC4 | chr15: 44620915 | C | T |
| SPATA5L1 | chr15: 45695534 | G | C |
| PYGO1 | chr15: 55839207 | C | G |
| PRTG | chr15: 55916638 | C | G |
| NUDT7 | chr16: 77756514 | G | A |
| DEFB124 | chr20: 30053379 | G | A |
| EPB41L1 | chr20: 34809850 | A | G |

TABLE 3

| | | Refer- | | | | | | |
|---|---|---|---|---|---|---|---|---|
| | Chr: | ence | Variant | Forward | SEQ | Reverse | SEQ | |
| | Position | Allele | Allele | Primer | ID | Primer | ID | Amplicon |
| Gene | (hg19) | (+) | (+) | Sequence | No. | Sequence | No. | (hg19) |
| RAB11FIP5 | chr2:73302656 | G | A | GTGACAAGGC | 1 | TGAGCTCATC | 2 | chr2:73302539- |
| | | | | AAGACAGACG | | AGCCTGCTC | | 73302802 |
| RAB11FIPS | chr2:73302656 | G | T | GTGACAAGGC | 3 | TGAGCTCATC | 4 | chr2:73302539- |
| | | | | AAGACAGACG | | AGCCTGCTC | | 73302802 |
| AUP1 | chr2:74756328 | C | G | GGCCTCGCTC | 5 | GGACTCCGG | 6 | chr2:74756241- |
| | | | | TCACTCAC | | GATCACAGT | | 74756351 |
| SCN3A | chr2:165946964 | T | C | TCCTCCCTTT | 7 | CAACCACTT | 8 | chr2:165946857- |
| | | | | AATTGCCTCT | | TGAAACGTA | | 165946988 |
| | | | | T | | AACAA | | |
| ATP11B | chr3:182583394 | T | C | GATGCAGTTT | 9 | TGGTTCTGA | 10 | chr3:182583291- |
| | | | | CGGGAATGTT | | AAGAGGAAC | | 182583463 |
| | | | | | | TGG | | |
| KLHL6 | chr3:1832262% | A | G | ATTCCAACGC | 11 | CCTCCTTGT | 12 | chr3:183226226- |
| | | | | AGTTTTCTGG | | GGACTCACC | | 183226352 |
| | | | | | | AT | | |
| C7orf10 | chr7:404987% | C | T | CCAGCAAGGA | 13 | TCTCTCCAC | 14 | chr7:40498589- |
| | | | | ATGTTCTTGA | | CAGCCAGTT | | 40498938 |
| | | | | G | | TT | | |
| AKAP9 | chr7:91724455 | C | T | TGGGCTTTGG | 15 | TGACATTTT | 16 | Chr7:91724422- |
| | | | | AGAAAGAGAA | | AGATGGAGG | | 91724571 |
| | | | | | | AAAGC | | |
| AKAP9 | chr7:91736684 | C | T | CTTCTGGTGG | 17 | ATTCCAGGC | 18 | chr7:91736637- |
| | | | | GCTGGAGTTA | | AGGTTTTCT | | 91736765 |
| | | | | | | CA | | |
| HEPACAM2 | chr7:92825188 | C | T | CACACTGCCC | 19 | ATTTCAGGC | 20 | chr7.92825051- |
| | | | | AGTGCTTAAA | | CATGAAGAT | | 92825221 |
| | | | | | | GC | | |
| PDK4 | Chr7:95215047 | G | C | CACCAGTCAT | 21 | AAGTGCAAA | 22 | chr7:95215002- |
| | | | | CAGCCTCAGA | | TTATGCCAT | | 95215155 |
| | | | | | | GC | | |
| RELN | chr7:103214555 | C | G | CTTGTTACCT | 23 | AAGCTCAGC | 24 | chr7:103214531- |
| | | | | GATATTCCTG | | CCTCTGTGG | | 103214681 |
| | | | | GTG | | TA | | |
| ABP1 | chr7:150554592 | G | C | GCAACGCTGT | 25 | GGAAAGTGT | 26 | Chr7:150554499- |
| | | | | GCTCTACG | | CCAGGAAGG | | 150554762 |
| | | | | | | TG | | |
| ALX1 | chr12:85674230 | G | T | GGAGACGCTG | 27 | CTAGCGACT | 28 | chr12:85674135- |
| | | | | GACAATGAGT | | CACCGCTGC | | 85674277 |
| | | | | | | T | | |
| AP1G2 | chr!4:24035159 | G | A | GTCGGGGAAG | 29 | CGTCACCAT | 30 | chr14:24035U3- |
| | | | | TGAATGGTG | | GGTAAGGCT | | 24035282 |
| | | | | | | GT | | |
| DCAF11 | chr14:24590630 | G | A | GGTTTACTCT | 31 | CAGTGGAGC | 32 | chr14:24590550- |
| | | | | GCATCCCTAC | | AGCCACTGT | | 24590714 |
| | | | | CC | | AG | | |
| RNF31 | chr14:24617687 | G | A | CTTGATGGAC | 33 | ACAAAGCCC | 34 | chr14:24617641- |
| | | | | TTATGCACCA | | TCCCTCTAA | | 24617806 |
| | | | | | | GC | | |
| IRF9 | chr14:24634003 | G | C | GAGCAGCATG | 35 | GGTTGCTGG | 36 | chr14:24633926- |
| | | | | GAGCAGGT | | CCACTAGGA | | 24634026 |
| | | | | | | T | | |
| SDR39U1 | chr14:24909513 | G | A | GTCTGGGCAA | 37 | CTTCCCCTG | 38 | chr14:24909479- |
| | | | | ACTCAGCATT | | GATACACAT | | 24909604 |
| | | | | | | CG | | |
| PRKD1 | chr14:30095731 | T | A | TGTTTTTCCTGT | 39 | CATTGGGCTTGT | 40 | chr14:30095608- |
| | | | | AAATATCGCTTT | | ACCTCTAGGA | | 30095782 |
| SEC23A | chr14:39545251 | C | T | ATCTCCAACCA | 41 | TTCATATGTTTT | 42 | chr14:39545214- |
| | | | | CCATTCCAG | | CTTTTAAACTCT | | 39545339 |
| | | | | | | TGA | | |
| ITPK1 | chr14:93418316 | G | A | CTACCCTGCTG | 43 | CCTTCCTGTCGC | 44 | chr14:93418185- |
| | | | | GAGAGCTTG | | TTTTTCAG | | 93418378 |
| CLMN | chr14:95679692 | G | C | GGCCTTGATAG | 45 | GGCAACCTCAGC | 46 | chr14:95679588- |
| | | | | CCTTCCTCT | | AGAAACTC | | 95679731 |
| CCDC85C | chr14:99988547 | G | A | CTCACGTTCTG | 47 | CCCTCCGTCTAA | 48 | chr14:99988465- |
| | | | | CAGGGAGTC | | CCCCTCT | | 99988613 |
| MOK | chr14:102749873 | G | A | GCTGCTTCATT | 49 | AAAGTTTGCTGT | 50 | chr14:102749825- |
| | | | | TGTTTACATGC | | CTGGAAGTG | | 102750005 |
| | | | | | | A | | |
| C14orf2 | chr14:104381450 | A | G | TTCCTGACCTC | 51 | CCCCATGAAGCC | 52 | chr14:104381365- |
| | | | | AGAAAAATCAAA | | CTACTACA | | 104381494 |
| TRPM1 | chr15:31329966 | G | T | AAGCCCTTGAA | 53 | TGTGCTGTGCTC | 54 | chr15:31329831- |
| | | | | GTTTTTCTTGA | | TGTTTTCC | | 31330067 |
| FMN1 | chr15:33359761 | T | C | CAGAATCACTG | 55 | ACCTGACCTCGG | 56 | chr15:33359641- |
| | | | | GTGGTGTGC | | AAATGATG | | 33359822 |
| PGBD4 | Chr15:34395847 | G | T | GACTGATGCAG | 57 | CAACATTGTCAC | 58 | chr15:34395773- |
| | | | | TTGGGACAG | | CTCCTTGC | | 34395920 |
| OIP5 | chr15:41611874 | G | A | AATTTATTTGA | 59 | TCTGTGGTTCTT | 60 | chr15:41611781- |
| | | | | TGGACTTTGTC | | GTGGGATTC | | 41611961 |
| | | | | TCAA | | | | |
| JMJD7 | chr15:42129054 | C | T | GGGACAGAGCC | 61 | ACGTGGTGGAAC | 62 | chr15:42128915- |
| | | | | TGAAGTCCT | | CACAGAG | | 42129112 |
| JMJD7- | chr15:42133295 | T | A | TGCACTCCTTC | 63 | AGTGCTGTCCTT | 64 | chr15:42133206- |
| PLA2G4B | | | | TGACCCTTT | | CCCACAAG | | 42133350 |
| CASC4 | chr15:44620915 | C | T | CATCCCATAGC | 65 | TTCACAAGGTAA | 66 | chr15:44620808- |
| | | | | TTCTGAATAGGA | | GTATTGTTC | | 44620967 |
| | | | | | | TTCC | | |
| SPATA5L1 | chr15:45695534 | G | C | GGAGACCGAGG | 67 | GTCAACACCTGG | 68 | chr15:45695455- |
| | | | | AGAACGTG | | GCCACTAC | | 45695607 |
| PYGO1 | chr15:55839207 | C | G | ATAGCCTCCAA | 69 | CACCACCGAATC | 70 | chr15:55839178- |
| | | | | AGCCAGGAT | | CAAACTCT | | 55839311 |
| PRTG | chr15:55916638 | C | G | GCTCCTTCCAG | 71 | TGATAGGCCAGG | 72 | chr15:55916601- |
| | | | | GTTCTTTCC | | TGGTTCAT | | 55916779 |
| NTJDT7 | chr16:77756514 | G | A | CTTTAGGCCGC | 73 | GCCTCCGCTACG | 74 | chr16:77756384- |
| | | | | TCCCAAG | | ATCAAG | | 77756579 |
| DEFBI24 | chr20:30053379 | G | A | GGACAGCAGGA | 75 | CCTGCCAAACTT | 76 | chr20:30053293- |
| | | | | ACCAGCTAC | | ACTGCACA | | 30053427 |
| EPB41L1 | chr20:34809850 | A | G | GTGACCTCACC | 77 | ACAGGGTCAGCA | 78 | chr20:34809758- |
| | | | | TCCCTCTCC | | AGAAGTGG | | 34809989 |

“Sample” or “biological sample,” as used herein, refers to a sample obtained from a human subject or a patient, which may be tested for a particular molecule, for example one or more of the single nucleotide polymorphisms (SNPs) or copy number variants (CNV) set forth herein, such as a one or more of the SNPs set forth in Tables 1, 2, 3, 6 or 7. Samples may include but are not limited to cells, buccal swab sample, body fluids, including blood, serum, plasma, urine, saliva, cerebral spinal fluid, tears, pleural fluid and the like.

Samples that are suitable for use in the methods described herein contain genetic material, e.g., genomic DNA (gDNA). Non-limiting examples of sources of samples include urine, blood, and tissue. The sample itself will typically consist of nucleated cells (e.g., blood or buccal cells), tissue, etc., removed from the subject. The subject can be an adult, child, fetus, or embryo. In some embodiments, the sample is obtained prenatally, either from a fetus or embryo or from the mother (e.g., from fetal or embryonic cells in the maternal circulation). Methods and reagents are known in the art for obtaining, processing, and analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a mouthwash sample.

The sample may be further processed before the detecting step. For example, DNA in a cell or tissue sample can be separated from other components of the sample. The sample can be concentrated and/or purified to isolate DNA. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., genomic DNA. All samples obtained from a subject, including those subjected to any sort of further processing, are considered to be obtained from the subject.

Once a sample is obtained, it is interrogated for one or more of the SNPs set forth herein, e.g., one or more of the SNPs set forth in Tables 1, 2, 3, 6 or 7.

In general, the one or more of the SNPs can be identified using an oligonucleotide hybridization assay alone or in combination with an amplification assay, i.e., to amplify the nucleic acid in the sample prior to detection. In one embodiment, the genomic DNA of the sample is sequenced or hybridized to an array, as described in detail below. A determination is then made as to whether the sample includes the one or more SNPs or rather, includes the “normal” or “wild type” sequence (also referred to as a “reference sequence” or “reference allele”). In the case of the SNPs described herein, in one embodiment, the “reference allele” is provided in Table 2

In general, if the hybridization assay reveals a difference between the sequenced region and the reference sequence, a polymorphism has been identified. Certain statistical algorithms can aid in this determination, as described herein. The fact that a difference in nucleotide sequence is identified at a particular site that determines that a polymorphism exists at that site. In most instances, particularly in the case of SNPs, up to four variants may exist since there are four naturally occurring nucleotides in DNA.

For example, an oligonucleotide or oligonucleotide pair can be used in methods known in the art, for example in a microarray or polymerase chain reaction assay, to detect the one or more SNPs.

The term “oligonucleotide” refers to a relatively short polynucleotide (e.g., 100, 50, 20 or fewer nucleotides) including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

In the context of the present invention, an “isolated” or “purified” nucleic acid molecule, e.g., a DNA molecule or RNA molecule, is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule is substantially free of other cellular material or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

As used herein a set of oligonucleotides may comprise from about 2 to about 100 oligonucleotides, all of which specifically hybridize to a particular genetic marker (which includes an SNP set forth, for example, i one or more of Tables 1, 2, 3, 6 or 7) associated with ASD. In one embodiment, a set of oligonucleotides comprises from about 5 to about 30 oligonucleotides, from about 10 to about 20 oligonucleotides, and in one embodiment comprises about 20 oligonucleotides, all of which specifically hybridize to a particular genetic marker associated with ASD. Thus, a set of oligonucleotides may comprise about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200 or more oligonucleotides, all of which specifically hybridize to a particular SNP associated with ASD. In one embodiment, a set of oligonucleotides comprises DNA probes. In one embodiment, the DNA probes comprise overlapping DNA probes. In another embodiment, the DNA probes comprise nonoverlapping DNA probes. In one embodiment, the DNA probes provide detection coverage over the length of a SNP genetic marker associated with ASD. In another embodiment, a set of oligonucleotides comprises amplification primers that amplify a SNP genetic marker associated with ASD. In this regard, sets of oligonucleotides comprising amplification primers may comprise multiplex amplification primers. In another embodiment, the sets of oligonucleotides or DNA probes may be provided on an array, such as solid phase arrays, chromosomal/DNA microarrays, or micro-bead arrays. Array technology is well known in the art. Illustrative arrays contemplated for use in the present invention include, but are not limited to, arrays available from Affymetrix (Santa Clara, Calif.) and Illumina (San Diego, Calif.).

In one embodiment, hybridization on a microarray is used to detect the presence of one or more SNPs in a patient's sample. The term “microarray” refers to an ordered arrangement of hybridizable array elements, e.g., polynucleotide probes, on a substrate.

In another embodiment of the invention, constant denaturant capillary electrophoresis (CDCE) can be combined with high-fidelity PCR (HiFi-PCR) to detect the presence of one or more SNPs. In another embodiment, high-fidelity PCR is used. In yet another embodiment, denaturing HPLC, denaturing capillary electrophoresis, cycling temperature capillary electrophoresis, allele-specific PCRs, quantitative real time PCR approaches such as TaqMan® is employed to detect a SNP. Other approaches to detect the presence of one or more SNPs amenable for use with the present invention include polony sequencing approaches, microarray approaches, mass spectrometry, high-throughput sequencing approaches, e.g., at a single molecule level, are used.

In one embodiment, a reagent for detecting the one or more SNPs, e.g., two or more, three or more or four or more SNPs, comprises one or more oligonucleotides, wherein each oligonucleotide specifically hybridizes to a SNP genetic marker associated with ASD. As will be understood by one of ordinary skill in the art, the one or more oligonucleotides is designed to hybridize to a gene at a position

Hybridization detection methods are based on the formation of specific hybrids between complementary nucleic acid sequences that serve to detect nucleic acid sequence mutation(s). Methods of nucleic acid analysis to detect polymorphisms and/or polymorphic variants include, e.g., microarray analysis and real time PCR. Hybridization methods, such as Southern analysis, Northern analysis, or in situ hybridizations, can also be used (see Current Protocols in Molecular Biology, Ausubel et al., eds., John Wiley & Sons 2003, incorporated by reference in its entirety).

Other methods include direct manual sequencing (Church and Gilbert, Proc. Natl. Acad. Sci. USA 81:1991-1995 (1988); Sanger et al., Proc. Natl. Acad. Sci. USA 74:5463-5467 (1977); Beavis et al. U.S. Pat. No. 5,288,644, each incorporated by reference in its entirety for all purposes); automated fluorescent sequencing; single-stranded conformation polymorphism assays (SSCP); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., Proc. Natl. Acad. Sci. USA 86:232-236 (1989)), mobility shift analysis (Orita et al., Proc. Natl. Acad. Sci. USA 86:2766-2770 (1989), incorporated by reference in its entirety), restriction enzyme analysis (Flavell et al., Cell 15:25 (1978); Geever et al., Proc. Natl. Acad. Sci. USA 78:5081 (1981), incorporated by reference in its entirety); quantitative real-time PCR (Raca et al., Genet Test 8(4):387-94 (2004), incorporated by reference in its entirety); heteroduplex analysis; chemical mismatch cleavage (CMC) (Cotton et al., Proc. Natl. Acad. Sci. USA 85:4397-4401 (1985), incorporated by reference in its entirety); RNase protection assays (Myers et al., Science 230:1242 (1985), incorporated by reference in its entirety); use of polypeptides that recognize nucleotide mismatches, e.g., E. coli mutS protein; allele-specific PCR, for example. See, e.g., U.S. Patent Publication No. 2004/0014095, which is incorporated herein by reference in its entirety.

In order to detect polymorphisms and/or polymorphic variants, in one embodiment, genomic DNA (gDNA) or a portion thereof containing the polymorphic site, present in the sample obtained from the subject, is first amplified. The polymorphic variant, in one embodiment, is one or more of the SNPs set forth in one of Tables 1, 2, 3, 6 or 7. Such regions can be amplified and isolated by PCR using oligonucleotide primers designed based on genomic and/or cDNA sequences that flank the site. See e.g., PCR Primer: A Laboratory Manual, Dieffenbach and Dveksler, (Eds.); McPherson et al., PCR Basics: From Background to Bench (Springer Verlag, 2000, incorporated by reference in its entirety); Mattila et al., Nucleic Acids Res., 19:4967 (1991), incorporated by reference in its entirety; Eckert et al., PCR Methods and Applications, 1:17 (1991), incorporated by reference in its entirety; PCR (eds. McPherson et al., IRL Press, Oxford), incorporated by reference in its entirety; and U.S. Pat. No. 4,683,202, incorporated by reference in its entirety. Other amplification methods that may be employed include the ligase chain reaction (LCR) (Wu and Wallace, Genomics, 4:560 (1989), Landegren et al., Science, 241:1077 (1988), transcription amplification (Kwoh et al., Proc. Natl. Acad. Sci. USA, 86:1173 (1989)), self-sustained sequence replication (Guatelli et al., Proc. Nat. Acad. Sci. USA, 87:1874 (1990)), incorporated by reference in its entirety, and nucleic acid based sequence amplification (NASBA). Guidelines for selecting primers for PCR amplification are known to those of ordinary skill in the art. See, e.g., McPherson et al., PCR Basics: From Background to Bench, Springer-Verlag, 2000, incorporated by reference in its entirety. A variety of computer programs for designing primers are available.

In one example, a sample (e.g., a sample comprising genomic DNA), is obtained from a subject. The DNA in the sample is then examined to determine SNP profile and optionally a CNV profile as described herein. The profile is determined by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular polymorphic variant.

In some embodiments, restriction digest analysis can be used to detect the existence of a polymorphic variant of a polymorphism, if alternate polymorphic variants of the polymorphism result in the creation or elimination of a restriction site. A sample containing genomic DNA is obtained from the individual. Polymerase chain reaction (PCR) can be used to amplify a region comprising the polymorphic site, and restriction fragment length polymorphism analysis is conducted (see Current Protocols in Molecular Biology, Ausubel et al., eds., John Wiley & Sons 2003, incorporated by reference in its entirety). The digestion pattern of the relevant DNA fragment indicates the presence or absence of a particular polymorphic variant of the polymorphism and is therefore indicative of the presence or absence of susceptibility to SZ.

Sequence analysis can also be used to detect the one or more SNPs, e.g., the one or more SNPs set forth in Tables 1, 2, 3, 6 or 7. A sample comprising DNA or RNA is obtained from the subject. PCR or other appropriate methods can be used to amplify a portion encompassing the polymorphic site, if desired. The sequence is then ascertained, using any standard method, and the presence of a polymorphic variant is determined.

Allele-specific oligonucleotides can also be used to detect the presence of a polymorphic variant, e.g., through the use of dot-blot hybridization of amplified oligonucleotides with allele-specific oligonucleotide (ASO) probes (see, for example, Saiki et al., Nature (London) 324:163-166 (1986)). An “allele-specific oligonucleotide” (also referred to herein as an “allele-specific oligonucleotide probe”) is typically an oligonucleotide of approximately 10-50 base pairs, preferably approximately 15-30 base pairs, that specifically hybridizes to a nucleic acid region that contains a polymorphism. An allele-specific oligonucleotide probe that is specific for particular a polymorphism can be prepared using standard methods (see Current Protocols in Molecular Biology, Ausubel et al., eds., John Wiley & Sons 2003, incorporated by reference in its entirety).

Generally, to determine which of multiple SNP variants is present in a subject, a sample comprising DNA is obtained from the subject. PCR or another amplification procedure can be used to amplify a portion encompassing the polymorphic site.

Real-time pyrophosphate DNA sequencing is yet another approach to detection of polymorphisms and polymorphic variants (Alderborn et al., (2000) Genome Research, 10(8):1249-1258, incorporated by reference in its entirety). Additional methods include, for example, PCR amplification in combination with denaturing high performance liquid chromatography (dHPLC) (Underhill et al., Genome Research, Vol. 7, No. 10, pp. 996-1005, 1997, incorporated by reference in its entirety for all purposes).

High throughput sequencing, or next-generation sequencing can also be employed to detect one or more of the SNPs described herein. Such methods are known in the art (see e.g., Zhang et al., J Genet Genomics. 2011 Mar. 20;38(3):95-109, incorporated by reference in its entirety for all purposes; Metzker, Nat Rev Genet. 2010 January; 11(1):31-46, incorporated by reference in its entirety for all purposes) and include, but are not limited to, technologies such as ABI SOLiD sequencing technology (now owned by Life Technologies, Carlsbad, Calif.); Roche 454 FLX which uses sequencing by synthesis technology known as pyrosequencing (Roche, Basel Switzerland); Illumina Genome Analyzer (Illumina, San Diego, Calif.); Dover Systems Polonator G.007 (Salem, N.H.); Helicos (Helicos BioSciences Corporation, Cambridge Mass., USA), and Sanger. In one embodiment, DNA sequencing may be performed using methods well known in the art including mass spectrometry technology and whole genome sequencing technologies, single molecule sequencing, etc.

In one embodiment, nucleic acid, for example, genomic DNA is sequenced using nanopore sequencing, to determine the presence of the one or more SNPs, and in some instances, the one or more CNVs (e.g., as described in Soni et al. (2007). Clin Chem 53, pp. 1996-2001, incorporated by reference in its entirety for all purposes). Nanopore sequencing is a single-molecule sequencing technology whereby a single molecule of DNA is sequenced directly as it passes through a nanopore. A nanopore is a small hole, of the order of 1 nanometer in diameter. Immersion of a nanopore in a conducting fluid and application of a potential (voltage) across it results in a slight electrical current due to conduction of ions through the nanopore. The amount of current which flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence. Nanopore sequencing technology as disclosed in U.S. Pat. Nos. 5,795,782, 6,015,714, 6,627,067, 7,238,485 and 7,258,838 and U.S. patent application publications U.S. Patent Application Publication Nos. 2006/003171 and 2009/0029477, each incorporated by reference in its entirety for all purposes, is amenable for use with the methods described herein.

Nucleic acid probes can be used to detect and/or quantify the presence of a particular target nucleic acid sequence within a sample of nucleic acid sequences, e.g., as hybridization probes, or to amplify a particular target sequence within a sample, e.g., as a primer. Probes have a complimentary nucleic acid sequence that selectively hybridizes to the target nucleic acid sequence. In order for a probe to hybridize to a target sequence, the hybridization probe must have sufficient identity with the target sequence, i.e., at least 70%, e.g., 80%, 90%, 95%, 98% or more identity to the target sequence. The probe sequence must also be sufficiently long so that the probe exhibits selectivity for the target sequence over non-target sequences. For example, the probe will be at least 10, e.g., 15, 20, 25, 30, 35, 50, 100, or more, nucleotides in length. In some embodiments, the probes are not more than 30, 50, 100, 200, 300, or 500 nucleotides in length. Probes include primers, which generally refers to a single-stranded oligonucleotide probe that can act as a point of initiation of template-directed DNA synthesis using methods such as PCR (polymerase chain reaction), LCR (ligase chain reaction), etc., for amplification of a target sequence.

In some embodiments, the probe is a test probe, e.g., a probe that can be used to detect polymorphisms in a region described herein, e.g., polymorphisms as described herein, for example, one or more, two or more, five or more, ten or more or twenty or more of the SNPs set forth in one of Tables 1, 2, 3, 6 or 7. In some embodiments, the probe can hybridize to a target sequence within a region delimited by delimiting SNPs, SNP1 and SNP2, inclusive as specified for the particular genes in Table 1 or SNPs of Tables 1, 2, 3, 6 or 7.

Control probes can also be used. For example, a probe that binds a less variable sequence, e.g., repetitive DNA associated with a centromere of a chromosome, or a probe that exhibits differential binding to the polymorphic site being interrogated, can be used as a control. Probes that hybridize with various centromeric DNA and locus-specific DNA are available commercially, for example, from Vysis, Inc. (Downers Grove, Ill.), Molecular Probes, Inc. (Eugene, Oreg.), or from Cytocell (Oxfordshire, UK).

In some embodiments, the probes are labeled with a “detectable label,” e.g., by direct labeling. In various embodiments, the oligonucleotides for detecting the one or more SNP genetic markers associated with ASD described herein are conjugated to a detectable label that may be detected directly or indirectly. In the present invention, oligonucleotides may all be covalently linked to a detectable label.

A “detectable label” is a molecule or material that can produce a detectable (such as visually, electronically or otherwise) signal that indicates the presence and/or concentration of the label in a sample. When conjugated to a nucleic acid such as a DNA probe, the detectable label can be used to locate and/or quantify a target nucleic acid sequence to which the specific probe is directed. Thereby, the presence and/or amount of the target in a sample can be detected by detecting the signal produced by the detectable label. A detectable label can be detected directly or indirectly, and several different detectable labels conjugated to different probes can be used in combination to detect one or more targets.

One type of “detectable label” is a fluorophore, an organic molecule that fluoresces after absorbing light of lower wavelength/higher energy. A directly labeled fluorophore allows the probe to be visualized without a secondary detection molecule. After covalently attaching a fluorophore to a nucleotide, the nucleotide can be directly incorporated into the probe with standard techniques such as nick translation, random priming, and PCR labeling. Alternatively, deoxycytidine nucleotides within the probe can be transaminated with a linker. The fluorophore then is covalently attached to the transaminated deoxycytidine nucleotides. See, e.g., U.S. Pat. No. 5,491,224, incorporated by reference in its entirety.

Examples of fluorescent labels include 5-(and 6)-carboxyfluorescein, 5- or 6-carboxyfluorescein, 6-(fluorescein)-5-(and 6)-carboxamido hexanoic acid, fluorescein isothiocyanate, rhodamine, tetramethylrhodamine, and dyes such as Cy2, Cy3, and Cy5, optionally substituted coumarin including AMCA, PerCP, phycobiliproteins including R-phycoerythrin (RPE) and allophycoerythrin (APC), Texas Red, Princeton Red, green fluorescent protein (GFP) and analogues thereof, and conjugates of R-phycoerythrin or allophycoerythrin, inorganic fluorescent labels such as particles based on semiconductor material like coated CdSe nanocrystallites.

Other examples of detectable labels, which may be detected directly, include radioactive substances and metal particles. In contrast, indirect detection requires the application of one or more additional probes or antibodies, i.e., secondary antibodies, after application of the primary probe or antibody. Thus, in certain embodiments, as would be understood by the skilled artisan, the detection is performed by the detection of the binding of the secondary probe or binding agent to the primary detectable probe. Examples of primary detectable binding agents or probes requiring addition of a secondary binding agent or antibody include enzymatic detectable binding agents and hapten detectable binding agents or antibodies.

In some embodiments, the detectable label is conjugated to a nucleic acid polymer which comprises the first binding agent (e.g., in an ISH, WISH, or FISH process). In other embodiments, the detectable label is conjugated to an antibody which comprises the first binding agent (e.g., in an IHC process).

Examples of detectable labels which may be conjugated to the oligonucleotides used in the methods of the present disclosure include fluorescent labels, enzyme labels, radioisotopes, chemiluminescent labels, electrochemiluminescent labels, bioluminescent labels, polymers, polymer particles, metal particles, haptens, and dyes.

Examples of polymer particle labels include micro particles or latex particles of polystyrene, PMMA or silica, which can be embedded with fluorescent dyes, or polymer micelles or capsules which contain dyes, enzymes or substrates.

Examples of metal particle labels include gold particles and coated gold particles, which can be converted by silver stains. Examples of haptens include DNP, fluorescein isothiocyanate (FITC), biotin, and digoxigenin. Examples of enzymatic labels include horseradish peroxidase (HRP), alkaline phosphatase (ALP or AP), β-galactosidase (GAL), glucose-6-phosphate dehydrogenase, β-N-acetylglucosamimidase, β-glucuronidase, invertase, Xanthine Oxidase, firefly luciferase and glucose oxidase (GO). Examples of commonly used substrates for horseradishperoxidase include 3,3′-diaminobenzidine (DAB), diaminobenzidine with nickel enhancement, 3-amino-9-ethylcarbazole (AEC), Benzidine dihydrochloride (BDHC), Hanker-Yates reagent (HYR), Indophane blue (IB), tetramethylbenzidine (TMB), 4-chloro-1-naphtol (CN), α-naphtol pyronin (α-NP), o-dianisidine (OD), 5-bromo-4-chloro-3-indolylphosphate (BCIP), Nitro blue tetrazolium (NBT), 2-(p-iodophenyl)-3-p-nitropheny-1-5-phenyl tetrazolium chloride (INT), tetranitro blue tetrazolium (TNBT), 5-bromo-4-chloro-3-indoxyl-beta-D-galactoside/ferro-ferricyanide (BCIG/FF).

Examples of commonly used substrates for Alkaline Phosphatase include Naphthol-AS-B 1-phosphate/fast red TR (NABP/FR), Naphthol-AS-MX-phosphate/fast red TR (NAMP/FR), Naphthol-AS-B1-phosphate/-fast red TR (NABP/FR), Naphthol-AS-MX-phosphate/fast red TR (NAMP/FR), Naphthol-AS-B1-phosphate/new fuschin (NABP/NF), bromochloroindolyl phosphate/nitroblue tetrazolium (BCIP/NBT), 5-Bromo-4-chloro-3-indolyl-b-d-galactopyranoside (BCIG).

Examples of luminescent labels include luminol, isoluminol, acridinium esters, 1,2-dioxetanes and pyridopyridazines. Examples of electrochemiluminescent labels include ruthenium derivatives. Examples of radioactive labels include radioactive isotopes of iodide, cobalt, selenium, tritium, carbon, sulfur and phosphorous.

Detectable labels may be linked to any molecule that specifically binds to a biological marker of interest, e.g., an antibody, a nucleic acid probe, or a polymer. Furthermore, one of ordinary skill in the art would appreciate that detectable labels can also be conjugated to second, and/or third, and/or fourth, and/or fifth binding agents, nucleic acids, or antibodies, etc. Moreover, the skilled artisan would appreciate that each additional binding agent or nucleic acid used to characterize a biological marker of interest (e.g., the one or more SNP genetic markers associated with ASD as set forth in one or more of Tables 1, 2, 3, 6 or 7) may serve as a signal amplification step. The biological marker may be detected visually using, e.g., light microscopy, fluorescent microscopy, electron microscopy where the detectable substance is for example a dye, a colloidal gold particle, a luminescent reagent. Visually detectable substances bound to a biological marker may also be detected using a spectrophotometer. Where the detectable substance is a radioactive isotope, detection can be visually by autoradiography, or non-visually using a scintillation counter. See, e.g., Larsson, 1988, Immunocytochemistry: Theory and Practice, (CRC Press, Boca Raton, Fla.); Methods in Molecular Biology, vol. 80 1998, John D. Pound (ed.) (Humana Press, Totowa, N.J.), each incorporated by reference in their entireties for all purposes.

In other embodiments, the probes can be indirectly labeled with, e.g., biotin or digoxygenin, or labeled with radioactive isotopes such as ³²P and ³H. For example, a probe indirectly labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Oligonucleotide probes that exhibit differential or selective binding to polymorphic sites may readily be designed by one of ordinary skill in the art. For example, an oligonucleotide that is perfectly complementary to a sequence that encompasses a polymorphic site (i.e., a sequence that includes the polymorphic site, within it or at one end) will generally hybridize preferentially to a nucleic acid comprising that sequence, as opposed to a nucleic acid comprising an alternate polymorphic variant.

In another aspect, the invention features arrays that include a substrate having a plurality of addressable areas, and methods of using them. At least one area of the plurality includes a nucleic acid probe that binds specifically to a sequence comprising a polymorphism listed in Table 1, 2, 3, 6 or 7, and can be used to detect the absence or presence of said polymorphism, e.g., one or more SNPs, as described herein. For example, the array can include one or more nucleic acid probes that can be used to detect a polymorphism listed in Table 1 or 2. In some embodiments, the array further includes at least one area that includes a nucleic acid probe that can be used to specifically detect another marker associated with ASD, for example, a copy number variant (CNV), for example one or more of the CNVs described in either U.S. Patent Application Publication No. 2010/0210471 and/or International PCT publication no. 2014/055915, each incorporated by reference in their entireties for all purposes. The substrate can be, e.g., a two-dimensional substrate known in the art such as a glass slide, a wafer (e.g., silica or plastic), a mass spectroscopy plate, or a three-dimensional substrate such as a gel pad. In some embodiments, the probes are nucleic acid capture probes.

Methods for generating arrays are known in the art and include, e.g., photolithographic methods (see, e.g., U.S. Pat. Nos. 5,143,854; 5,510,270; and 5,527,681, each of which is incorporated by reference in its entirety), mechanical methods (e.g., directed-flow methods as described in U.S. Pat. No. 5,384,261), pin-based methods (e.g., as described in U.S. Pat. No. 5,288,514, incorporated by reference in its entirety), and bead-based techniques (e.g., as described in PCT US/93/04145, incorporated by reference in its entirety). The array typically includes oligonucleotide probes capable of specifically hybridizing to different polymorphic variants. According to the method, a nucleic acid of interest, e.g., a nucleic acid encompassing a polymorphic site, (which is typically amplified) is hybridized with the array and scanned. Hybridization and scanning are generally carried out according to standard methods. After hybridization and washing, the array is scanned to determine the position on the array to which the nucleic acid from the sample hybridizes. The hybridization data obtained from the scan is typically in the form of fluorescence intensities as a function of location on the array.

Arrays can include multiple detection blocks (i.e., multiple groups of probes designed for detection of particular polymorphisms). Such arrays can be used to analyze multiple different polymorphisms, e.g., distinct polymorphisms at the same polymorphic site or polymorphisms at different chromosomal sites. Detection blocks may be grouped within a single array or in multiple, separate arrays so that varying conditions (e.g., conditions optimized for particular polymorphisms) may be used during the hybridization.

Additional description of use of oligonucleotide arrays for detection of polymorphisms can be found, for example, in U.S. Pat. Nos. 5,858,659 and 5,837,832, each of which is incorporated by reference in its entirety.

Results of the SNP and/or CNV profiling performed on a sample from a subject (test sample) may be compared to a biological sample(s) or data derived from a biological sample(s) that is known or suspected to be normal (“reference sample” or “normal sample”). In some embodiments, a reference sample is a sample that is not obtained from an individual having an ASD, or would test negative in the SNP profiling assay for the one or more SNPs under evaluation. The reference sample may be assayed at the same time, or at a different time from the test sample.

The results of an assay on the test sample may be compared to the results of the same assay on a reference sample. In some cases, the results of the assay on the reference sample are from a database, or a reference. In some cases, the results of the assay on the reference sample are a known or generally accepted value or range of values by those skilled in the art. In some cases the comparison is qualitative. In other cases the comparison is quantitative. In some cases, qualitative or quantitative comparisons may involve but are not limited to one or more of the following: comparing fluorescence values, spot intensities, absorbance values, chemiluminescent signals, histograms, critical threshold values, statistical significance values, SNP presence or absence, copy number variations.

In one embodiment, an odds ratio (OR) is calculated for each individual SNP measurement. Here, the OR is a measure of association between the presence or absence of an SNP, and an outcome, e.g., ASD positive or ASD negative. Odds ratios are most commonly used in case-control studies. For example, see, J. Can. Acad Child Adolesc. Psychiatry 2010; 19(3): 227-229, which is incorporated by reference in its entirety for all purposes. Odds ratios for each SNP can be combined to make an ultimate ASD diagnosis.

In one embodiment, a specified statistical confidence level may be determined in order to provide a diagnostic confidence level. For example, it may be determined that a confidence level of greater than 90% may be a useful predictor of the presence of ASD or the likelihood that a subject will develop ASD. In other embodiments, more or less stringent confidence levels may be chosen. For example, a confidence level of about or at least about 50%, 60%, 70%, 75%, 80%, 85%, 90%, 95%, 97.5%, 99%, 99.5%, or 99.9% may be chosen as a useful phenotypic predictor. The confidence level provided may in some cases be related to the quality of the sample, the quality of the data, the quality of the analysis, the specific methods used, and/or the number of SNPs and optionally CNVs, analyzed. The specified confidence level for providing a diagnosis may be chosen on the basis of the expected number of false positives or false negatives and/or cost. Methods for choosing parameters for achieving a specified confidence level or for identifying markers with diagnostic power include but are not limited to Receiver Operating Characteristic (ROC) curve analysis, binormal ROC, principal component analysis, odds ratio analysis, partial least squares analysis, singular value decomposition, least absolute shrinkage and selection operator analysis, least angle regression, and the threshold gradient directed regularization method.

SNP and CNV detection may in some cases be improved through the application of algorithms designed to normalize and or improve the reliability of the data. In some embodiments of the present disclosure the data analysis requires a computer or other device, machine or apparatus for application of the various algorithms described herein due to the large number of individual data points that are processed. A “machine learning algorithm” refers to a computational-based prediction methodology, also known to persons skilled in the art as a “classifier,” employed for characterizing an SNP or SNP/CNV profile. The signals corresponding to certain SNPs or SNPs/CNVs, which are obtained by, e.g., microarray-based hybridization assays, are in one embodiment subjected to the algorithm in order to classify the profile. Supervised learning generally involves “training” a classifier to recognize the distinctions among classes (e.g., ASD positive, ASD negative, particular ASD subtype) and then “testing” the accuracy of the classifier on an independent test set. For new, unknown samples the classifier can be used to predict the class (e.g., ASD positive, ASD negative, particular ASD subtype) in which the samples belong.

In some embodiments, a robust multi-array average (RMA) method may be used to normalize raw data. The RMA method begins by computing background-corrected intensities for each matched cell on a number of microarrays. In one embodiment, the background corrected values are restricted to positive values as described by Irizarry et al. (2003). Biostatistics April 4 (2): 249-64, incorporated by reference in its entirety for all purposes. After background correction, the base-2 logarithm of each background corrected matched-cell intensity is then obtained. The background corrected, log-transformed, matched intensity on each microarray is then normalized using the quantile normalization method in which for each input array and each probe value, the array percentile probe value is replaced with the average of all array percentile points, this method is more completely described by Bolstad et al. Bioinformatics 2003, incorporated by reference in its entirety. Following quantile normalization, the normalized data may then be fit to a linear model to obtain an intensity measure for each probe on each microarray. Tukey's median polish algorithm (Tukey, J. W., Exploratory Data Analysis. 1977, incorporated by reference in its entirety) may then be used to determine the log-scale intensity level for the normalized probe set data.

Various other software programs may be implemented. In certain methods, feature selection and model estimation may be performed by logistic regression with lasso penalty using glmnet (Friedman et al. (2010). Journal of statistical software 33(1): 1-22, incorporated by reference in its entirety). Raw reads may be aligned using TopHat (Trapnell et al. (2009). Bioinformatics 25(9): 1105-11, incorporated by reference in its entirety). In methods, top features (N ranging from 10 to 200) are used to train a linear support vector machine (SVM) (Suykens J A K, Vandewalle J. Least Squares Support Vector Machine Classifiers. Neural Processing Letters 1999; 9(3): 293-300, incorporated by reference in its entirety) using the e1071 library (Meyer D. Support vector machines: the interface to libsvm in package e1071. 2014, incorporated by reference in its entirety). Confidence intervals may be computed using the pROC package (Robin X, Turck N, Hainard A, et al. pROC: an open-source package for R and S+ to analyze and compare ROC curves. BMC bioinformatics 2011; 12: 77, incorporated by reference in its entirety).

In addition, data may be filtered to remove data that may be considered suspect. In some embodiments, data deriving from microarray probes that have fewer than about 4, 5, 6, 7 or 8 guanosine+cytosine nucleotides may be considered to be unreliable due to their aberrant hybridization propensity or secondary structure issues. Similarly, data deriving from microarray probes that have more than about 12, 13, 14, 15, 16, 17, 18, 19, 20, 21, or 22 guanosine+cytosine nucleotides may be considered unreliable due to their aberrant hybridization propensity or secondary structure issues.

In some embodiments of the present invention, data from probe-sets may be excluded from analysis if they are not identified at a detectable level (above background).

In some embodiments of the present disclosure, probe-sets that exhibit no, or low variance may be excluded from further analysis. Low-variance probe-sets are excluded from the analysis via a Chi-Square test. In one embodiment, a probe-set is considered to be low-variance if its transformed variance is to the left of the 99 percent confidence interval of the Chi-Squared distribution with (N−1) degrees of freedom. (N−1)*Probe-set Variance/(Gene Probe-set Variance). about.Chi-Sq(N−1) where N is the number of input CEL files, (N−1) is the degrees of freedom for the Chi-Squared distribution, and the “probe-set variance for the gene” is the average of probe-set variances across the gene. In some embodiments of the present invention, probe-sets for a given SNP or group of SNPs may be excluded from further analysis if they contain less than a minimum number of probes that pass through the previously described filter steps for GC content, reliability, variance and the like. For example in some embodiments, probe-sets for a given gene or transcript cluster may be excluded from further analysis if they contain less than about 1, 2, 3, 4, 5, 6, 7, 8, 9, 10, 11, 12, 13, 14, 15, or less than about 20 probes.

Methods of SNP and optionally CNV data analysis may further include the use of a feature selection algorithm as provided herein. In some embodiments of the present invention, feature selection is provided by use of the LIMMA software package (Smyth, G. K. (2005). Limma: linear models for microarray data. In: Bioinformatics and Computational Biology Solutions using R and Bioconductor, R. Gentleman, V. Carey, S. Dudoit, R. Irizarry, W. Huber (eds.), Springer, New York, pages 397-420, incorporated by reference in its entirety for all purposes).

Methods of SNP and optionally CNV data analysis of may further include the use of a pre-classifier algorithm. For example, an algorithm may use a specific molecular fingerprint to pre-classify the samples according to their composition and then apply a correction/normalization factor. This data/information may then be fed in to a final classification algorithm which would incorporate that information to aid in the final diagnosis.

Methods of SNP and optionally CNV data analysis may further include the use of a classifier algorithm as provided herein. In some embodiments of the present invention a diagonal linear discriminant analysis, k-nearest neighbor algorithm, support vector machine (SVM) algorithm, linear support vector machine, random forest algorithm, or a probabilistic model-based method or a combination thereof is provided for classification of microarray data. In some embodiments, identified markers that distinguish samples (e.g., ASD positive from normal) are selected based on statistical significance of the difference in expression levels between classes of interest. In some cases, the statistical significance is adjusted by applying a Benjamin Hochberg or another correction for false discovery rate (FDR).

In some cases, the classifier algorithm may be supplemented with a meta-analysis approach such as that described by Fishel and Kaufman et al. 2007 Bioinformatics 23(13): 1599-606, incorporated by reference in its entirety for all purposes. In some cases, the classifier algorithm may be supplemented with a meta-analysis approach such as a repeatability analysis.

Methods for deriving and applying posterior probabilities to the analysis of microarray data are known in the art and have been described for example in Smyth, G. K. 2004 Stat. Appi. Genet. Mol. Biol. 3: Article 3, incorporated by reference in its entirety for all purposes. In some cases, the posterior probabilities may be used in the methods of the present invention to rank the markers provided by the classifier algorithm.

A statistical evaluation of the results of the molecular profiling may provide a quantitative value or values indicative of one or more of the following: the likelihood of diagnostic accuracy of ASD; the likelihood of a particular ASD (e.g., autistic disorders vs. AS); the likelihood of the success of a particular therapeutic intervention. In one embodiment, the data is presented directly to the physician in its most useful form to guide patient care. The results of the molecular profiling can be statistically evaluated using a number of methods known to the art including, but not limited to: the students T test, the two sided T test, pearson rank sum analysis, hidden markov model analysis, analysis of q-q plots, principal component analysis, one way ANOVA, two way ANOVA, LIMMA and the like.

In some cases, accuracy may be determined by tracking the subject over time to determine the accuracy of the original diagnosis. In other cases, accuracy may be established in a deterministic manner or using statistical methods. For example, receiver operator characteristic (ROC) analysis may be used to determine the optimal assay parameters to achieve a specific level of accuracy, specificity, positive predictive value, negative predictive value, and/or false discovery rate.

In some cases the results of the SNP assays, are entered into a database for access by representatives or agents of a molecular profiling business, the individual, a medical provider, or insurance provider. In some cases assay results include sample classification, identification, or diagnosis by a representative, agent or consultant of the business, such as a medical professional. In other cases, a computer or algorithmic analysis of the data is provided automatically. In some cases the molecular profiling business may bill the individual, insurance provider, medical provider, researcher, or government entity for one or more of the following: molecular profiling assays performed, consulting services, data analysis, reporting of results, or database access.

In some embodiments of the present invention, the results of the SNP profiling are presented as a report on a computer screen or as a paper record. In some embodiments, the report may include, but is not limited to, such information as one or more of the following: the number of SNPs identified as compared to the reference sample, the suitability of the original sample, a diagnosis, a statistical confidence for the diagnosis, the likelihood of a particular ASD, and proposed therapies.

The results of the SNP profiling may be classified into one of the following: ASD positive, a particular type of ASD, a non-ASD sample, or non-diagnostic (providing inadequate information concerning the presence or absence of ASD).

In some embodiments of the present invention, results are classified using a trained algorithm. Trained algorithms of the present invention include algorithms that have been developed using a reference set of known ASD and normal samples, for example, samples from individuals diagnosed with a particular ASD subtype, ASD, or not diagnosed with ASD (ASD-negative). In some embodiments, training comprises comparison of SNPs in from a first ASD positive sample to SNPs in a second ASD positive sample, where the first set of SNPs includes at least one SNP that is not in the second set, and the SNPs are selected from the SNPs provided in Table 1, 2, 3, 6 or 7.

Algorithms suitable for categorization of samples include but are not limited to k-nearest neighbor algorithms, support vector machines, linear discriminant analysis, diagonal linear discriminant analysis, updown, naive Bayesian algorithms, neural network algorithms, hidden Markov model algorithms, genetic algorithms, or any combination thereof.

When classifying a biological sample for diagnosis of ASD, there are typically two possible outcomes from a binary classifier. When a binary classifier is compared with actual true values (e.g., values from a biological sample), there are typically four possible outcomes. If the outcome from a prediction is p (where “p” is a positive classifier output, such as the presence of ASD or a particular ASD) and the actual value is also p, then it is called a true positive (TP); however if the actual value is n then it is said to be a false positive (FP). Conversely, a true negative has occurred when both the prediction outcome and the actual value are n (where “n” is a negative classifier output, such as no ASD), and false negative is when the prediction outcome is n while the actual value is p. In one embodiment, consider a diagnostic test that seeks to determine whether a person has a certain ASD. A false positive in this case occurs when the person tests positive, but actually does not have the ASD. A false negative, on the other hand, occurs when the person tests negative, suggesting they are healthy, when they actually do have the disease (the ASD).

The positive predictive value (PPV), or precision rate, or post-test probability of disease, is the proportion of subjects with positive test results who are correctly diagnosed. It reflects the probability that a positive test reflects the underlying condition being tested for. Its value does however depend on the prevalence of the disease, which may vary. In one example the following characteristics are provided: FP (false positive); TN (true negative); TP (true positive); FN (false negative). False positive rate (α)=FP/(FP+TN)−specificity; False negative rate (β)=FN/(TP+FN)−sensitivity; Power=sensitivity=1−β; Likelihood-ratio positive=sensitivity/(1−specificity); Likelihood-ratio negative=(1−sensitivity)/specificity. The negative predictive value (NPV) is the proportion of subjects with negative test results who are correctly diagnosed.

In some embodiments, the results of the SNP analysis of the subject methods provide a statistical confidence level that a given diagnosis is correct. In some embodiments, such statistical confidence level is at least about, or more than about 85%, 90%, 91%, 92%, 93%, 94%, 95%, 96%, 97%, 98%, 99% 99.5%, or more.

In one embodiment, depending on the results of the SNP hybridization assay and data analysis, the subject is selected for treatment for a particular ASD.

In one embodiment, the subject is selected for the treatment of classic autism. Treatments include, e.g., gene therapy, RNA interference (RNAi), behavioral therapy (e.g., Applied Behavior Analysis (ABA), Discrete Trial Training (DTT), Early Intensive Behavioral Intervention (EMI), Pivotal Response Training (PRT), Verbal Behavior Intervention (VBI), and Developmental Individual Differences Relationship-Based Approach (DIR)), physical therapy, occupational therapy, sensory integration therapy, speech therapy, the Picture Exchange Communication System (PECS), dietary treatment, and drugs (e.g., antipsychotics, anti-depressants, anticonvulsants, stimulants).

In another embodiment, the subject is selected for the treatment of Asperger's disorder. Treatments include, e.g., gene therapy, RNAi, occupational therapy, physical therapy, communication and social skills training, cognitive behavioral therapy, speech or language therapy, and drugs (e.g., aripiprazole, guanfacine, selective serotonin reuptake inhibitors (SSRIs), riseridone, olanzapine, naltrexone).

In one embodiment, the subject is selected for the treatment of Rett's disorder. Treatments include, e.g., gene therapy, RNAi, occupational therapy, physical therapy, speech or language therapy, nutritional supplements, and drugs (e.g., SSRIs, anti-psychotics, beta-blockers, anticonvulsants).

In one embodiment, the subject is selected for the treatment of CDD. Treatments include, e.g., gene therapy, RNAi, behavioral therapy (e.g., ABA, DTT, EIBI, PRT, VBI, and DIR), sensory enrichment therapy, occupational therapy, physical therapy, speech or language therapy, nutritional supplements, and drugs (e.g., anti-psychotics and anticonvulsants).

In another embodiment, the subject is selected for the treatment of PDD-NOS. Treatments include, e.g., gene therapy, RNAi, behavioral therapy (e.g., ABA, DTT, EIBI, PRT, VBI, and DIR), physical therapy, occupational therapy, sensory integration therapy, speech therapy, PECS, dietary treatment, and drugs (e.g., antipsychotics, anti-depressants, anticonvulsants, stimulants)

In one embodiment, the treatment the subject is selected for is gene therapy to correct, replace, or compensate for a target gene, for example, a wild type allele of one of the genes in Table 1.

In one aspect, the present invention provides a diagnostic test. In one embodiment, the diagnostic test comprises one or more oligonucleotides for use in a hybridization assay. The one or more oligonucleotides are designed to hybridize to one or more of the SNPs (e.g., two or more, five or more, ten or more, fifteen or more or twenty or more) set forth in Table 1, 2, 3, 6 or 7. In a further embodiment, the one or more oligonucleotides (e.g., two or more, five or more, ten or more, fifteen or more or twenty or more) is present on a microarray. In one embodiment, the diagnostic test comprises one or more devices, tools, and equipment configured to collect a genetic sample from an individual. In one embodiment of a diagnostic test, tools to collect a genetic sample may include one or more of a swab, a scalpel, a syringe, a scraper, a container, and other devices and reagents designed to facilitate the collection, storage, and transport of a genetic sample. In one embodiment, a diagnostic test may include reagents or solutions for collecting, stabilizing, storing, and processing a genetic sample. Such reagents and solutions for collecting, stabilizing, storing, and processing genetic material are well known by those of skill in the art. In another embodiment, a diagnostic test as disclosed herein, may comprise a microarray apparatus and associated reagents, a flow cell apparatus and associated reagents, a multiplex next generation nucleic acid sequencer and associated reagents, and additional hardware and software necessary to assay a genetic sample for the presence of certain genetic markers and to detect and visualize certain genetic markers.

### Examples

The present invention is further illustrated by reference to the following Example. However, it should be noted that these Examples, like the embodiments described above, are illustrative and are not to be construed as restricting the scope of the invention in any way. The references cited in the Example are incorporated by reference in their entireties for all purposes.

In addition to single nucleotide variants and small insertions/deletions that can be identified by DNA sequencing, larger deletions or duplications (copy number variants, CNVs) have been shown to play a role in the etiology of ASDs [15-27]. Despite the observed inheritance of many ASD predisposition CNVs from an unaffected parent, the lack of extended, multi-generation pedigrees has precluded a comprehensive analysis of segregation of ASD predisposition CNVs and SNPs and the characterization of other genetic factors necessary for their expression. The large families available in Utah coupled with the willingness of family members to participate in genetic studies have resulted in the identification of a large number of disease predisposition genes for both Mendelian and complex diseases.

The pedigrees used in this study were part of a 70-family linkage study published previously [28] and two smaller studies that evaluated a single extended pedigree in this collection of families [29,30]. In this example, members of 26 extended multigenerational ASD families and four two-generation multiplex ASD families were analyzed by performing haplotype sharing analysis to identify chromosomal regions that potentially harbor ASD predisposition genes. DNA capture and sequencing of all genes in shared regions and of additional autism risk genes was then employed to identify SNPs that might predispose to ASD in these families. These SNPs were analyzed in a large case/control study and for segregation in these families. Also evaluated was the segregation of CNVs reported previously [27] in these families.

### Methods

### DNA Samples

A total of 386 DNA samples from 26 extended multi-generation and four 2-generation Utah multiplex ASD pedigrees were used in this study. Families were ascertained and recruited using the Utah Population Database (UPDB) as previously described [28]. Affection status was determined using the Autism Diagnostic Interview-Revised (ADI-R) and the Autism Diagnostic Observation Schedule (ADOS), for both the familial ASD cases and the unrelated ASD cases, as described previously [27]. The average number of affected individuals in each pedigree is 7.9. The pedigrees described here are a subset of those described previously [28]. Pedigree details are shown in Table 9.

A total of 9,000 DNA samples previously described in a case/control study [27], including 3,000 individuals with ASD and 6,000 controls, were used to evaluate these variants in a broader population. All samples collected for the work described here were collected under methods approved by the University of Utah Institutional Review Board (IRB) (University of Utah IRB #:6042-96) and the Children's Hospital of Philadelphia IRB (CHOP IRB #: IRB 06-004886). Patients and their families were recruited through the University of Utah Department of Psychiatry or the Children's Hospital of Philadelphia clinic or CHOP outreach clinics. Written informed consent was obtained from the participants or their parents using IRB approved consent forms prior to enrollment in the project. There was no discrimination against individuals or families who chose not to participate in the study. All data were analyzed anonymously and all clinical investigations were conducted according to the principles expressed in the Declaration of Helsinki.

### SNP Microarray Genotyping

Affymetrix 250K NspI SNP chip genotyping was carried out on all 386 DNA samples using the manufacturer's recommended procedure. Genotypes were called by Affymetrix Genotyping Console software using the BRLMM [31] genotype calling algorithm. Only SNPs with call rates greater than or equal to 99% were used for further analyses. SNPs demonstrating Mendelian errors also were identified using PedCheck [32] and were excluded.

### Shared Haplotype Analysis

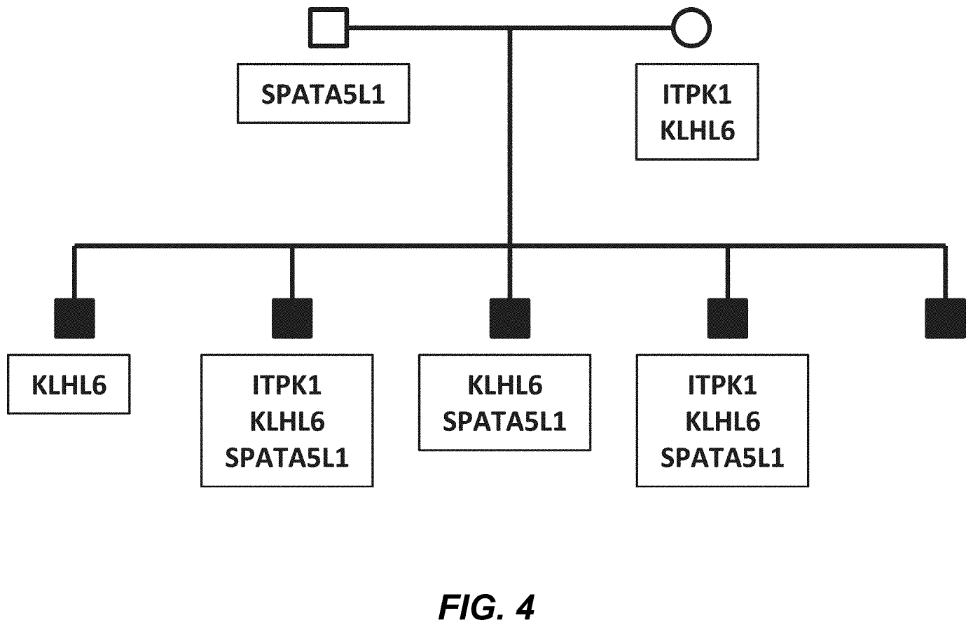

Shared haplotype analysis was performed on each pedigree, to identify genomic regions that have significant sharing among the affected individuals in that pedigree. The HapShare algorithm [33] was used to perform haplotype phasing based on Mendelian inheritance and to identify shared genomic segments. The comparisons included N out of N affected individuals, (N−1) out of N, (N−2) out of N, (N−3) out of N, and so on (See FIG. 4 in [33]). In 2-generation pedigrees, in some cases co-segregation of haplotypes was observed in all affected individuals analyzed, but the shared regions were large, including up to half of a chromosome. Consequently, shared regions from nuclear families were not selected for sequencing unless they overlapped regions observed in additional families.

### Custom Targeted Exome DNA Sequencing

NimbleGen custom sequence capture arrays were designed to capture 2,000 base pairs upstream of the transcription start site and all exons and exon-intron boundaries of genes within the shared genomic segments. An additional 23 genes from outside of the haplotype sharing regions were selected from the literature based on their potential roles in autism or neuronal functions (see Table 10). A total of approximately 1,800 genes were captured. Capture and Illumina DNA sequencing were performed by the Vanderbilt University Microarray Shared Resource facility on DNA from 26 affected individuals from 11 families that showed sharing of genomic segments. Short reads were aligned to the National Cancer Biotechnology Information (NCBI) reference human genome build 36 (GRCh36/hg18) and variants were called using the software alignment and variant calling methods described in Table 4 [34-36]. Potential variants detected by at least two of the methods were selected for further analysis.

### Variant Annotation

In silico functional analysis was carried out initially using cSNP classifier, a preliminary program later incorporated into VAAST [37], to classify variants as synonymous, conservative missense, non-conservative missense, nonsense, frameshift, or splice site mutations. Later, variants were re-annotated using the ANNOVAR program [38]. The KnownGene and RefSeq gene tracks from the UCSC genome browser were used to annotate functional variants, and the LiftOver tool was used to convert human genome build 36 (GRCh36/hg18) coordinates to human genome build 37 (GRCh37/hg19) coordinates [39,40].

### Custom Microarray Design and Array Processing

Design of the custom iSelect Infinium™ II BeadChip array (Illumina Inc.) including probes for 2,799 functional SNPs and 7,134 CNV probes was described previously [27]. The custom iSelect array was previously processed on 3,000 case and 6,000 control samples at the Center for Applied Genomics at Children's Hospital of Philadelphia (CHOP) [27].

The same array was also used to analyze DNA from 196 Utah discovery cohort family members at the University of Utah Genomics Core facility for variant validation and analysis of SNP segregation in families.

### Array Data Quality Control

Sample QC

Subjects were withheld from SNP analysis if any of the following were true: (1) subsequent to genotyping, the DNA sample was of apparent poor quality, evidenced by very low call rates (N=134); (2) the subject was identified as a trisomy-21 (N=51); (3) the subject was outside of the central cluster of Caucasian subjects identified by principal component analysis (PCA) (N=903) [27].

Relatedness estimation further indicated that some of the case subjects and controls were part of families with multiple relatives represented in the data. Re-evaluation of family structure in the sample cohorts used subsequently identified additional relationships. Subsequent association tests were therefore conducted using only one member of each known family in order to reduce the possibility of statistical confounding due to relatedness. For these tests, the subject selected from each family was the individual located nearest to the median centroid of the first two principal components. The number of subjects removed due to relatedness was 688. This resulted in a final sample set for association testing comprising 7326 subjects, of which 1541 were cases and 5785 were controls.

Principal component analysis (PCA) was used to avoid artifacts due to population stratification. Principal components were calculated in Golden Helix SNP and Variation Suite (SVS) using default settings. All subjects were included in the calculation except those that failed sample QC. Prior to calculating principal components, the SNPs were filtered according to the following criteria: autosomes only, call rate >0.95, minor allele frequency (MAF)>0.05, linkage disequilibrium R²<25% for all pairs of SNPs within a moving window of 50 SNPs. Two thousand eight SNPs, including those used for CNV analysis, were used for the principal component calculations. No genotype data were available for reference populations. However, a self-reported ethnicity variable was available for most subjects. A plot of the first two principal components shows a primary central cluster of subjects, with outlier groups extending along two axes. These roughly correspond to Asian and African-American ancestry as self-reported in the phenotype data. A simple outlier detection algorithm was applied to stratify the subjects into two groups representing the most probable Caucasians and non-Caucasians. This was done by first calculating the Cartesian distance of each subject from the median centroid of the first two principal component vectors. After determining the third quartile (Q3) and inter-quartile range (IQR) of the distances, any subject with a distance exceeding Q3+1.5×IQR was determined to be outside of the main cluster, and therefore non-Caucasian. Six hundred eighty-two subjects were placed in the non-Caucasian category. A graphical representation of the results of this PCA analysis were reported previously [27].

### SNP Quality Control (QC)

Prior to association testing, SNPs were evaluated for call rate, Hardy-Weinberg equilibrium (HWE) and allele frequency. All SNPs with call rates lower than 99% were removed from further analysis. No SNPs had significant Hardy-Weinberg disequilibrium.

### Laboratory Confirmation of SNPs and CNVs

For molecular validation of SNPs, PCR products were first screened by LightScanner High Resolution Melt curve analysis (BioFire Diagnostics Inc.) for the presence of sequence variants. PCR primer sequences are shown in Table 3. Any samples that gave abnormal melt profiles were sequenced using the Sanger method to confirm the presence of a sequence variant. For CNVs, pre- or custom-designed TaqMan copy number assays (Applies Biosystems Inc.) were used as described previously [27].

### Protein Binding Assay

All GST-tagged proteins were expressed and purified as described previously [41]. To test Rab11FIP5 binding to various Rab GTPases, purified recombinant FIP5(490-653) or FIP5(490-653)-P652L were incubated with glutathione beads coated with GST, GST-Rab11a, GST-Rab4a or GST-Rab3a in the presence of 1 μm GMP-PNP. Beads were then washed with phosphate-buffered saline and eluted with 1% SDS. Eluates were then analyzed for the presence of FIP5(490-653) by immunoblotting with anti-Rab11FIP5 antibodies. A similar assay also was used to test the ability of Rab11FIP5 (wild-type or P652L mutant) to dimerize.

### Flow Cytometry Analysis of Transferrin Recycling

To test the effect of the Rab11FIP5-P652L mutant on endocytic recycling, the transferrin recycling assay was used as described previously [42]. Briefly, HeLa cells expressing either wild-type FIP5-GFP or FIP5-GFP-P652L were incubated with transferrin conjugated to Alexa488. Cells were then washed and incubated with serum-supplemented media for varying amounts of time. The cell-associated (not recycled) Tf-Alexa488 was analyzed by flow cytometry.

### Results

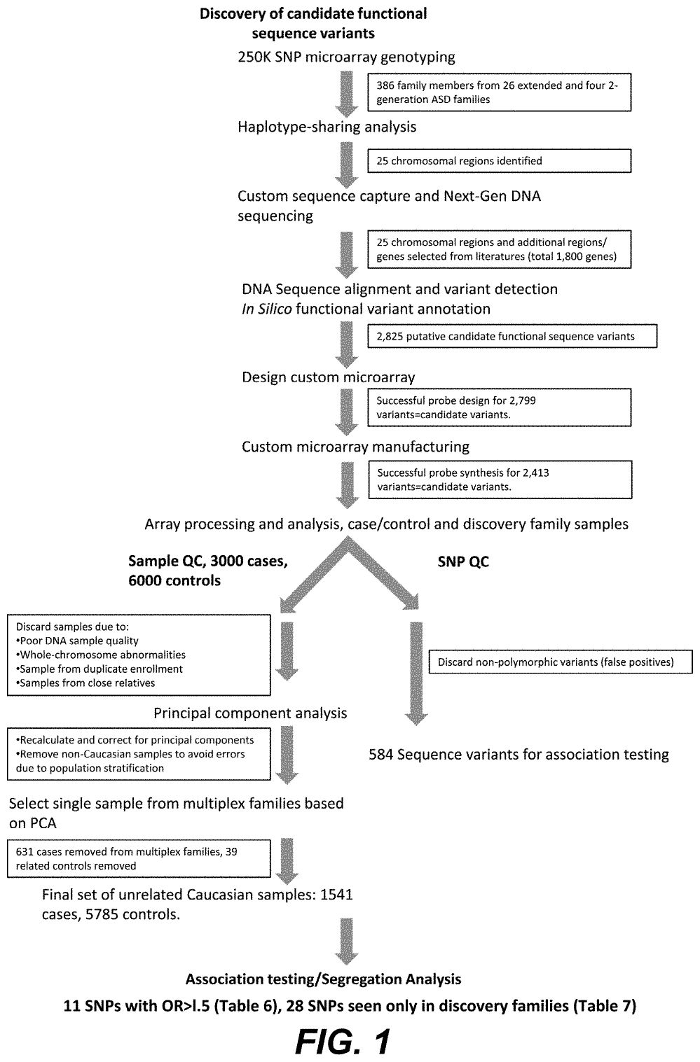

To identify genes that predispose to ASDs in multiplex ASD families, a haplotype sharing/custom DNA capture and sequencing approach was undertaken. The workflow outlined in FIG. 1 was undertaken, first to identify chromosomal regions with excessive sharing among affected individuals in multiplex ASD families. Sequence capture to identify potential functional sequence variants in the genes lying in the shared regions was then used, as well as to identify additional ASD genes. Finally, the segregation of those variants in ASD families was evaluated and their prevalence was determined in a large set of ASD cases and a large set of controls. The details of this process are described below.

### Affymetrix 250K SNP Genotyping and Haplotype Sharing

SNP genotyping was carried out on 386 DNA samples from 26 extended multi-generation and four 2-generation Utah multiplex ASD pedigrees. SNPs with no map location were not included in the analysis. The average call rate was 99.1% for the entire dataset.

The HapShare method [33] was used to identify genomic regions that have significant sharing among the affected individuals in each of the 30 pedigrees we studied. Paternal and maternal haplotypes were determined based on Mendelian inheritance using only informative markers. These haplotypes then were compared among affected individuals within each extended or nuclear family. Eighteen regions of haplotype sharing were selected based on sharing in extended pedigrees for further analysis. The degree of sharing that we observed among affected individuals and the coordinates of the regions selected for DNA capture and sequencing are shown in Table 5. Two additional regions were selected for DNA capture and sequencing based on a published linkage analysis using an overlapping set of families [28].

### Sequence Capture, Sequence Analysis and Variant Identification

Capture and DNA sequencing was performed using DNA from 26 affected individuals from 11 families that showed the best sharing of genomic segments. These samples included individuals from two-generation pedigrees that had shared haplotypes overlapping regions identified in the extended pedigrees. Eight to nine million 36 base short reads were obtained from each sample. The short reads alignment against the National Cancer Biotechnology Information (NCBI) reference human genome build 36 revealed coverage of 86 to 97% of the designed capture area, with the average read depth over the designed capture area of 30 to 47×.

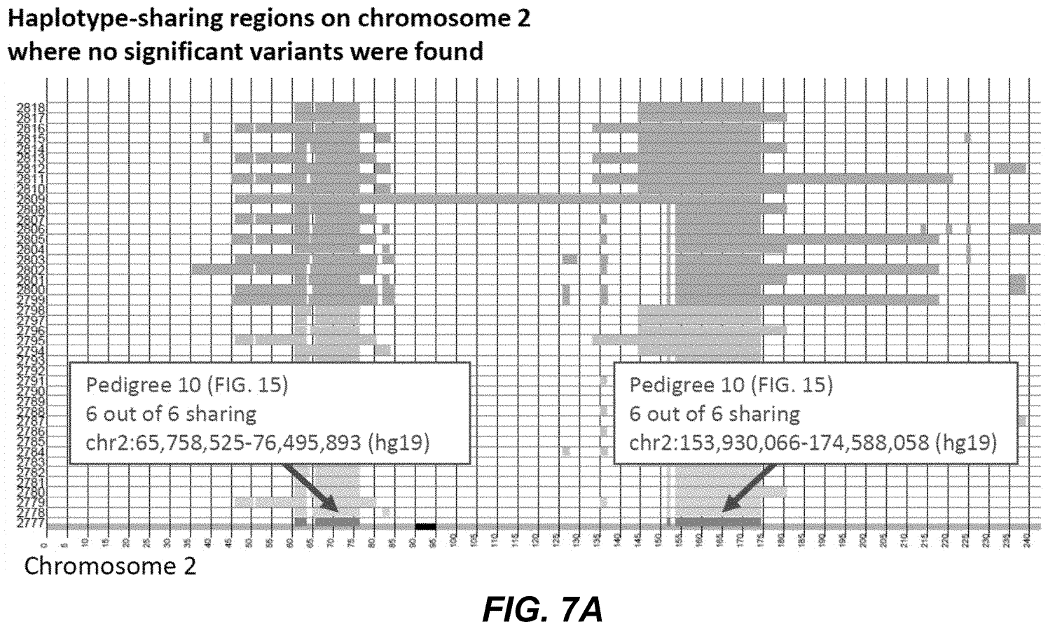

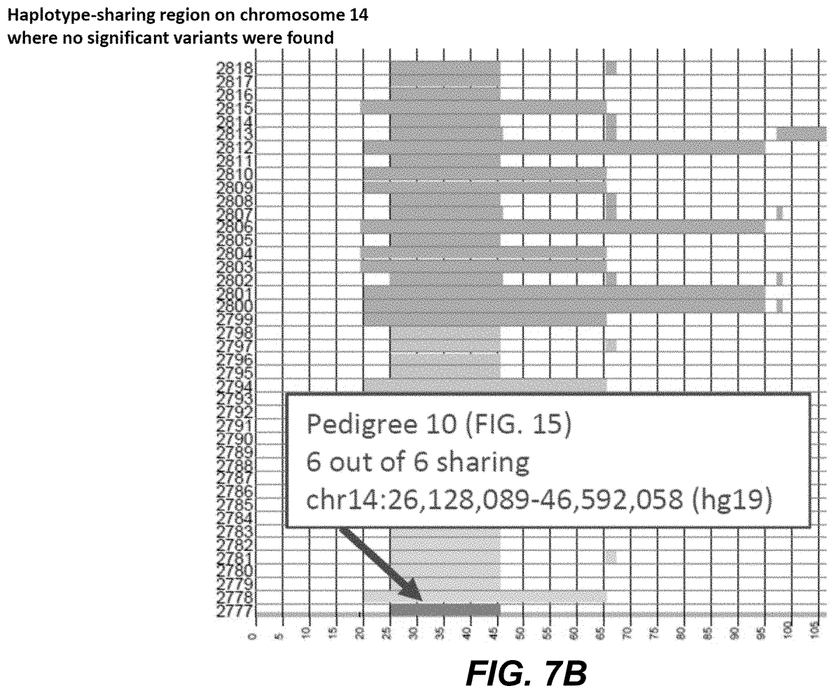

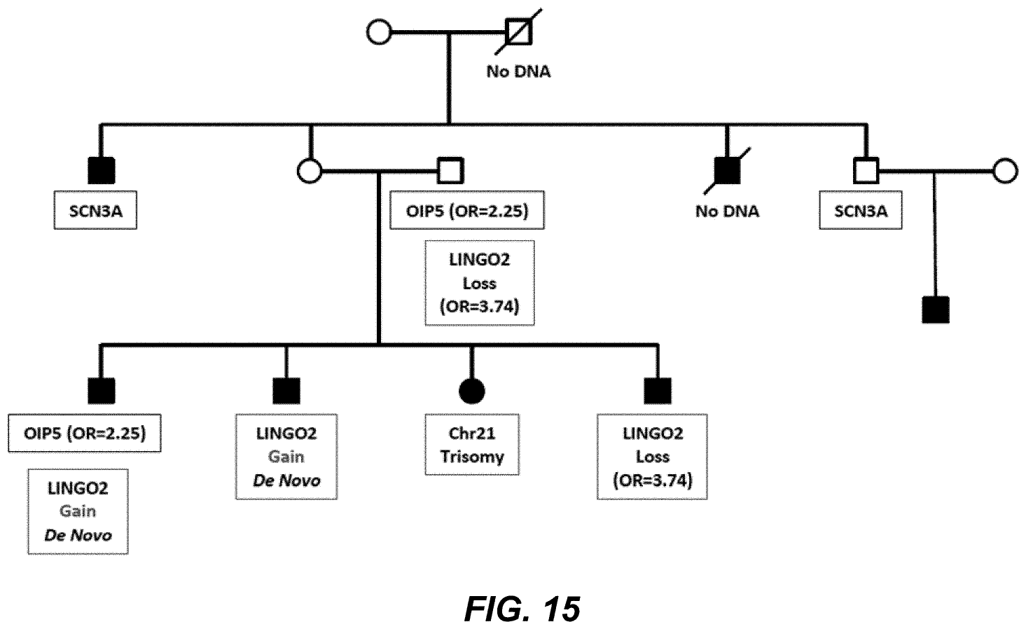

The capture library was constructed in a directional manner, all capture probes represented the same DNA strand, and the library was sequenced only from one direction. Consequently there could be additional variants that were not detected in some of the genes. For example no variants were identified on haplotypes that segregate to all affected individuals in pedigree 10 on chromosomes 2 and 14 (FIG. 7A and FIG. 7B, FIG. 15). Nonetheless, variant calling using the three methods shown in Table 4 identified over 1 million sequence variants called by at least two of the three methods. Analysis using cSNP classifier resulted in the detection of 2,825 SNPs, including 210 nonsense variants, 1,614 non-conservative missense variants, 35 frameshift variants and 966 splice site variants.

A custom microarray was designed to evaluate the variants that were identified by sequencing in order to (1) interrogate the entire set of functional SNPs in the discovery families for validation, and (2) to perform a large scale case/control study to determine if any of the variants identified predisposition genes important to the broad population of children with ASD (FIG. 1). Following array design and manufacture, probes for 2,413 variants were created successfully. Custom microarray experiments on Utah discovery and CHOP case/control samples revealed 584 out of 2,413 variants to be polymorphic. The complete list of polymorphic variants is shown in Table 11. The remaining array probes (1,829 variants) did not detect a non-reference sequence allele. These 1,829 variants thus were interpreted to be false positives due to the variant calling and alignment process of single end sequence data.

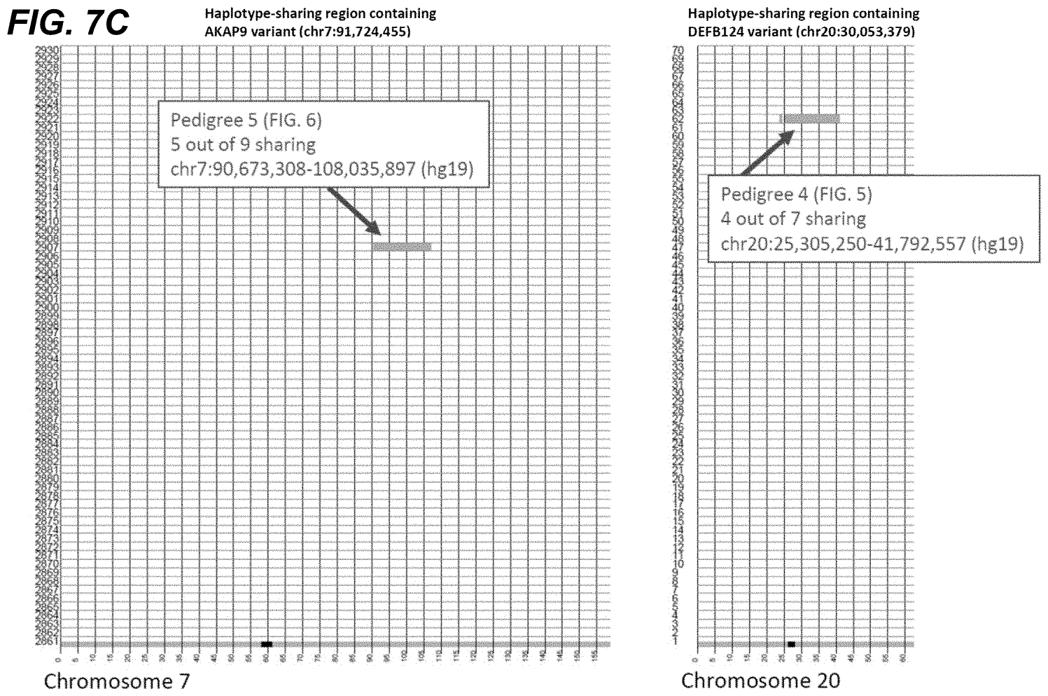

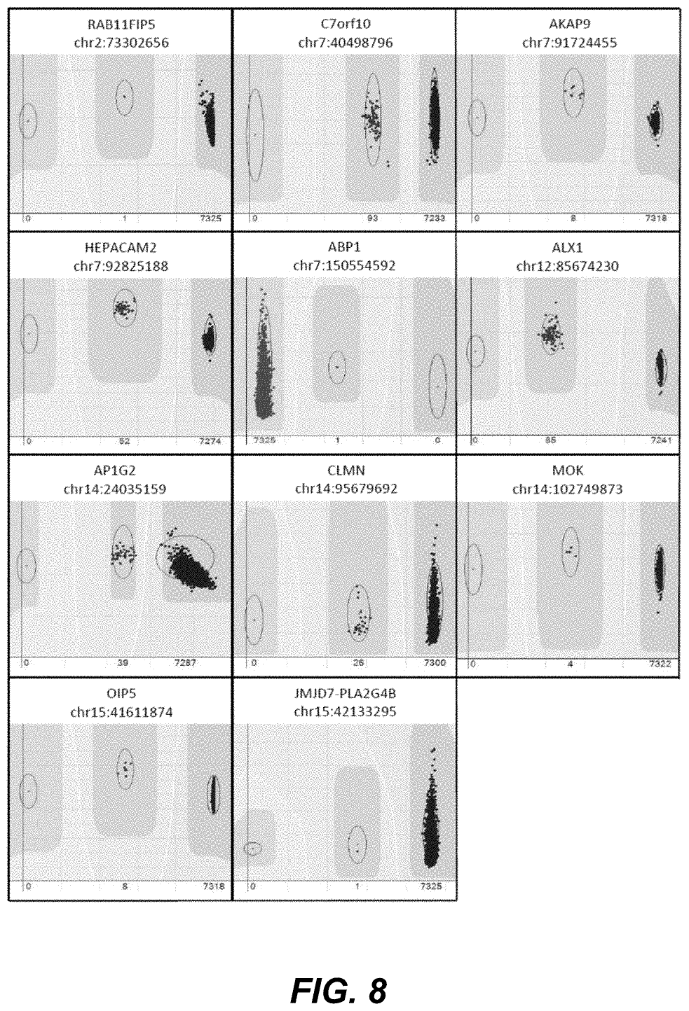

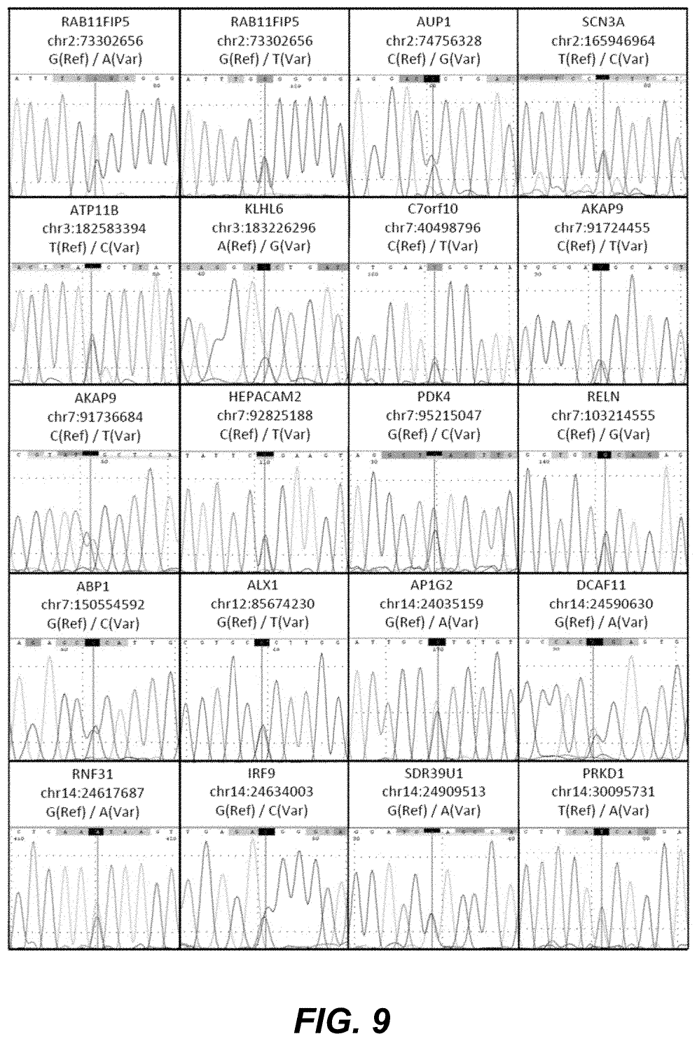

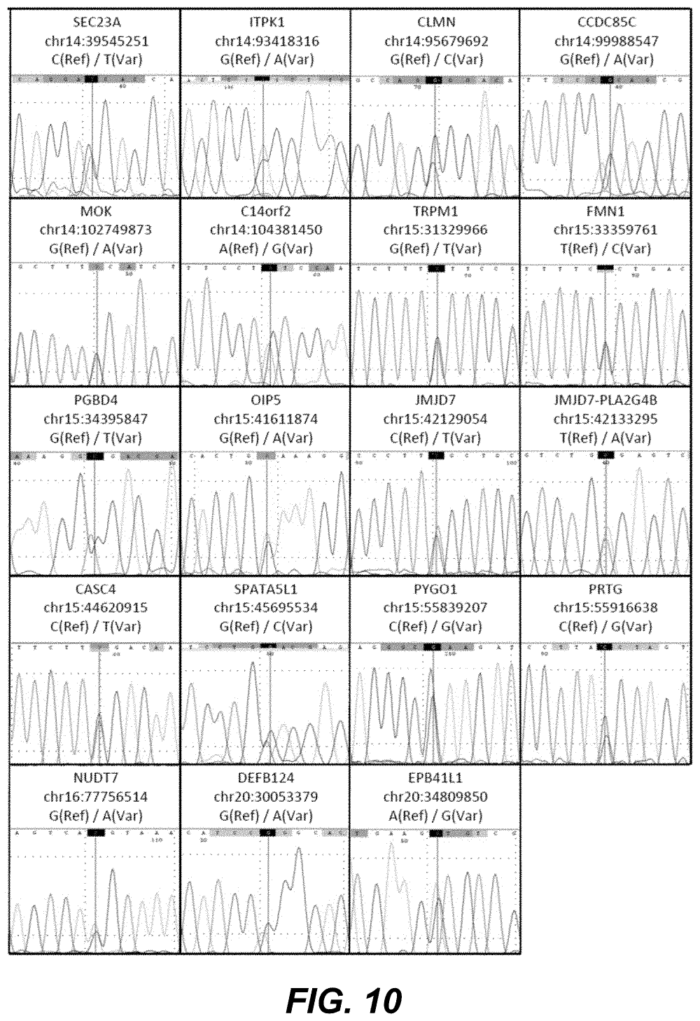

All autosomal SNP variants were tested for association with autism in the case/control study using an allelic association test. Statistical significance of each was assessed using both Fisher's exact test and a chi-squared test. The allelic association test detects any significant result regardless of the direction of the effect. Eleven SNPs (see clustering in FIG. 8) were either unique to cases or had odds ratios (minor allele) greater than 1.5 (Table 6). The variants observed in the case/control study were prioritized for additional work based on an odds ratio cutoff of 1.5. Also included were variants unique to cases. This approach was chosen rather than using p values since these variants were too rare to select based on p values, and for relatively rare diseases odds ratios are approximately equivalent to relative risk values. In addition, 28 SNPs were detected only in the Utah discovery cohort and not in the CHOP cases or controls (Table 7). These 28 SNPs are considered to be potential ASD risk alleles because (i) they are rare or non-existent in the general population and thus could represent “private mutations”, (ii) they may affect protein function, and (iii) they segregate to one or more children with autism in high-risk autism pedigrees. Thus, these 39 SNPs, found in 36 different genes, were characterized as potential autism risk variants. Each of these 39 variants was localized to our targeted regions (Table 5), and 30 of the 39 variants were predicted to be damaging by at least one program embedded in ANNOVAR [35], including SIFT, Polyphen2, LRT and MutationTaster. Details of the analysis of these variants are shown in Table 12. All 39 SNPs were further confirmed by Sanger DNA sequencing of PCR amplicons (see FIG. 9-FIG. 10 for sequence chromatograms). The transcripts used for variant annotation are found in Table 12.

### Segregation of Variants in High-Risk Pedigrees

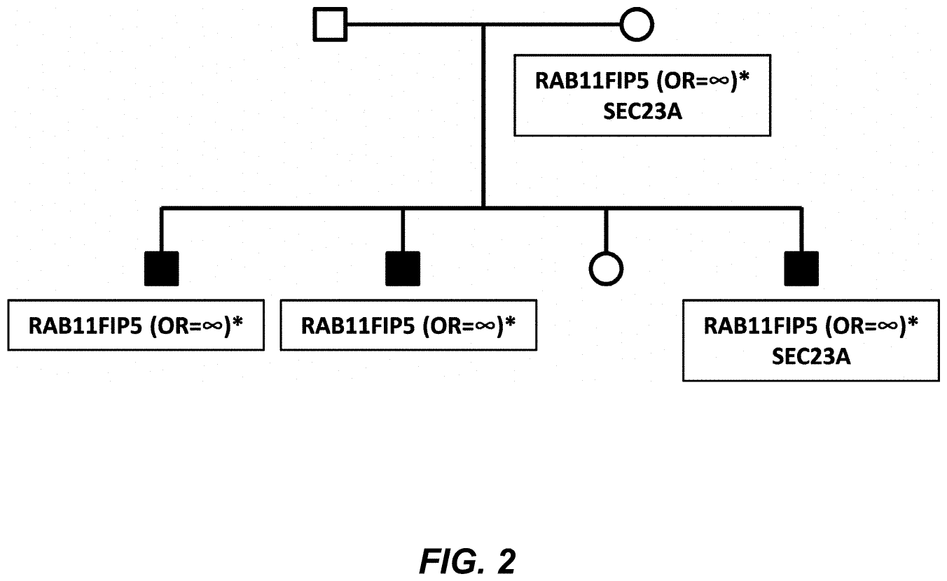

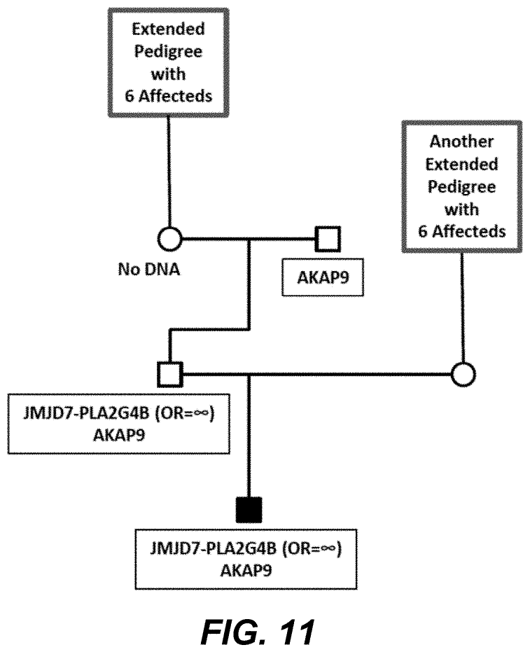

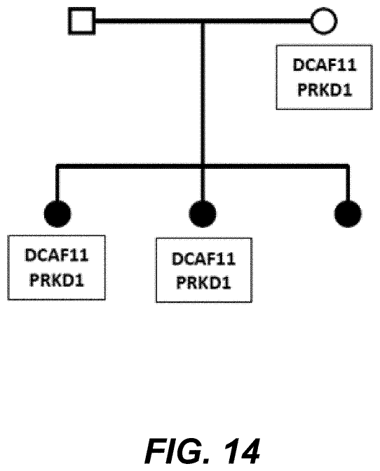

To determine the potential significance of identified variants, the segregation pattern of these variants in the relevant pedigrees was elevated. Potentially detrimental sequence variants were identified in 10 of the 11 pedigrees from which individuals were selected for DNA capture and sequencing. Several of the pedigrees segregated more than one variant, indicating the complexity of the underlying genetics in high-risk ASD pedigrees. Moreover, many of these pedigrees also have CNVs that were identified in previous work [27]. Adding to the genetic complexity, many of these CNVs also segregate to affected individuals. Five families that demonstrate these complex inheritance patterns are shown here (FIG. 2-FIG. 6). Five additional pedigrees with multiple variants are shown in FIG. 11-FIG. 15.

Pedigree 1 (FIG. 2) shows a two-generation family co-segregating a missense variant in RAB11FIP5 (Table 7). This variant is present in the mother and segregates to all three male affected children in the family, and not to the unaffected female child. RAB11FIP5 has previously been implicated as an ASD risk gene based on its disruption by a translocation observed in a 10 year old male child with a diagnosis of pervasive developmental disorder not otherwise specified (PDD-NOS) [41]. The variant detected in pedigree 1 results in a P652L substitution. Proline is conserved at this residue in all of the mammalian RAB11FIP5 genes sequenced to date, suggesting that it is important for protein function. A second individual, with a P652H variant, was detected in the case/control study (Table 6) using the custom microarray. Neither the P652L substitution nor the P652H substitution was observed in the ESP6500, 1000 genomes project or dbSNP137 databases (Table 12). Each of these variants was confirmed by Sanger sequencing (See FIG. 9-FIG. 10 for chromatograms). An additional affected individual of non-European descent, and thus not included in the case/control study, also carried the P652H variant (data not shown). The presence of the P652H variant in an additional individual with autism and not in any controls further supports the likelihood of variants in RAB11FIP5 contributing to autism risk.

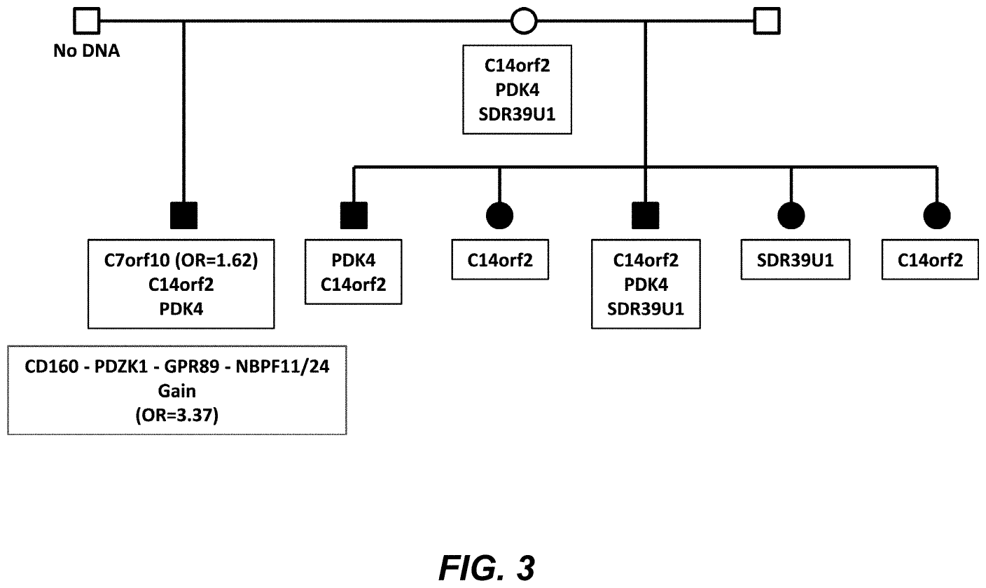

Pedigree 2 (FIG. 3) is a two-generation family with six affected individuals from two fathers. In this pedigree, five of the six affected individuals inherit a variant resulting in an I26T substitution in C14orf2. Two additional sequence variants, one each in the PDK4, and SDR39U1 genes, segregate to three and two affected individuals respectively. In addition, a CNV gain (OR=3.37) described previously [27] is present in one affected individual. The C14orf2 and PDK4 variants were maternally inherited, while the C7orf10 and the CNV were either of paternal origin or occurred as de novo variants. Of the variants detected in this family, only the C7orf10 variant was observed in our case/control study. However, this variant had an odds ratio of 1.62 (95% confidence interval 1.04-2.53), suggesting the possibility for a role in autism predisposition in the general population.

Pedigree 3 (FIG. 4) also is a two generation family, with five male children affected with autism. In this pedigree, four of the five affected individuals exhibit maternal inheritance of an F154L variant in the KLHL6 gene. This A/G nucleotide variant also is found at the first nucleotide of an exon and thus also may affect splicing of the KLHL6 primary transcript. In addition to this variant, three of the five offspring have a paternally inherited D303H missense variant in the SPATA5L1 gene while two of five also have a maternally inherited P238L change in the ITPK1 gene. One affected child does not inherit any of these variants. Of interest, none of the variants observed in this small family were observed in any cases or controls in the population study, demonstrating that they are not common autism predisposition loci.

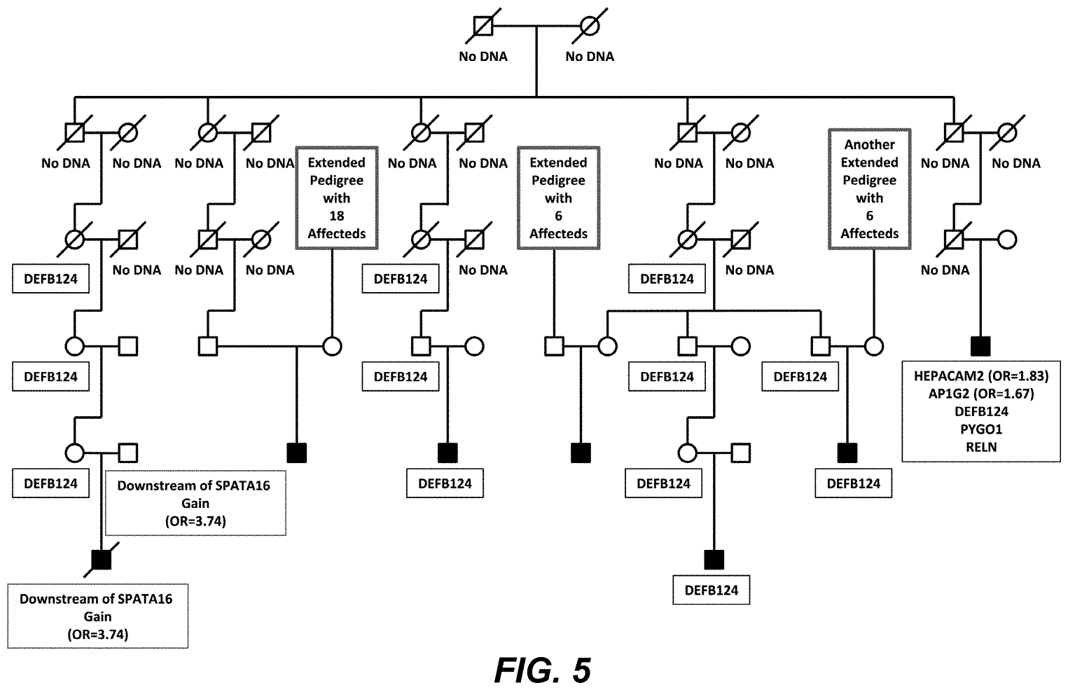

Pedigree 4 (FIG. 5) is a six generation family with an ancestor common to all 7 male children that are affected with autism. These children all are in the fifth or sixth generations of the pedigree. Linkage analysis was performed previously on this family using Affymetrix 10K SNP genotype data [29, 30], and three regions of significant linkage were identified. These include 3q13.2-q13.31, 3q26.31-q27.3, and 20q11.21-q13.12. These three regions also were identified by haplotype sharing in this study (FIG. 5, see FIG. 7C for chromosome 20 haplotype sharing). Four of the seven affected individuals in this family share a P49L variant that is the result of an A/G transition in the DEFB124 gene on chromosome 20q11.21, consistent with the haplotype sharing that we observed (FIG. 7C) and with the published linkage result. This variant was not observed in cases or controls in our population study. One affected individual in this pedigree does not share the DEFB124 variant, but instead has a chromosome 3q gain CNV, inherited from his father, that had an odds ratio of 3.74 in our previous study [27]. The elevated odds ratio suggests that this CNV is an autism risk locus.

Two additional affected individuals in Pedigree 4 do not carry any variant that we detected in our families. However, as indicated in FIG. 5, each of these two individuals is descended from a marry-in spouse with a strong family history of autism, suggesting the possibility of additional undetected variants.

Finally, one affected individual who carries the DEFB124 variant carries variants in the HEPACAM2 gene (odds ratio 1.83 in our population study, Table 6), the AP1G2 gene (odds ratio 1.67, Table 6), the PYGO1 gene and the RELN gene. Neither the RELN variant nor the PYGO1 variant was observed in the case/control study (Table 7). Homozygous or compound heterozygous mutations in RELN are associated with lissencephaly [44,45], but this RELN deletion is the first description of an individual with a developmental phenotype that may be due to haploinsufficiency at this locus.

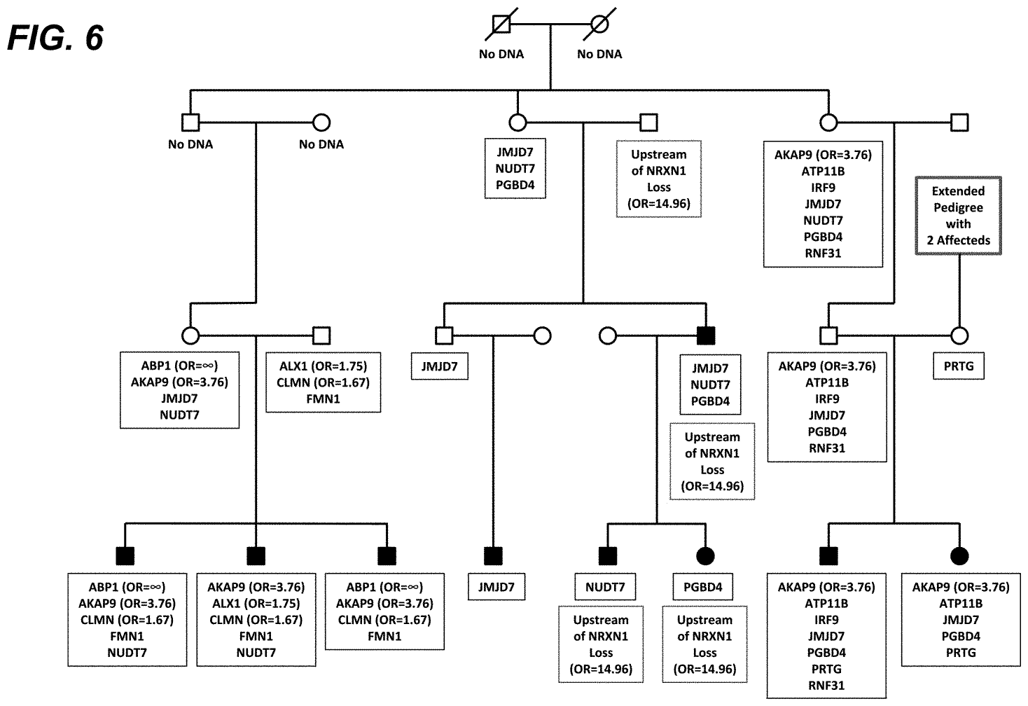

Pedigree 5 (FIG. 6) is a four generation family with nine individuals affected with autism (7 male, 2 female). Two variants are of particular interest in this family. The first is a CNV including the 5′-flanking region of the NRX1α gene. This CNV is inherited from a father who marries into the family in the second generation. This CNV segregates to three of the four descendants of this individual who are diagnosed with autism. An overlapping NRX1□ CNV was shown in our previous work to have an odds ratio of 14.96 [27], consistent with previous work suggesting a role for NRXN1α associated variants in autism, as well as other neurological disorders [46-48]. However, that CNV was shown to extend into the coding region of NRXN1α, while TaqMan CNV analysis demonstrates that the CNV in pedigree 5 did not (data not shown). Thus the significance of the NRXN1α CNV observed in this family is uncertain.

A second variant identified in this family, found on a haplotype shared by all five affected individuals in two branches of the family (FIG. 7C), is a C/T transition in the AKAP9 gene that results in an R3233C missense substitution. None of the individuals in these two branches of the family carry the NRXN1α CNV. The AKAP9 variant was observed in 4/1541 cases and 4/5785 controls in our population study (odds ratio of 3.76, 95% confidence interval 0.94-15.03) (Table 6). A second missense variant in the AKAP9 gene was observed in a single affected individual in a nuclear family (Pedigree 6, FIG. 11). This second AKAP9 variant was not observed in the case/control study (Table 7). The AKAP family of proteins has been suggested to connect different biological pathways that are involved in nervous system development [49].

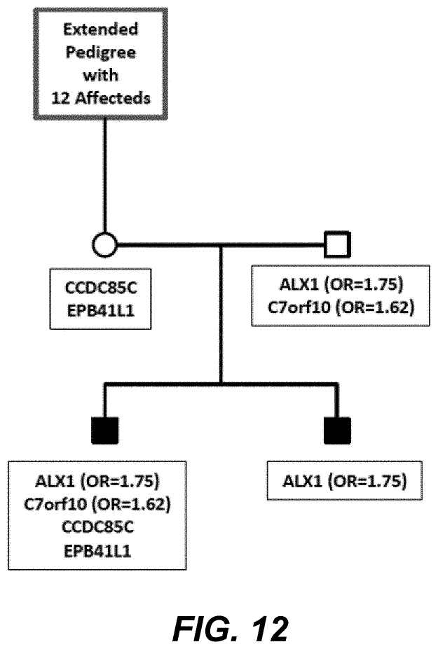

Pedigree 5 also segregates other variants that are inherited by multiple children affected with autism. One branch of the pedigree segregates a G/C transversion in the CLMN gene that results in a P158A missense substitution. This variant yielded an odds ratio of 1.67 (95% confidence interval 0.73-3.84) in our case/control study, suggesting that it is an ASD risk allele. A variant in the ABP1 gene, also the result of a G/C transversion and resulting in an R345P missense substitution, was observed in two affected individuals in a single branch of the family. This variant was maternally inherited and not seen elsewhere in the pedigree. However, this variant was observed in 1/1541 cases and 0/5785 controls in the population study (Table 6) and was not observed in the ESP6500, 1000 Genomes, or dbSNP137 databases (Table 12), indicating that it may be a very rare ASD risk variant. Finally, a G/T transversion in the ALX1 gene that results in an R64L missense substitution was paternally inherited by a single individual. This variant also was seen in pedigree 7 (FIG. 12) and was observed multiple times in our population study (27/1541 cases and 58/5785 controls) yielding an odds ratio of 1.75 (95% confidence interval 1.11-2.77) (Table 6). Expression of this gene also may be increased by a downstream balanced translocation in a family with mental retardation, language delay and microcephaly that segregate with the translocation [50].

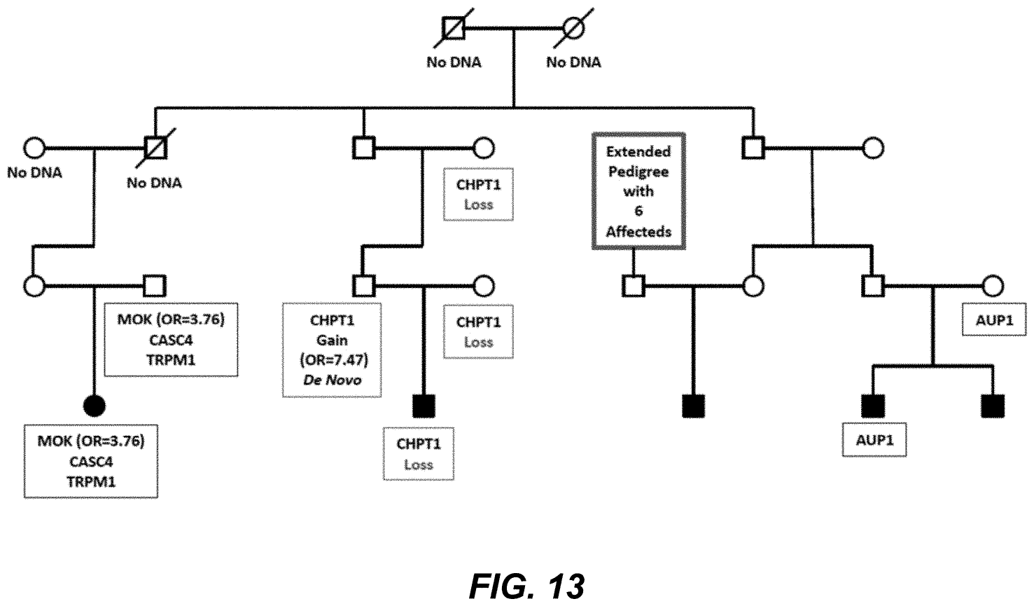

Pedigrees 8-10 are shown in FIG. 13-FIG. 15. One of these pedigrees, pedigree 10, carried two haplotypes (chromosomes 2 and 14) segregating to all six affected individuals (FIG. 7A-FIG. 7B). Sequencing of the genes encompassed by these regions did not identify potential causal variants. This could be due to poor sequence coverage of some portions of the genes. However, sequencing of affected individuals in these families did result in the identification of variants that could be autism risk alleles. One of these variants, a G/A transition that result in a Q22* change in the MOK gene observed in a single affected individual and inherited from her father, was observed in our population study and yielded an odds ratio of 3.76 (95% confidence interval 0.53-26.67) (Table 6). Other variants in pedigrees 8-10 (FIG. 13-FIG. 15), including some only seen in Utah families and others seen in both families and in our population study also were identified. These variants are included in Table 6 and Table 7.

Functional analysis of RAB11FIP

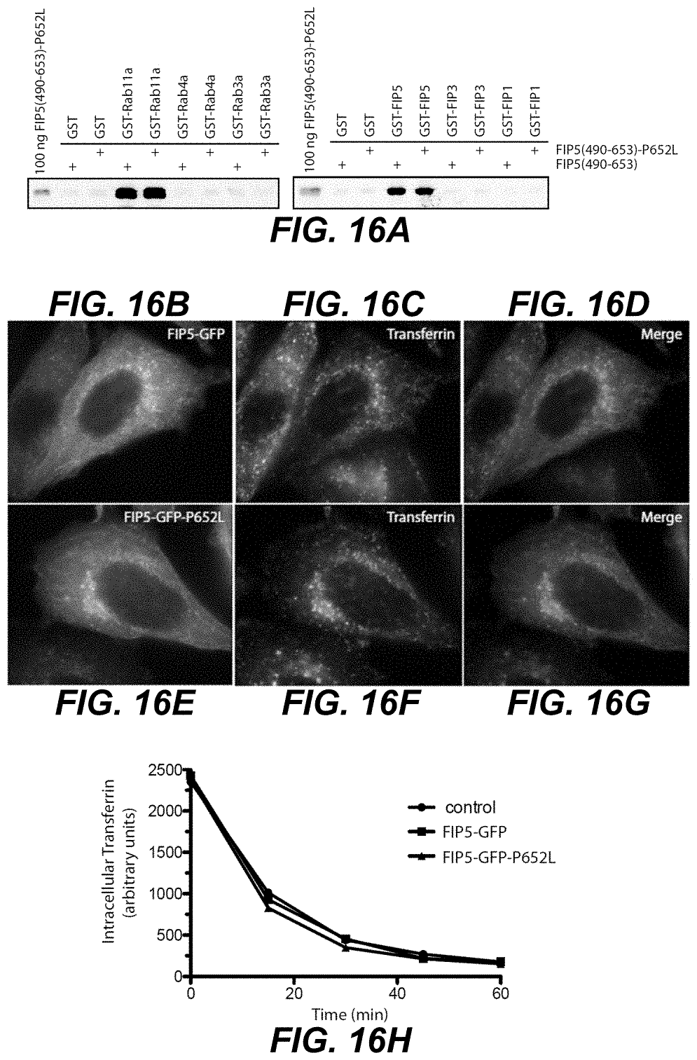

To uncover the functional consequences of the Rab11FIP5-P652L variant, binding of Rab11FIP5 to Rab11. Rab11 is a small monomeric GTPase that mediates Rab11FIP5 recruitment to endocytic membranes and is required for Rab11FIP5 function, was evaluated [41]. As shown in FIG. 16A, the P652L substitution did not affect Rab11FIP5 binding to Rab11, nor did it affect its specificity toward the Rab11 GTPase. It was previously shown that Rab11FIP5 forms homodimers and that its ability to dimerize is also required for Rab11FIP5 cellular functions [41]. Thus, the effect of P652L substitution on Rab11FIP5 ability to dimerize was tested. As shown in FIG. 16B, the Rab11FIP5-P652L mutant was still able to form dimers. Consistent with in vitro binding data, FIP5-GFP-P652L endocytic localization in HeLa cells was also not affected (FIG. 16B-FIG. 16E).

Rab11FIP5 has been reported to function by regulating endocytic recycling [51]. To that end, Rab11FIP5-P652L was tested for a potential effect on recycling of transferrin receptors in HeLa cells. It was found that the P652L substitution did not alter recycling (FIG. 16H). Thus, functional consequences of Rab1FIP5-P652L substitution was not detected, suggesting that core Rab11FIP5 properties are not affected.

A discovery/validation strategy based on identifying inherited genetic variants in two to six generation ASD families was employed, followed by a case/control analysis of those variants in DNA samples from unrelated children with autism and children with normal development to identify familial ASD predisposition genes. Using haplotype analysis shared genomic segments within the families were identified, and DNA sequencing and CNV analysis was used to identify potential causal mutations on those haplotypes. A large case/control study was subsequently employed to determine if any of the variants we identified might play a role in the general population of individuals with ASD.

It was previously shown that identification of CNVs in a family-based discovery cohort could identify copy number variants relevant to the general ASD population [27].

39 SNPs were identified that are likely to affect protein function that have segregation patterns and ASD case allele frequencies suggestive of a role in ASD predisposition. Thirty-one of these variants result in non-conservative amino acid substitutions, five are predicted to affect splicing (3 of these are predicted to affect both splicing and protein coding), and three introduce premature termination codons. Two variants were identified in the AKAP9 gene and the JMJD7 (or the JMJD7-PLA2G4B fusion gene), and two different variants were identified that affect the same amino acid residue in the RAB11FIP5 gene, so collectively these SNPs identify 36 potential ASD risk genes.

With the exception of two-generation families, and consistent with our haplotype sharing results, no sequence variants or CNVs implicated as ASD predisposition loci segregate to all affected individuals in a pedigree. This is consistent with previous genetic studies, which to date have been unable to demonstrate segregation of a single ASD risk locus in an extended family (for example see [52]). In Pedigree 5 (FIG. 6), two independent risk variants, a single nucleotide variant in AKAP9 and a deletion CNV in or near NRXN1, segregate to different branches of the family. Other risk variants also are found in individuals with ASD in this family, including two sequence variants with odds ratios greater than 1.5 in our population study. These results suggests that even in extended families that might be predicted to be segregating a single risk allele with reduced penetrance, multiple risk alleles in different ASD predisposition loci may be necessary. The results further suggest that use of specific inheritance models when evaluating autism genetics in large families should be approached with caution.

Eleven of the autism risk variants that we identified in our high-risk families are further supported by data from our case/control study. Three of these variants each were seen in a single ASD case (out of 1541 total cases) and in none of 5785 controls. Familial variants that we detected in eight additional genes are more common in ASD cases than in controls, and each has an odds ratio greater than 1.5. Although these variants are rare (all have frequencies of <0.01 in our case/control study), their identification in affected individuals in our ASD families and their increased prevalence in unrelated affected individuals support their role as ASD risk loci.

Several intriguing observations resulted from an extensive literature review of the functions and mechanistic actions of each of these 36 genes and their encoded proteins. A number of the genes have been previously linked to autism or other neurological disorders or have known neurological functions (Table 8) (11 out of 36 genes, or 31%). The functions of several other genes belong to pathways often cited as having relevance to autism. These include genes encoding proteins with immunological functions (inflammatory response), and genes encoding proteins important for energy metabolism and mitochondrial function. These groups account for 19 of the 36 genes on the list (53%). Other genes have as yet unexplored functions, can only be linked to functions based on sequence similarity, or have scattered roles in many other cellular or organismal processes, such as cell cycle control, angiogenesis, protein degradation, or metalloproteinase activity.

RAB11FIP

RAB11FIP5 is a member of a family of scaffolding proteins for the RAS GTPase, Rab11. Specifically, RAB11FIP5 has been characterized as a key player in apical endosome recycling, plasma membrane recycling and transcytosis [55,56]. We identified a P652L variant in three affected siblings in a family of six members, in which the mother is an unaffected P652L carrier. An additional variant resulting in a P652H substitution also was detected in 1/1541 Caucasian ASD cases and 0/5785 Caucasian children with normal development (Table 6). These variants modify a conserved proline within the C-terminus of RAB11FIP5.

Heterozygous disruption of RAB11FIP5 was observed previously in a ten year old boy with a balanced translocation [46, XY, t(2;9)(p13;p24)] that disrupts only the RAB11FIP5 gene [41]. This individual has a clinical diagnosis of PDD-NOS, an autism spectrum disorder. This translocation led the authors to suggest that haploinsufficiency of RAB11FIP5 contributes to the subject's ASD [43]. RAB11FIP5 works closely in conjunction with RAB11, and its presence has been detected in both presynaptic and post-synaptic densities where Rab11 plays a key role in determining synaptic strength in long-term depression[57], regulates norepinephrine transporter trafficking [58], carries out synaptic glutamate receptor recycling [59], and regulates dendritic branching in response to BDNF [60,61]. All of these functions have been suggested to be significant contributors to the etiology of ASDs [62,63] and further support the role of mutations in RAB11FIP5 as ASD risk alleles. Affymetrix 250K SNP genotyping and haplotype sharing

AKAP9

AKAP9 is a member of a family of over 50 proteins that serve as scaffolding partners for PKA, its effectors, and phosphorylation targets. AKAP9, also known as Yotiao, is chiefly expressed in the heart and brain, where the encoded protein serves as a scaffold for PKA, protein phosphatase I, NMDA receptors, the heart potassium channel subunit KCNQ1, IP3R1, and specific isoforms of adenylyl cyclase [64-68]. The subcellular localization and assembly of these multimeric protein scaffolds, mediated by AKAPs, are thought to be essential for function, since disruption of the interaction between the AKAP and its effectors leads to a loss of activity. In the case of KCNQ1, loss of interaction between AKAP9 and KCNQ1 leads to a potentially fatal heart condition, long QT syndrome, which also arises in cases with loss of function mutations in KCNQ1 itself [69].

We identified two variants in the AKAP9 gene. These variants result in R3233C and R3832C substitutions in the encoded protein. These two variants were coincident with autism and were found in two unrelated extended ASD pedigrees (FIG. 6, FIG. 11). The R3233C variant was additionally found in our case/control study. A recent meta-study of the genes identified from the five major autism GWAS studies and autism candidate genes arising from alternative methodologies, such as large scale CNV studies, placed AKAPS as a central, integral gene family linking many of the pathways identified by bioinformatics [49]. Given its role in localizing PKA, adenylyl cyclase isoforms and NMDAR in the postsynaptic scaffold, AKAP9 represents a protein that, like its better-characterized counterpart AKAP5, could function in synaptic transmission and plasticity, glutamatergic receptor function regulation and recycling, and dendritic spine morphology [70]

Two of the genes (MOK, TRPM1) containing potential ASD risk alleles were partially or completely encompassed by risk CNVs observed in our previous study [27]. This suggests that the same genes may be affected by different genetic mechanisms with the same or similar phenotypic result. The CNVs containing these genes were both copy number losses. The MOK sequence variant described here was a nonsense change, while the TRPM1 variant was a missense change. These results are consistent with the MOK and TRPM1 effects being due to haploinsufficiency at these two loci.

Although the heritability for autism is quite high, our data show that numerous genetic variants may confer risk to ASD even in a single family. This finding is consistent with the results of a whole genome sequencing study that used both a recessive model and model independent analyses to identify several potential ASD risk variants in an ASD family with two affected individuals [71]. Consistent with the large number of potential ASD risk genes identified to date, none of the genes identified in this single multiplex ASD [71] family overlapped with the genes identified in our study. Our study adds to this complexity by identifying sequence variants in regions of haplotype sharing in 30 high-risk ASD families of 2-6 generations. Our data further demonstrate that in very large multi-generation families, the likelihood of additional risk variants entering the family from individuals who marry into the pedigree is high.

This study is the first to use an empirical approach to identify shared genomic segments, followed by sequence variant detection to identify potential ASD risk variants in a large set of autism families. 584 non-conservative missense, nonsense, frameshift and splice site variants were identified that might predispose to autism in our high-risk families. 39 DNA sequence variants in 36 genes were identified that potentially represent ASD risk genes. Eleven of these variants were observed to have odds ratios greater than 1.5 in a set of 1541 unrelated children with autism and 5785 controls. Three variants, in the RAB11FIP5, ABP1, and JMJD7-PLA2G4B genes, each were observed in a single case and not in any controls. These variants also were not seen in public sequence databases, suggesting that they may be rare causal ASD variants. Twenty-eight additional rare variants were observed only in high-risk ASD families. Collectively these 39 variants identify 36 genes as ASD risk genes. Segregation of sequence variants and of copy number variants previously detected in these families reveals a complex pattern, with only a RAB11FIP5 variant segregating to all affected individuals in one two-generation pedigree. Some affected individuals were found to have multiple potential risk alleles, including sequence variants and CNVs, suggesting that the high incidence of autism in these families could be best explained by variants at multiple loci.

### REFERENCES

1. Rosenberg R E, Law J K, Yenokyan G, McGready J, Kaufmann W E, Law P A: Characteristics and concordance of autism spectrum disorders among 277 twin pairs. Arch Pediatr Adolesc Med. 2009, 163:907-914.
2. Hallmayer J, Cleveland S, Torres A, Phillips J, Cohen B, Torigoe T, Miller J, Fedele A, Collins J, Smith K, Lotspeich L, Croen L A, Ozonoff S, Lajonchere C, Grether J K, Risch N: Genetic Heritability and Shared Environmental Factors Among Twin Pairs With Autism. Arch Gen Psychiatry 2011, 68:1095-1102.
3. Lichtenstein P, Carlstrom E, Råstam M, Gillberg C, Anckarsäter H: The Genetics of Autism Spectrum Disorders and Related Neuropsychiatric Disorders in Childhood. Am J Psychiatry 2010, 167:1357-1363.
4. Ronald A, Hoekstra R A: Autism spectrum disorders and autistic traits: A decade of new twin studies. Am J Med Genet B Neuropsychiatr Genet 2011, 156B:255-274.
5. International Molecular Genetic Study of Autism Consortium (IMGSAC) ( ) A Full Genome Screen for Autism with Evidence for Linkage to a Region on Chromosome 7q. Hum Mol Genet 1998, 7:571-578.
6. International Molecular Genetic Study of Autism Consortium (IMGSAC): A Genomewide Screen for Autism: Strong Evidence for Linkage to Chromosomes 2q, 7q, and 16p. Am J Hum Genet 2001, 69:570-581.
7. Buxbaum J D, Silverman J, Keddache M, Smith C J, Hollander E, Ramoz N, Reichert J G: Linkage analysis for autism in a subset families with obsessive-compulsive behaviors: Evidence for an autism susceptibility gene on chromosome 1 and further support for susceptibility genes on chromosome 6 and 19. Mol Psychiatry 2004, 9:144-150.
8. Iosifov I, Ronemus M, Levy D, Wang Z, Hakker I, Rosenbaum J, Yamrom B, Lee Y-h, Narzisi G, Leotta A, Kendall J, Grabowska E, Ma B, Marks S, Rodgers L, Stepansky A, Troge J, Andrews P, Bekritsky M, Pradhan K, Ghiban E, Kramer M, Parla J, Demeter R, Fulton L L, Fulton R S, Magrini V J, Ye K, Darnell J, Darnell R B, Mardis E R, Wilson R K, Schatz M C, McCombie W R, Wigler M: De Novo Gene Disruptions in Children on the Autistic Spectrum. Neuron 2012, 74(2):285-299.
9. Sanders S J, Murtha M T, Gupta A R, Murdoch J D, Raubeson M J, Willsey A J, Ercan-Sencicek A G, DiLullo N M, Parikshak N N, Stein J L, Walker M F, Ober G T, Teran N A, Song Y, El-Fishawy P, Murtha R C, Choi M, Overton J D, Bjornson R D, Carriero N J, Meyer K A, Bilguvar K, Mane S M, Sestan N, Lifton R P, Günel M, Roeder K, Geschwind D H, Devlin B, State M W: Disruptive de novo point mutations, revealed by whole-exome sequencing, are strongly associated with Autism Spectrum Disorders. Nature 2012, 485(7397):237-241.
10. Neale B M, Kou Y, Liu L, Ma'ayan A, Samocha K E, Sabo A, Lin C F, Stevens C, Wang L S, Makarov V, Polak P, Yoon S, Maguire J, Crawford E L, Campbell N G, Geller E T, Valladares O, Schafer C, Liu H, Zhao T, Cai G, Lihm J, Dannenfelser R, Jabado O, Peralta Z, Nagaswamy U, Muzny D, Reid J G, Newsham I, Wu Y et al.: Patterns and rates of exonic de novo mutations in autism spectrum disorders. Nature 2012 485(7397):242-245.
11. O'Roak B J, Deriziotis P, Lee C, Vives L, Schwartz J J, Girirajan S, Karakoc E, Mackenzie A P, Ng S B, Baker C, Rieder M J, Nickerson D A, Bernier R, Fisher S E, Shendure J, Eichler E E: Exome sequencing in sporadic autism reveals a highly interconnected protein network and extreme locus heterogeneity. Nature 2012, 485(7397):246-250.
12. O'Roak B J, Vives L, Fu W, Egertson J D, Stanaway I B, Phelps I G, Carvill G, Kumar A, Lee C, Ankenman K, Munson J, Hiatt J B, Turner E H, Levy R, O'Day D R, Krumm N, Coe B P, Martin B K, Borenstein E, Nickerson D A, Mefford H C, Doherty D, Akey J M, Bernier R, Eichler E E, Shendure J: Multiplex targeted sequencing identifies recurrently mutated genes in autism spectrum disorders. Science 2012, 338(6114):1619-1622.
13. Lim E T, Raychaudhuri S, Sanders S J, Stevens C, Sabo A, MacArthur D G, Neale B M, Kirby A, Ruderfer D M, Fromer M, Lek M, Liu L, Flannick J, Ripke S, Nagaswamy U, Muzny D, Reid J G, Hawes A, Newsham I, Wu Y, Lewis L, Dinh H, Gross S, Wang L S, Lin C F, Valladares O, Gabriel S B, dePristo M, Altshuler D M, Purcell S M et al.: Rare complete knockouts in humans: population distribution and significant role in autism spectrum disorders. Neuron 2013 77(2):235-242.
14. Yu T W, Chahrour M H, Coulter M E, Jiralerspong S, Okamura-Ikeda K, Ataman B, Schmitz-Abe K, Harmin D A, Adli M, Malik A N, D'Gama A M, Lim E T, Sanders S J, Mochida G H, Partlow J N, Sunu C M, Felie J M, Rodriguez J, Nasir R H, Ware J, Joseph R M, Hill R S, Kwan B Y, Al-Saffar M, Mukaddes N M, Hashmi A, Balkhy S, Gascon G G, Hisama F M, LeClair E, et al.: Using whole-exome sequencing to identify inherited causes of autism. Neuron 2013, 77(2):259-273.
15. Girirajan S, Brkanac Z, Coe B P, Baker C, Vives L, Vu T H, Shafer N, Bernier R, Ferrero G B, Silengo M, Warren S T, Moreno C S, Fichera M, Romano C, Raskind W H, Eichler E E: Relative burden of large CNVs on a range of neurodevelopmental phenotypes. PLoS Genet 2011, 7: e1002334.
16. Sebat J, Lakshmi B, Malhotra D, Troge J, Lese-Martin C, Walsh T, Yamrom B, Yoon S, Krasnitz A, Kendall J, Leotta A, Pai D, Zhang R, Lee Y H, Hicks J, Spence S J, Lee A T, Puura K, Lehtimaki T, Ledbetter D, Gregersen P K, Bregman J, Sutcliffe J S, Jobanputra V, Chung W, Warburton D, King M C, Skuse D, Geschwind D H, Gilliam T C et al.: Strong Association of De Novo Copy Number Mutations with Autism. Science 2007, 316:445-449.
17. Marshall C R, Noor A, Vincent J B, Lionel A C, Feuk L, Skaug J, Shago M, Moessner R, Pinto D, Ren Y, Thiruvahindrapduram B, Fiebig A, Schreiber S, Friedman J, Ketelaars C E, Vos Y J, Ficicioglu C, Kirkpatrick S, Nicolson R, Sloman L, Summers A, Gibbons C A, Teebi A, Chitayat D, Weksberg R, Thompson A, Vardy C, Crosbie V, Luscombe S, Baatj es R, et al.: Structural Variation of Chromosomes in Autism Spectrum Disorder. Am J Hum Genet 2008, 82:477-488.
18. Christian S L, Brune C W, Sudi J, Kumar R A, Liu S, Karamohamed S, Badner J A, Matsui S, Conroy J, McQuaid D, Gergel J, Hatchwell E, Gilliam T C, Gershon E S, Nowak N J, Dobyns W B, Cook E H Jr: Novel Submicroscopic Chromosomal Abnormalities Detected in Autism Spectrum Disorder. Biol Psychiatry 2008, 63:1111-1117.
19. Glessner J T, Wang K, Cai G, Korvatska O, Kim C E, Wood S, Zhang H, Estes A, Brune C W, Bradfield J P, Imielinski M, Frackelton E C, Reichert J, Crawford E L, Munson J, Sleiman P M, Chiavacci R, Annaiah K, Thomas K, Hou C, Glaberson W, Flory J, Otieno F, Garris M, Soorya L, Klei L, Piven J, Meyer K J, Anagnostou E, Sakurai T, et al.: Autism genome-wide copy number variation reveals ubiquitin and neuronal genes. Nature 2009, 459: 569-573.
20. Bucan M, Abrahams B S, Wang K, Glessner J T, Herman E I, Sonnenblick L I, Alvarez Retuerto A I, Imielinski M, Hadley D, Bradfield J P, Kim C, Gidaya N B, Lindquist I, Hutman T, Sigman M, Kustanovich V, Lajonchere C M, Singleton A, Kim J, Wassink T H, McMahon W M, Owley T, Sweeney J A, Coon H, Nurnberger J I, Li M, Cantor R M, Minshew N J, Sutcliffe J S, Cook E H, et al.: Genome-Wide Analyses of Exonic Copy Number Variants in a Family-Based Study Point to Novel Autism Susceptibility Genes. PLoS Genet 2009, 5:e1000536.
21. Pinto D, Pagnamenta A T, Klei L, Anney R, Merico D, Regan R, Conroy J, Magalhaes T R, Correia C, Abrahams B S, Almeida J, Bacchelli E, Bader G D, Bailey A J, Baird G, Battaglia A, Berney T, Bolshakova N, Bolte S, Bolton P F, Bourgeron T, Brennan S, Brian J, Bryson S E, Carson A R, Casallo G, Casey J, Chung B H, Cochrane L, Corsello C, et al.: Functional impact of global rare copy number variation in autism spectrum disorders. Nature 2010, 466:368-372.
22. Szatmari P, Paterson A D, Zwaigenbaum L, Roberts W, Brian J Mapping autism risk loci using genetic linkage and chromosomal rearrangements. Nat Genet 2007, 39:319-328.
23. Sanders S J, Ercan-Sencicek A G, Hus V, Luo R, Murtha M T, Moreno-De-Luca D, Chu S H, Moreau M P, Gupta A R, Thomson S A, Mason C E, Bilguvar K, Celestino-Soper P B, Choi M, Crawford E L, Davis L, Wright N R, Dhodapkar R M, DiCola M, DiLullo N M, Fernandez T V, Fielding-Singh V, Fishman D O, Frahm S, Garagaloyan R, Goh G S, Kammela S, Klei L, Lowe J K, Lund S C, et al.: Multiple recurrent de novo CNVs, including duplications of the 7q11.23 Williams syndrome region, are strongly associated with autism. Neuron 2011, 70:863-885.
24. Weiss L A, Shen Y, Korn J M, Arking D E, Miller D T, Fossdal R, Saemundsen E, Stefansson H, Ferreira M A, Green T, Platt O S, Ruderfer D M, Walsh C A, Altshuler D, Chakravarti A, Tanzi R E, Stefansson K, Santangelo S L, Gusella J F, Sklar P, Wu B L, Daly M J; Autism Consortium: Association between Microdeletion and Microduplication at 16p11.2 and Autism. N Engl J Med 2008, 358:667-675.
25. Morrow E M, Yoo S Y, Flavell S W, Kim T K, Lin Y, Hill R S, Mukaddes N M, Balkhy S, Gascon G, Hashmi A, Al-Saad S, Ware J, Joseph R M, Greenblatt R, Gleason D, Ertelt J A, Apse K A, Bodell A, Partlow J N, Barry B, Yao H, Markianos K, Ferland R J, Greenberg M E, Walsh C A: Identifying Autism Loci and Genes by Tracing Recent Shared Ancestry. Science 2008, 321:218-223.
26. Jacquemont M L, Sanlaville D, Redon R, Raoul O, Cormier-Daire V, Lyonnet S, Amiel J, Le Merrer M, Heron D, de Blois M C, Prieur M, Vekemans M, Carter N P, Munnich A, Colleaux L, Philippe A: Array-based comparative genomic hybridisation identifies high frequency of cryptic chromosomal rearrangements in patients with syndromic autism spectrum disorders. J Med Genet 2006, 43:843-849.
27. Matsunami N, Hadley D, Hensel C H, Christensen G B, Kim C, Frackelton E, Thomas K, da Silva R P, Stevens J, Baird L, Otterud B, Ho K, Varvil T, Leppert T, Lambert C G, Leppert M, Hakonarson H: Identification of Rare Recurrent Copy Number Variants in High-Risk Autism Families and their Prevalence in a Large ASD Population. PLoS One 2013, 8(1):e52239.
28. Allen-Brady K, Robison R, Cannon D, Varvil T, Villalobos M, Pingree C, Leppert M F, Miller J, McMahon W M, Coon H: Genome-wide linkage in Utah autism pedigrees. Mol Psychiatry 2010, 15(10):1006-1015.
29. Coon H, Matsunami N, Stevens J, Miller J, Pingree C, Camp N J, Thomas A, Krasny L, Lainhart J, Leppert M F, McMahon W: Evidence for linkage on chromosome 3q25-27 in a large autism extended pedigree. Hum Hered 2005 60(4):220-226.
30. Allen-Brady K, Miller J, Matsunami N, Stevens J, Block H, Farley M, Krasny L, Pingree C, Lainhart J, Leppert M, McMahon W M, Coon H: A high-density SNP genome-wide linkage scan in a large autism extended pedigree. Mol Psychiatry. 2009 14(6):590-600.
31. BRLMM: an Improved Genotype Calling Method for the GeneChip® Human Mapping 500K Array Set [http://media.affymetrix.com/support/technical/whitepapers/brlmm_whitepaper.pdf]
32. O'Connell J R, Weeks D E: PedCheck: a program for identification of genotype incompatibilities in linkage analysis. Am J Hum Genet 1998, 63(1):259-266.
33. Arrington C B, Bleyl S B, Matsunami N, Bowles N E, Leppert T I, Demarest B L, Osborne K, Yoder B A, Byrne J L, Schiffman J D, Null D M, DiGeronimo R, Rollins M, Faix R, Comstock J, Camp N J, Leppert M F, Yost H J, Brunelli L: A family-based paradigm to identify candidate chromosomal regions for isolated congenital diaphragmatic hernia. Am J Med Genet A. 2012, 158A(12):3137-47.
34. Langmead B, Trapnell C, Pop M, Salzberg S L: Ultrafast and memory-efficient alignment of short DNA sequences to the human genome. Genome Biol 2009, 10(3):R25.
35. Li H, Ruan J, Durbin R: Mapping short DNA sequencing reads and calling variants using mapping quality scores. Genome Res 2008, 18(11):1851-1858.
36. Hillier L W, Marth G T, Quinlan A R, Dooling D, Fewell G, Barnett D, Fox P, Glasscock J I, Hickenbotham M, Huang W, Magrini V J, Richt R J, Sander S N, Stewart D A, Stromberg M, Tsung E F, Wylie T, Schedl T, Wilson R K, Mardis E R: Whole-genome sequencing and variant discovery in C. elegans. Nat. Methods 2008, 5(2):183-188.
37. Yandell M, Huff C, Hu H, Singleton M, Moore B, Xing J, Jorde L B, Reese M G: A probabilistic disease-gene finder for personal genomes. Genome Res 2011, 21(9):1529-1542.
38. Wang K, Li M, Hakonarson H: ANNOVAR: functional annotation of genetic variants from high-throughput sequencing data. Nucleic Acids Res. 2010, 38(16):e164.
39. Kent W J, Sugnet C W, Furey T S, Roskin K M, Pringle T H, Zahler A M, Haussler D: The human genome browser at UCSC. Genome Res. 2002, 12(6):996-1006.
40. Meyer L R, Zweig A S, Hinrichs A S, Karolchik D, Kuhn R M, Wong M, Sloan C A, Rosenbloom K R, Roe G, Rhead B, Raney B J, Pohl A, Malladi V S, Li C H, Lee B T, Learned K, Kirkup V, Hsu F, Heitner S, Harte R A, Haeussler M, Guruvadoo L, Goldman M, Giardine B M, Fujita P A, Dreszer T R, Diekhans M, Cline M S, Clawson H, Barber G P, et al.: The UCSC Genome Browser database: extensions and updates 2013. Nucleic Acids Res 2013, 41(Database issue):D64-69.
41. Junutula J R, Schonteich E, Wilson G M, Peden A A, Scheller R H, Prekeris R: Molecular characterization of Rab11 interactions with members of the family of Rab11-interacting proteins. J Biol Chem 2004, 279(32):33430-33437.
42. Peden A A, Schonteich E, Chun J, Junutula J R, Scheller R H, Prekeris R: The RCP-Rab11 complex regulates endocytic protein sorting. Mol Biol Cell 2004, 15(8):3530-3541.
43. Roohi J, Tegay D H, Pomeroy J C, Burkett S, Stone G, Stanyon R, Hatchwell E: A de novo apparently balanced translocation [46,XY,t(2;9)(p13;p24)] interrupting RAB11FIP5 identifies a potential candidate gene for autism spectrum disorder. Am J Med Genet B Neuropsychiatr Genet 2008, 147B(4):411-417.
44. Hong S E, Shugart Y Y, Huang D T, Shahwan S A, Grant P E, Hourihane J O, Martin N D, Walsh C A: Autosomal recessive lissencephaly with cerebellar hypoplasia is associated with human RELN mutations. Nat Genet 2000, 26(1):93-96.
45. Zaki M, Shehab M, El-Aleem A A, Abdel-Salam G, Koeller H B, Ilkin Y, Ross M E, Dobyns W B, Gleeson J G: Identification of a novel recessive RELN mutation using a homozygous balanced reciprocal translocation. Am J Med Genet A 2007, 143A(9):939-944.
46. Bella F, Bruno D L, Eriksson M, van Ravenswaaij-Arts C, Stark Z, Dijkhuizen T, Gerkes E, Gimelli S, Ganesamoorthy D, Thuresson A C, Labalme A, Till M, Bilan F, Pasquier L, Kitzis A, Dubourgm C, Rossi M, Bottani A, Gagnebin M, Sanlaville D, Gilbert-Dussardier B, Guipponi M, van Haeringen A, Kriek M, Ruivenkamp C, Antonarakis S E, Anderlid B M, Slater H R, Schoumans J: Molecular and clinical characterization of 25 individuals with exonic deletions of NRXN1 and comprehensive review of the literature. Am J Med Genet B Neuropsychiatr Genet 2013, 162B(4):388-403.
47. Nag A, Bochukova E G, Kremeyer B, Campbell D D, Muller H, Valencia-Duarte A V, Cardona J, Rivas I C, Mesa S C, Cuartas M, Garcia J, Bedoya G, Cornejo W, Herrera L D, Romero R, Fournier E, Reus V I, Lowe T L, Farooqi I S; Tourette Syndrome Association International Consortium for Genetics, Mathews C A, McGrath L M, Yu D, Cook E, Wang K, Scharf J M, Pauls D L, Freimer N B, Plagnol V, Ruiz-Linares A: CNV analysis in Tourette syndrome implicates large genomic rearrangements in COL8A1 and NRXN1. PLoS One 2013, 8(3):e59061.
48. Schaaf C P, Boone P M, Sampath S, Williams C, Bader P I, Mueller J M, Shchelochkov O A, Brown C W, Crawford H P, Phalen J A, Tartaglia N R, Evans P, Campbell W M, Tsai A C, Parsley L, Grayson S W, Scheuerle A, Luzzi C D, Thomas S K, Eng P A, Kang S H, Patel A, Stankiewicz P, Cheung S W: Phenotypic spectrum and genotype-phenotype correlations of NRXN1 exon deletions. Eur J Hum Genet 2012, 20(12):1240-1247.
49. Poelmans G, Franke B, Pauls D L, Glennon J C, Buitelaar J K: AKAPs integrate genetic findings for autism spectrum disorders. Transl Psychiatry 2013, 3:e270.
50. Liao H M, Fang J S, Chen Y J, Wu K L, Lee K F, Chen C H: Clinical and molecular characterization of a transmitted reciprocal translocation t(1;12)(p32.1;q21.3) in a family co-segregating with mental retardation, language delay, and microcephaly. BMC Med Genet 2011, 12:70.
51. Schonteich E, Wilson G M, Burden J, Hopkins C R, Anderson K, Goldenring J R, Prekeris R: The Rip11/Rab11-FIP5 and kinesin II complex regulates endocytic protein recycling. J Cell Sci 2008, 121(Pt 22):3824-3833.
52. Kilpinen H, Ylisaukko-oja T, Rehnstrom K, Gaal E, Turunen J A, Kempas E, von Wendt L, Varilo T, Peltonen L: Linkage and linkage disequilibrium scan for autism loci in an extended pedigree from Finland. Hum Mol Genet 2009, 18(15):2912-2921.
53. Kim Y S, Leventhal B L, Koh Y J, Fombonne E, Laska E, Lim E C, Cheon K A, Kim S J, Kim Y K, Lee H, Song D H, Grinker R R: Prevalence of autism spectrum disorders in a total population sample. Am J Psychiatry 2011, 168(9):904-912.
54. Center for Disease Control and Prevention. [http://www.cdc.gov/ncbddd/autism/data.html]
55. Prekeris R, Klumperman J, Scheller R H: A Rab11/Rip11 protein complex regulates apical membrane trafficking via recycling endosomes. Mol Cell 2000, 6(6):1437-1448.
56. Hales C M, Griner R, Hobdy-Henderson K C, Dorn M C, Hardy D, Kumar R, Navarre J, Chan E K, Lapierre L A, Goldenring J R: Identification and characterization of a family of Rab11-interacting proteins. J Biol Chem 2001, 276(42):39067-75.
57. Fernandez-Monreal M, Brown T C, Royo M, Esteban J A: The balance between receptor recycling and trafficking toward lysosomes determines synaptic strength during long-term depression. J Neurosci. 2012, 32(38):13200-13205.
58. Matthies H J, Moore J L, Saunders C, Matthies D S, Lapierre L A, Goldenring J R, Blakely R D, Galli A: Rab11 supports amphetamine-stimulated norepinephrine transporter trafficking. J Neurosci 2010, 30(23):7863-7877.
59. van der Sluijs P, Hoogenraad C C: New insights in endosomal dynamics and AMPA receptor trafficking. Semin Cell Dev Bio. 2011, 22(5):499-505.
60. Park M, Salgado J M, Ostroff L, Helton T D, Robinson C G, Harris K M, Ehlers M D: Plasticity-induced growth of dendritic spines by exocytic trafficking from recycling endosomes. Neuron 2006, 52(5):817-830.
61. Lazo O M, Gonzalez A, Ascalio M, Kuruvilla R, Couve A, Bronfman F C: BDNF regulates Rab11-mediated recycling endosome dynamics to induce dendritic branching. J Neurosci 2013, 33(14):6112-6122.
62. Penzes P, Cahill M E, Jones K A, VanLeeuwen J E, Woolfrey K M: Dendritic spine pathology in neuropsychiatric disorders. Nat Neurosci 2011, 14(3):285-293.
63. Ebert D H, Greenberg M E: Activity-dependent neuronal signalling and autism spectrum disorder. Nature 2013, 493(7432):327-337.
64. Piggot J, Shirinyan D, Shemmassian S, Vazirian S, Alarcon M: Neural systems approaches to the neurogenetics of autism spectrum disorders. Neuroscience 2009, 164(1):247-256.
65. Lin L, Sun W, Kung F, Dell'Acqua M L, Hoffman D A: AKAP79/150 impacts intrinsic excitability of hippocampal neurons through phospho-regulation of A-type K+ channel trafficking. J Neurosci 2011, 31(4):1323-1332.
66. Westphal R S, Tavalin S J, Lin J W, Alto N M, Fraser I D, Langeberg L K, Sheng M, Scott J D: Regulation of NMDA receptors by an associated phosphatase-kinase signaling complex. Science 1999, 285(5424):93-96.
67. Marx S O, Kurokawa J, Reiken S, Motoike H, D'Armiento J, Marks A R, Kass R S. Requirement of a macromolecular signaling complex for beta adrenergic receptor modulation of the KCNQ1-KCNE1 potassium channel. Science 2002, 295(5554):496-499.
68. Tu H, Tang T S, Wang Z, Bezprozvanny I: Association of type 1 inositol 1,4,5-trisphosphate receptor with AKAP9 (Yotiao) and protein kinase A. J Biol Chem. 2004, 279(18):19375-19382.
69. Chen L, Marquardt M L, Tester D J, Sampson K J, Ackerman M J, Kass R S. Mutation of an A-kinase-anchoring protein causes long-Q T syndrome. Proc Natl Acad Sci USA 2007, 104(52):20990-20995.
70. Keith D J, Sanderson J L, Gibson E S, Woolfrey K M, Robertson H R, Olszewski K, Kang R, El-Husseini A, Dell'acqua M L: Palmitoylation of A-kinase anchoring protein 79/150 regulates dendritic endosomal targeting and synaptic plasticity mechanisms. J Neurosci 2012, 32(21):7119-7136.
71. Shi L, Zhang X, Golhar R, Otieno F G, He M, Hou C, Kim C, Keating B, Lyon G J, Wang K, Hakonarson H: Whole-genome sequencing in an autism multiplex family. Mol Autism 2013, 4(1):8.
72. Chen C P, Lin S P, Chen S R, Chen Y J, Tsai F J, Wu P C, Wang W: Array-CGH detection of a de novo 2.8 Mb deletion in 2q24.2-->q24.3 in a girl with autistic features and developmental delay. Eur J Med Genet 2010, 53(4):217-220.
73. Uz E, Alanay Y, Aktas D, Vargel I, Gucer S, Tuncbilek G, von Eggeling F, Yilmaz E, Deren O, Posorski N, Ozdag H, Liehr T, Balci S, Alikasifoglu M, Wollnik B, Akarsu N A. Disruption of ALX1 causes extreme microphthalmia and severe facial clefting: expanding the spectrum of autosomal-recessive ALX-related frontonasal dysplasia. Am J Hum Genet. 2010, 86(5):789-96.
74. Mori N, Kuwamura M, Tanaka N, Hirano R, Nabe M, Ibuki M, Yamate J: Ccdc85c encoding a protein at apical junctions of radial glia is disrupted in hemorrhagic hydrocephalus (hhy) mice. Am J Pathol 2012, 180(1):314-327.
75. Hamdan F F, Gauthier J, Araki Y, Lin D T, Yoshizawa Y, Higashi K, Park A R, Spiegelman D, Dobrzeniecka S, Piton A, Tomitori H, Daoud H, Massicotte C, Henrion E, Diallo O; S2D Group, Shekarabi M, Marineau C, Shevell M, Maranda B, Mitchell G, Nadeau A, D'Anjou G, Vanasse M, Srour M, Lafrenière R G, Drapeau P, Lacaille J C, Kim E et al.: Excess of de novo deleterious mutations in genes associated with glutamatergic systems in nonsyndromic intellectual disability. Am Ilium Genet 2011, 88(3):306-316.
76. Majerus P W, Wilson D B, Zhang C, Nicholas P J, Wilson M P: Expression of inositol 1,3,4-trisphosphate 5/6-kinase (ITPK1) and its role in neural tube defects. Adv Enzyme Regul 2010, 50(1):365-372.
77. Marzinke M A, Clagett-Dame M: The all-trans retinoic acid (atRA)-regulated gene Calmin (Clmn) regulates cell cycle exit and neurite outgrowth in murine neuroblastoma (Neuro2a) cells. Exp Cell Res 2012, 318(1):85-93.
78. Wong Y H, Lu A C, Wang Y C, Cheng H C, Chang C, Chen P H, Yu J Y, Fann M J: Protogenin defines a transition stage during embryonic neurogenesis and prevents precocious neuronal differentiation. J Neurosci 2010, 30(12):4428-4439.
79. Ghosh M, Loper R, Gelb M H, Leslie C C: Identification of the expressed form of human cytosolic phospholipase A2beta (cPLA2beta): cPLA2beta3 is a novel variant localized to mitochondria and early endosomes. J Biol Chem 2006, 281(24):16615-16624.
80. Sherman E A, Strauss K A, Tortorelli S, Bennett M J, Knerr I, Morton D H, Puffenberger E G: Genetic mapping of glutaric aciduria, type 3, to chromosome 7 and identification of mutations in c7orf10. Am J Hum Genet 2008, 83(5):604-609.
81. Korotchkina L G, Patel M S: Site specificity of four pyruvate dehydrogenase kinase isoenzymes toward the three phosphorylation sites of human pyruvate dehydrogenase. J Biol Chem 2001, 276(40):37223-37229.
82. Meyer B, Wittig I, Trifilieff E, Karas M, Schagger H: Identification of two proteins associated with mammalian ATP synthase. Mol Cell Proteomics 2007, 6(10):1690-1699.
83. Jarczak J, Kościuczuk E M, Lisowski P, Strzalkowska N, Jóźwik A, Horbańczuk J, Krzyżewski J, Zwierzchowski L, Bagnicka E: Defensins: Natural component of human innate immunity. Hum Immunol 2013, 74(9):1069-1079.
84. Holweg A, Schnare M, Gessner A: The bactericidal/permeability-increasing protein (BPI) in the innate defense of the lower airways. Biochem Soc Trans 2011, 39(4):1045-1050.
85. Tokunaga F, Iwai K: Linear ubiquitination: a novel NF-κB regulatory mechanism for inflammatory and immune responses by the LUBAC ubiquitin ligase complex. Endocr J 2012, 59(8):641-652.
86. Nguyen H, Hiscott J, Pitha P M: The growing family of interferon regulatory factors. Cytokine Growth Factor Rev. 1997, 8(4):293-312.

TABLE 4
Sequence alignment and variant detection methods

| | Alignment and Assembly | Sequence Variant Detection |
|---|---|---|
| Method 1 | Bowtie | Maq |
| Method 2 | MOSAIK | GigaBayes |
| Method 3 | CLC Bio Genomics | CLC Bio Genomics |
| | Workbench (CLC Bio Inc.) | Workbench (CLC Bio Inc.) |

TABLE 5
Chromosomal regions selected for sequencing based on haplotype sharing. Where
multiple numbers are given, multiple families shared overlapping haplotypes.

| 18 Shared Haplotype | | | | Affecteds Sharing |
|---|---|---|---|---|
| Regions | Chr | Location (hg18) | Location (hg19) | Haplotype |
| 2p14-p12 | 2 | 65612029-76349401 | 65758525-76495893 | 6 of 6 |
| 2q23-q31 | 2 | 153638312-174296304 | 153930066-174588058 | 6 of 6 |
| 2q37 | 2 | 231435643-238617145 | 231727399-238952406 | 5 of 7 |
| 3q13 | 3 | 111604019-112685490 | 110121329-111202800 | 4 of 7 |
| 3q26-q27 | 3 | 174594938-185701563 | 173112244-184218869 | 4 of 7, 4 of 4 |
| 4q28-q31 | 4 | 137362554-141629142 | 137143104-141409692 | 6 of 6 |
| 7p21 | 7 | 7381742-11861952 | 7415217-11895427 | 4 of 4, 4 of 6 |
| 7p14 | 7 | 36090817-41521542 | 36124292-41555017 | 4 of 7 |
| 7q21-q31 | 7 | 90511244-107823133 | 90673308-108035897 | 5 of 8* |
| 7q35-36 | 7 | 142750349-151152511 | 143040227-151521578 | 4 of 6 |
| 12q21 | 12 | 76119990-77788028 | 77595859-79263897 | 5 of 7 |
| 12q21 | 12 | 79689788-87939487 | 81165657-89415356 | 5 of 8 |
| 14q11-q21 | 14 | 22912579-45661808 | 23842739-46592058 | 3 of 4, 6 of 6 |
| 14q32 | 14 | 92331535-103509782 | 93261782-104440029 | 4 of 4 |
| 15q12-q21 | 15 | 24339787-43759484 | 26788694-45972192 | 3 of 4, 4 of 6, 5 of 8 |
| 16q22-23 | 16 | 73415053-77780513 | 74857552-79223012 | 4 of 7, 5 of 6, 3 of 4 |
| 20p11-q13 | 20 | 25253250-41225971 | 25305250-41792557 | 4 of 7 |
| 20q13 | 20 | 49062886-57757418 | 49629479-58324023 | 5 of 6, 5 of 6 |

*Indicates a family where a ninth affected individual was later shown not to share the same haplotype.

TABLE 6
sequence variants identified in families and observed in the case/control study.

| | | | | | | Odds | Odds | | | | | |
|---|---|---|---|---|---|---|---|---|---|---|---|---|
| | | | | | | Ratio | Ratio | | | | | |
| | | | | | Odds | 95% | 95% | | | | | |
| Variant | | | | | Ratio | Lower | Upper | | Het. | | W.T. | |
| (Ref/ | | Coordinate | Fisher's | Chi- | (Minor | Confidence | Confidence | Het. | Con- | W.T. | Con- | |
| Obs) | Gene | (hg19) | Exact P | Squared P | Allele) | Bound | Bound | Cases | trols | Cases | trols | Variant |
| G/T | RAB11FIP5 | chr2: 73302656 | 2.10E−01 | 0.052671 | infinite | N/A | N/A | 1 | 0 | 1540 | 5785 | P652H |
| G/C | ABP1 | chr7: 150554592 | 2.10E−01 | 0.052671 | infinite | N/A | N/A | 1 | 0 | 1540 | 5785 | R345P |
| T/A | JMJD7- | chr15: 42133295 | 2.10E−01 | 0.052671 | infinite | N/A | N/A | 1 | 0 | 1540 | 5785 | splice |
| | PLA2G4B | | | | | | | | | | | site |
| C/T | C7orf10 | chr7: 40498796 | 4.02E−02 | 0.03 | 1.62 | 1.04 | 2.5319729 | 28 | 65 | 1513 | 5720 | R288W, |
| | | | | | | | | | | | | splice site |
| C/T | AKAP9 | chr7: 91724455 | 6.62E−02 | 0.04 | 3.76 | 0.94 | 15.03362 | 4 | 4 | 1537 | 5781 | R3233C |
| C/T | HEPACAM2 | chr7: 92825188 | 5.84E−02 | 0.04 | 1.83 | 1.02 | 3.2674134 | 17 | 35 | 1524 | 5750 | G398R |
| G/T | ALX1 | chr12: 85674230 | 2.22E−02 | 0.01 | 1.75 | 1.11 | 2.7742452 | 27 | 58 | 1514 | 5727 | R64L |
| G/A | AP1G2 | chr14: 24035159 | 1.66E−01 | 0.14 | 1.67 | 0.85 | 3.3018168 | 12 | 27 | 1529 | 5757 | R99C |
| G/C | CLMN | chr14: 95679692 | 2.29E−01 | 0.22 | 1.67 | 0.73 | 3.8448629 | 8 | 18 | 1533 | 5767 | P158A |
| G/A | MOK | chr14: 102749873 | 1.97E−01 | 0.16 | 3.76 | 0.53 | 26.67471 | 2 | 2 | 1539 | 5783 | Q22* |
| G/A | OIP5 | chr15: 41611874 | 3.77E−01 | 0.25 | 2.25 | 0.54 | 9.4355661 | 3 | 5 | 1538 | 5780 | S165F |

*Indicates a mutation that results in a nonsense codon. Standard single letter amino acid designations are used.

TABLE 7
Sequence variants observed only in high-risk ASD families.

| | | | | Tested | Affecteds | | |
|---|---|---|---|---|---|---|---|
| Variant | | Coordinate | Pedigree | Affecteds | with | Coding | |
| (Ref/Obs) | Gene | (hg19) | Structure | in Pedigree | Variant | Change | ESP6500_ALL |
| G/A | RAB11FIP5 | chr2: 73302656 | 2- | 3 | 3 | P652L | |
| | | | Generation | | | | |
| C/G | AUP1 | chr2: 74756328 | Extended | 5 | 1 | R90S | |
| T/C | SCN3A | chr2: 165946964 | Extended | 6 | 1 | E1851G | |
| T/C | ATP11B | chr3: 182583394 | Extended | 9 | 2 | S451P | |
| A/G | KLHL6 | chr3: 183226296 | 2- | 5 | 4 | F154L, | |
| | | | Generation | | | splicing | |
| C/T | AKAP9 | chr7: 91736684 | Extended | 7 | 1 | R3832C | 0.000154 |
| G/C | PDK4 | chr7: 95215047 | 2- | 6 | 3 | S381* | |
| | | | Generation | | | | |
| C/G | RELN | chr7: 103214555 | Extended | 7 | 1 | D1499H | 0.000231 |
| G/A | DCAF11 | chr14: 24590630 | 2- | 3 | 2 | G435R | |
| | | | Generation | | | | |
| G/A | RNF31 | chr14: 24617687 | Extended | 9 | 1 | splicing | |
| G/C | IRF9 | chr14: 24634003 | Extended | 9 | 1 | R277T | |
| G/A | SDR39U1 | chr14: 24909513 | 2- | 6 | 2 | P220S | |
| | | | Generation | | | | |
| T/A | PRKD1 | chr14: 30095731 | 2- | 3 | 2 | D586V | |
| | | | Generation | | | | |
| C/T | SEC23A | chr14: 39545251 | 2- | 3 | 1 | G292D | |
| | | | Generation | | | | |
| G/A | ITPK1 | chr14: 93418316 | 2- | 5 | 2 | P238L | |
| | | | Generation | | | | |
| G/A | CCDC85C | chr14: 99988547 | Extended | 9 | 1 | R300W | |
| A/G | C14orf2 | chr14: 104381450 | 2- | 6 | 5 | I26T | |
| | | | Generation | | | | |
| G/T | TRPM1 | chr15: 31329966 | Extended | 5 | 1 | T857K | |
| T/C | FMN1 | chr15: 33359761 | Extended | 9 | 3 | R109G | |
| G/T | PGBD4 | chr15: 34395847 | Extended | 9 | 2 | G372V | 0.000231 |
| C/T | JMJD7 | chr15: 42129054 | Extended | 9 | 4 | R260C | 0.00068 |
| C/T | CASC4 | chr15: 44620915 | Extended | 5 | 1 | R139* | |
| G/C | SPATA5L1 | chr15: 45695534 | 2- | 5 | 3 | D303H | |
| | | | Generation | | | | |
| C/G | PYGO1 | chr15: 55839207 | Extended | 7 | 1 | G92R | |
| C/G | PRTG | chr15: 55916638 | Extended | 9 | 2 | A999P | |
| G/A | NUDT7 | chr16: 77756514 | Extended | 9 | 3 | R12K, | |
| | | | | | | splicing | |
| G/A | DEFB124 | chr20: 30053379 | Extended | 7 | 4 | P49L | 0.000154 |
| A/G | EPB41L1 | chr20: 34809850 | Extended | 9 | 1 | D733G | |

*Indicates a mutation that results in a nonsense codon. Standard single letter amino acid designations are used.

TABLE 8
Biological functions/pathways of genes with variants found in children with ASDs

| Function | Gene names | References |
|---|---|---|
| Previously associated with autism | TRPM1, RAB11FIP5, AKAP9, SCN3A | 27, 43, 49, 72 |
| Previously associated with | RELN (autosomal recessive lissencephaly), ALX1 | 44-45, 50, 73, 74, 75 |
| neurological | (facial clefting, micropthalmia), CCDC85C | |
| disorder (other than autism) | (seizures), EPB41L1 (intellectual disability) | |
| Neural function | ITPK1, CLMN, PRTG | 76, 77, 78 |
| Mitochondrial function | PLA2G4B, c7orf10, PDK4, C14orf2 | 79, 80, 81, 82 |
| Inflammatory response/Immune | DEFB124, BPI, RNF31, IRF9 | 83, 84, 85, 86 |
| function | | |

TABLE 9
Summary of 30 Utah ASP families

| | Number of total | Number of genotyped |
|---|---|---|
| Generations | genotyped subjects* | ASD subjects* |
| 6 | 32 | 7 |
| 9 | 40 | 10 |
| 8 | 42 | 10 |
| 8 | 8 | 4 |
| 7 | 27 | 9 |
| 8 | 9 | 3 |
| 8 | 20 | 7 |
| 8 | 20 | 7 |
| 8 | 26 | 7 |
| 8 | 26 | 7 |
| 8 | 19 | 4 |
| 8 | 12 | 5 |
| 9 | 8 | 2 |
| 8 | 11 | 4 |
| 7 | 6 | 2 |
| 8 | 6 | 2 |
| 9 | 8 | 3 |
| 7 | 7 | 3 |
| 8 | 11 | 3 |
| 8 | 11 | 3 |
| 4 | 16 | 4 |
| 3 | 10 | 5 |
| 8 | 26 | 6 |
| 3 | 14 | 6 |
| 4 | 22 | 9 |
| 3 | 7 | 2 |
| 2 | 7 | 5 |
| 2 | 6 | 3 |
| 2 | 5 | 3 |
| 2 | 8 | 6 |

*Note that some individuals overlap between families, so the total number of individuals genotyped is less that the total numbers in this table.

TABLE 10

| 23 Genes Of Interest From | | | | |
|---|---|---|---|---|
| Literature, Located Outside Of | | | | |
| Shared Haplotype Regions | Chr | Location (hg18) | Location (hg19) | References |
| NOTCH2 | 1 | 120251699-120417799 | 120450176-120616276 | Garbett et al., 2008 |
| NRXN1 | 2 | 49996992-51117178 | 50143488-51263674 | Sutcliffe 2008, Morrow et al., 2008 |
| CNTN3 | 3 | 74390412-74657033 | 74307722-74574343 | Sutcliffe 2008, Morrow et al., 2008 |
| NHE9 (SLC9A9) | 3 | 144462754-145053979 | 142980064-143571289 | Sutcliffe 2008, Morrow et al., 2008 |
| DIA1 (c3orf58) | 3 | 145169603-145197895 | 143686913-143715205 | Sutcliffe 2008, Morrow et al., 2008 |
| PCDH7 | 4 | 30327135-30761519 | 30718037-31152421 | Yoshida et al., 1999 |
| PCDH10 | 4 | 134285920-134336182 | 134066470-134116732 | Sutcliffe 2008 |
| RNF8 | 6 | 37425726-37474492 | 37317748-37366514 | Sutcliffe 2008 |
| MAGI2 | 7 | 77480310-78924826 | 77642374-79086890 | Iida et al., 2004 |
| MET | 7 | 116095695-116229676 | 116308459-116442440 | Sutcliffe 2008 |
| EN2 | 7 | 154939585-154954287 | 155246824-155261526 | Sutcliffe 2008 |
| GPHN | 14 | 66039878-66722278 | 66970125-67652525 | Fritschy et al., 2008 |
| Prader-Willi/Angelman (NIPA1) | 15 | 20590720-20642284 | 23039279-23090843 | Sahoo et al., 2006 |
| UBE3A | 15 | 23129489-23239221 | 25578396-25688128 | Sutcliffe 2008 |
| A2BP1 | 16 | 6005133-7706500 | 6065132-7766499 | Sutcliffe 2008 |
| SLC6A4 | 17 | 25545032-25590841 | 28520906-28566715 | Sutcliffe 2008 |
| SHANK3 | 22 | 49455936-49522507 | 51109070-51175641 | Sutcliffe 2008 |
| NLGN4X | X | 5814083-6160706 | 5804083-6150706 | Sutcliffe 2008 |
| NLGN3 | X | 70277436-70311776 | 70360711-70395051 | Sutcliffe 2008 |
| NHE6 (SLC9A6) | X | 134891252-134961094 | 135063586-135133428 | Sutcliffe 2008 |
| FMR1 | X | 146797201-146844333 | 146989509-147036641 | Sutcliffe 2008 |
| MECP2 | X | 152936458-153059772 | 153283264-153406578 | Sutcliffe 2008 |
| NLGN4Y | Y | 15140026-15468921 | 16630632-16959527 | Sutcliffe 2008 |

Garbett K, Ebert P J, Mitchell A, Lintas C, Manzi B, Mirnics K, Persico A M: Immune transcriptome alterations in the temporal cortex of subjects with autism. Neurobiol Dis. 2008 30(3): 303-311.
Sutcliffe J S: Genetics. Insights into the pathogenesis of autism. Science. 2008 321(5886): 208-209.
Morrow E M, Yoo S Y, Flavell S W, Kim T K, Lin Y, Hill R S, Mukaddes N M, Balkhy S, Gascon G, Hashmi A, Al-Saad S, Ware J, Joseph R M, Greenblatt R, Gleason D, Ertelt J A, Apse K A, Bodell A, Partlow J N, Barry B, Yao H, Markianos K, Ferland R J, Greenberg M E, Walsh C A: Identifying autism loci and genes by tracing recent shared ancestry. Science. 2008 321(5886): 218-223.
Yoshida K, Hida M, Watanabe M, Yamaguchi R, Tateyama S, Sugano S: cDNA cloning and chromosomal mapping of mouse BH-protocadherin. DNA Seq. 1999 10(1): 43-47.
Iida J, Hirabayashi S, Sato Y, Hata Y: Synaptic scaffolding molecule is involved in the synaptic clustering of neuroligin. Mol Cell Neurosci. 2004 27(4): 497-508.
Fritschy J M, Harvey R J, Schwarz G: Gephyrin: where do we stand, where do we go? Trends Neurosci. 2008 31(5): 257-264.
Gephyrin: where do we stand, where do we go? Trends Neurosci. 2008 31(5): 257-264.
Sahoo T, Peters S U, Madduri N S, Glaze D G, German J R, Bird L M, Barbieri-Welge R, Bichell T J, Beaudet A L, Bacino C A: Microarray based comparative genomic hybridization testing in deletion bearing patients with Angelman syndrome: genotype-phenotype correlations. J Med Genet. 2006 43(6): 512-516.

TABLE 11

| Location | Location | Reference | Variant |
|---|---|---|---|
| (hg18, NCBI Build 36) | (hg19, NCBI Build 37) | Allele | Allele |
| chr1: 1878053 | chr1: 1888193 | C | A |
| chr1: 74809371 | chr1: 75036783 | T | C |
| chr1: 120239407 | chr1: 120437884 | A | G |
| chr1: 143623510 | chr1: 144912153 | A | G |
| chr1: 178125067 | chr1: 179858444 | G | A |
| chr2: 50054614 | chr2: 50201110 | A | G |
| chr2: 53809354 | chr2: 53955850 | C | T |
| chr2: 65979948 | chr2: 66126444 | G | T |
| chr2: 66649410 | chr2: 66795906 | T | C |
| chr2: 66652131 | chr2: 66798627 | T | C |
| chr2: 67485629 | chr2: 67632125 | C | T |
| chr2: 68238601 | chr2: 68385097 | A | G |
| chr2: 68903443 | chr2: 69049939 | G | T |
| chr2: 68903445 | chr2: 69049941 | T | C |
| chr2: 69030773 | chr2: 69177269 | C | A |
| chr2: 69504234 | chr2: 69650730 | G | A |
| chr2: 69512630 | chr2: 69659126 | A | T |
| chr2: 69588140 | chr2: 69734636 | G | A |
| chr2: 69623203 | chr2: 69769699 | G | A |
| chr2: 69887088 | chr2: 70033584 | C | T |
| chr2: 70042230 | chr2: 70188726 | G | A |
| chr2: 70341974 | chr2: 70488470 | C | T |
| chr2: 71016594 | chr2: 71163086 | T | C |
| chr2: 71016681 | chr2: 71163173 | C | T |
| chr2: 71065637 | chr2: 71212129 | A | T |
| chr2: 71190712 | chr2: 71337204 | G | A |
| chr2: 73156164 | chr2: 73302656 | G | A |
| chr2: 73345090 | chr2: 73491582 | C | A |
| chr2: 73489288 | chr2: 73635780 | C | T |
| chr2: 73505475 | chr2: 73651967 | C | T |
| chr2: 73529177 | chr2: 73675669 | T | G |
| chr2: 73533374 | chr2: 73679866 | T | C |
| chr2: 73533498 | chr2: 73679990 | T | A |
| chr2: 73534016 | chr2: 73680508 | G | C |
| chr2: 73570611 | chr2: 73717103 | G | C |
| chr2: 73571075 | chr2: 73717567 | G | T |
| chr2: 73721750 | chr2: 73868242 | C | A |
| chr2: 73860644 | chr2: 74007136 | T | C |
| chr2: 74127837 | chr2: 74274329 | C | T |
| chr2: 74541990 | chr2: 74688482 | G | A |
| chr2: 74543547 | chr2: 74690039 | G | A |
| chr2: 74578686 | chr2: 74725178 | G | A |
| chr2: 98294926 | chr2: 98928494 | G | A |
| chr2: 154973869 | chr2: 155265623 | G | A |
| chr2: 158666851 | chr2: 158958605 | G | T |
| chr2: 159371845 | chr2: 159663599 | T | C |
| chr2: 159662421 | chr2: 159954175 | C | T |
| chr2: 159750603 | chr2: 160042357 | C | A |
| chr2: 159821127 | chr2: 160112881 | G | T |
| chr2: 160003025 | chr2: 160294779 | T | C |
| chr2: 160003088 | chr2: 160294842 | A | G |
| chr2: 160018492 | chr2: 160310246 | A | G |
| chr2: 160312625 | chr2: 160604379 | C | T |
| chr2: 160312760 | chr2: 160604514 | C | T |
| chr2: 160381765 | chr2: 160673519 | G | A |
| chr2: 160398902 | chr2: 160690656 | G | A |
| chr2: 160419291 | chr2: 160711045 | G | C |
| chr2: 160451286 | chr2: 160743040 | T | A |
| chr2: 160512176 | chr2: 160803930 | C | A |
| chr2: 160548830 | chr2: 160840584 | C | A |
| chr2: 166245450 | chr2: 166537204 | A | T |
| chr2: 166482066 | chr2: 166773820 | G | A |
| chr2: 166600847 | chr2: 166892601 | G | A |
| chr2: 166807404 | chr2: 167099158 | A | G |
| chr2: 166814099 | chr2: 167105853 | C | G |
| chr2: 166970415 | chr2: 167262169 | T | C |
| chr2: 167823571 | chr2: 168115325 | A | G |
| chr2: 167823956 | chr2: 168115710 | T | G |
| chr2: 167824043 | chr2: 168115797 | G | C |
| chr2: 169415674 | chr2: 169707428 | C | T |
| chr2: 169429623 | chr2: 169721377 | G | A |
| chr2: 169472792 | chr2: 169764546 | C | G |
| chr2: 169805953 | chr2: 170097707 | T | G |
| chr2: 169837793 | chr2: 170129547 | C | T |
| chr2: 169855748 | chr2: 170147502 | C | G |
| chr2: 170075397 | chr2: 170367151 | T | G |
| chr2: 170259378 | chr2: 170551132 | G | A |
| chr2: 170779228 | chr2: 171070982 | G | A |
| chr2: 170952065 | chr2: 171243819 | G | A |
| chr2: 171084214 | chr2: 171375968 | C | T |
| chr2: 171108695 | chr2: 171400449 | T | C |
| chr2: 171530822 | chr2: 171822576 | C | T |
| chr2: 171624741 | chr2: 171916495 | C | A |
| chr2: 171904311 | chr2: 172196065 | C | A |
| chr2: 173038614 | chr2: 173330368 | C | T |
| chr2: 179351898 | chr2: 179643653 | G | T |
| chr2: 231477475 | chr2: 231769231 | T | C |
| chr2: 231483338 | chr2: 231775094 | C | A |
| chr2: 231573388 | chr2: 231865144 | G | C |
| chr2: 231864328 | chr2: 232156084 | C | T |
| chr2: 232087036 | chr2: 232378792 | C | T |
| chr2: 232166687 | chr2: 232458443 | T | C |
| chr2: 233341704 | chr2: 233633460 | G | A |
| chr2: 233543219 | chr2: 233834975 | A | G |
| chr2: 234050873 | chr2: 234386134 | A | G |
| chr2: 234059226 | chr2: 234394487 | G | A |
| chr2: 234059308 | chr2: 234394569 | A | G |
| chr2: 234096756 | chr2: 234432017 | A | G |
| chr2: 234266941 | chr2: 234602202 | A | C |
| chr2: 234413997 | chr2: 234749258 | T | C |
| chr2: 234414093 | chr2: 234749354 | G | A |
| chr2: 234414519 | chr2: 234749780 | G | C |
| chr2: 234415281 | chr2: 234750542 | G | C |
| chr2: 234415570 | chr2: 234750831 | T | C |
| chr2: 234519279 | chr2: 234854540 | G | C |
| chr2: 234519291 | chr2: 234854552 | A | G |
| chr2: 234643397 | chr2: 234978658 | C | T |
| chr2: 235614616 | chr2: 235949877 | T | C |
| chr2: 236372905 | chr2: 236708166 | C | T |
| chr2: 237070852 | chr2: 237406113 | C | T |
| chr2: 237153919 | chr2: 237489180 | C | A |
| chr2: 237908031 | chr2: 238243292 | G | A |
| chr2: 237909702 | chr2: 238244963 | A | G |
| chr2: 237912473 | chr2: 238247734 | C | G |
| chr2: 237940549 | chr2: 238275810 | C | A |
| chr2: 238091881 | chr2: 238427142 | T | C |
| chr2: 238091933 | chr2: 238427194 | T | C |
| chr2: 238099173 | chr2: 238434434 | C | T |
| chr2: 238307199 | chr2: 238642460 | G | T |
| chr2: 240630048 | chr2: 240981375 | T | A |
| chr3: 44923483 | chr3: 44948479 | C | T |
| chr3: 74417148 | chr3: 74334458 | G | A |
| chr3: 144853891 | chr3: 143371201 | C | T |
| chr3: 176434450 | chr3: 174951756 | T | C |
| chr3: 176647773 | chr3: 175165079 | C | T |
| chr3: 176955741 | chr3: 175473047 | T | C |
| chr3: 180445045 | chr3: 178962351 | T | A |
| chr3: 180805079 | chr3: 179322385 | A | C |
| chr3: 184237903 | chr3: 182755209 | T | G |
| chr3: 184416451 | chr3: 182933757 | C | A |
| chr3: 185150325 | chr3: 183667631 | G | A |
| chr3: 185153751 | chr3: 183671057 | C | A |
| chr3: 185182210 | chr3: 183699516 | T | C |
| chr3: 185235658 | chr3: 183752964 | A | C |
| chr3: 185236972 | chr3: 183754278 | C | G |
| chr3: 185382526 | chr3: 183899832 | C | T |
| chr3: 185526179 | chr3: 184043485 | T | C |
| chr4: 24590787 | chr4: 24981689 | A | T |
| chr4: 24972999 | chr4: 25363901 | T | A |
| chr4: 139188984 | chr4: 138969534 | T | C |
| chr4: 140860153 | chr4: 140640703 | G | T |
| chr4: 141274820 | chr4: 141055370 | C | A |
| chr4: 141536518 | chr4: 141317068 | G | A |
| chr4: 141539531 | chr4: 141320081 | G | A |
| chr6: 10810785 | chr6: 10702799 | G | A |
| chr6: 29515934 | chr6: 29407955 | C | T |
| chr7: 8234803 | chr7: 8268278 | C | A |
| chr7: 11488062 | chr7: 11521537 | G | A |
| chr7: 11547724 | chr7: 11581199 | C | A |
| chr7: 36293842 | chr7: 36327317 | C | T |
| chr7: 36884209 | chr7: 36917684 | C | A |
| chr7: 37873829 | chr7: 37907304 | T | C |
| chr7: 37913689 | chr7: 37947164 | G | T |
| chr7: 38323363 | chr7: 38356838 | G | T |
| chr7: 38400251 | chr7: 38433726 | T | G |
| chr7: 38435564 | chr7: 38469039 | C | A |
| chr7: 40465321 | chr7: 40498796 | C | T |
| chr7: 89776616 | chr7: 89938680 | C | T |
| chr7: 91440992 | chr7: 91603056 | C | T |
| chr7: 91552847 | chr7: 91714911 | C | T |
| chr7: 91552873 | chr7: 91714937 | C | A |
| chr7: 91562391 | chr7: 91724455 | C | T |
| chr7: 92571911 | chr7: 92733975 | G | A |
| chr7: 92572919 | chr7: 92734983 | A | G |
| chr7: 92573090 | chr7: 92735154 | G | A |
| chr7: 92663124 | chr7: 92825188 | C | T |
| chr7: 92893689 | chr7: 93055753 | A | G |
| chr7: 92908747 | chr7: 93070811 | C | T |
| chr7: 92954235 | chr7: 93116299 | A | G |
| chr7: 93354564 | chr7: 93516628 | T | C |
| chr7: 93879331 | chr7: 94041395 | C | A |
| chr7: 94132618 | chr7: 94294682 | C | A |
| chr7: 94132918 | chr7: 94294982 | C | T |
| chr7: 95638773 | chr7: 95800837 | C | A |
| chr7: 96488152 | chr7: 96650216 | G | T |
| chr7: 97326505 | chr7: 97488569 | A | T |
| chr7: 97659791 | chr7: 97821855 | T | C |
| chr7: 97690335 | chr7: 97852399 | G | A |
| chr7: 98283065 | chr7: 98445129 | G | C |
| chr7: 98716480 | chr7: 98878544 | C | T |
| chr7: 98870453 | chr7: 99032517 | G | A |
| chr7: 98883831 | chr7: 99045895 | C | A |
| chr7: 98923039 | chr7: 99085103 | T | C |
| chr7: 99108475 | chr7: 99270539 | C | T |
| chr7: 99285177 | chr7: 99447241 | T | C |
| chr7: 99295541 | chr7: 99457605 | C | G |
| chr7: 99312363 | chr7: 99474427 | A | G |
| chr7: 99327804 | chr7: 99489868 | G | C |
| chr7: 99507738 | chr7: 99669802 | A | G |
| chr7: 99526888 | chr7: 99688952 | G | A |
| chr7: 99557938 | chr7: 99720002 | G | T |
| chr7: 100036322 | chr7: 100198386 | C | T |
| chr7: 100172503 | chr7: 100334567 | C | A |
| chr7: 100186381 | chr7: 100348445 | C | T |
| chr7: 100188699 | chr7: 100350763 | T | G |
| chr7: 100193821 | chr7: 100355885 | C | T |
| chr7: 100203549 | chr7: 100365613 | G | T |
| chr7: 100204220 | chr7: 100366284 | T | C |
| chr7: 100209036 | chr7: 100371100 | C | A |
| chr7: 100209050 | chr7: 100371114 | C | T |
| chr7: 100209410 | chr7: 100371474 | G | A |
| chr7: 100224836 | chr7: 100386900 | T | C |
| chr7: 100324221 | chr7: 100486285 | G | T |
| chr7: 100390486 | chr7: 100552550 | T | C |
| chr7: 100390611 | chr7: 100552675 | C | T |
| chr7: 100462232 | chr7: 100675512 | G | A |
| chr7: 100468079 | chr7: 100681359 | C | G |
| chr7: 100468481 | chr7: 100681761 | C | T |
| chr7: 100604621 | chr7: 100817901 | A | G |
| chr7: 100626011 | chr7: 100839291 | T | A |
| chr7: 100981144 | chr7: 101194424 | C | T |
| chr7: 101708055 | chr7: 101921335 | G | A |
| chr7: 103021438 | chr7: 103234202 | C | T |
| chr7: 104570102 | chr7: 104782866 | C | A |
| chr7: 104935919 | chr7: 105148683 | A | G |
| chr7: 104964277 | chr7: 105177041 | A | T |
| chr7: 105445687 | chr7: 105658451 | G | A |
| chr7: 105448208 | chr7: 105660972 | C | T |
| chr7: 105458503 | chr7: 105671267 | T | C |
| chr7: 105525512 | chr7: 105738276 | C | A |
| chr7: 107214558 | chr7: 107427322 | A | C |
| chr7: 107408366 | chr7: 107621130 | C | A |
| chr7: 107507398 | chr7: 107720162 | C | A |
| chr7: 107588172 | chr7: 107800936 | C | T |
| chr7: 107621849 | chr7: 107834613 | G | C |
| chr7: 107653325 | chr7: 107866089 | C | A |
| chr7: 116199159 | chr7: 116411923 | C | T |
| chr7: 142753362 | chr7: 143043240 | C | T |
| chr7: 142790281 | chr7: 143080159 | C | A |
| chr7: 142798989 | chr7: 143088867 | T | C |
| chr7: 142805594 | chr7: 143095472 | C | A |
| chr7: 142885467 | chr7: 143175345 | C | T |
| chr7: 143332449 | chr7: 143701516 | C | T |
| chr7: 143402870 | chr7: 143771937 | G | C |
| chr7: 143438237 | chr7: 143807304 | T | C |
| chr7: 144064280 | chr7: 144433347 | C | T |
| chr7: 147774021 | chr7: 148143088 | G | C |
| chr7: 148764849 | chr7: 149133916 | C | T |
| chr7: 148783839 | chr7: 149152906 | G | A |
| chr7: 149107052 | chr7: 149476119 | G | T |
| chr7: 149112008 | chr7: 149481075 | C | A |
| chr7: 149112927 | chr7: 149481994 | G | T |
| chr7: 149113697 | chr7: 149482764 | C | T |
| chr7: 149115460 | chr7: 149484527 | T | C |
| chr7: 149116673 | chr7: 149485740 | C | T |
| chr7: 149133601 | chr7: 149502668 | C | A |
| chr7: 149134776 | chr7: 149503843 | T | G |
| chr7: 149137092 | chr7: 149506159 | C | A |
| chr7: 149144493 | chr7: 149513560 | T | C |
| chr7: 149146123 | chr7: 149515190 | A | — |
| chr7: 149146708 | chr7: 149515775 | C | G |
| chr7: 149146729 | chr7: 149515796 | C | T |
| chr7: 149148911 | chr7: 149517978 | G | T |
| chr7: 149149894 | chr7: 149518961 | T | C |
| chr7: 149153095 | chr7: 149522162 | G | T |
| chr7: 149153299 | chr7: 149522366 | T | G |
| chr7: 149154517 | chr7: 149523584 | C | T |
| chr7: 149805583 | chr7: 150174650 | T | C |
| chr7: 149848242 | chr7: 150217309 | C | T |
| chr7: 150122017 | chr7: 150491084 | T | G |
| chr7: 150131460 | chr7: 150500527 | C | T |
| chr7: 150161129 | chr7: 150530196 | T | G |
| chr7: 150185525 | chr7: 150554592 | G | C |
| chr7: 150188598 | chr7: 150557665 | C | G |
| chr7: 150363958 | chr7: 150733025 | T | A |
| chr7: 150378829 | chr7: 150747896 | C | T |
| chr7: 150392247 | chr7: 150761314 | G | A |
| chr7: 150504687 | chr7: 150873754 | G | A |
| chr7: 151135628 | chr7: 151504695 | C | T |
| chr8: 130830032 | chr8: 130760850 | A | G |
| chr9: 115122468 | chr9: 116082647 | C | G |
| chr9: 134772042 | chr9: 135782221 | T | C |
| chr11: 5321069 | chr11: 5364493 | T | C |
| chr11: 67198482 | chr11: 67441906 | A | C |
| chr12: 77066830 | chr12: 78542699 | T | C |
| chr12: 81276690 | chr12: 82752559 | T | G |
| chr12: 83801692 | chr12: 85277561 | T | A |
| chr12: 83962630 | chr12: 85438499 | G | A |
| chr12: 83973911 | chr12: 85449780 | C | A |
| chr12: 84042235 | chr12: 85518104 | T | C |
| chr12: 84198361 | chr12: 85674230 | G | T |
| chr12: 87004364 | chr12: 88480233 | C | G |
| chr12: 87425022 | chr12: 88900891 | C | A |
| chr14: 20622897 | chr14: 21553057 | T | C |
| chr14: 22953153 | chr14: 23883313 | T | G |
| chr14: 22956249 | chr14: 23886409 | G | C |
| chr14: 23062582 | chr14: 23992742 | T | G |
| chr14: 23072727 | chr14: 24002887 | G | T |
| chr14: 23073990 | chr14: 24004150 | A | G |
| chr14: 23104999 | chr14: 24035159 | G | A |
| chr14: 23105389 | chr14: 24035549 | G | A |
| chr14: 23596289 | chr14: 24526449 | A | G |
| chr14: 23604756 | chr14: 24534916 | G | T |
| chr14: 23633179 | chr14: 24563339 | A | G |
| chr14: 23671642 | chr14: 24601802 | C | A |
| chr14: 23675369 | chr14: 24605529 | A | G |
| chr14: 23684201 | chr14: 24614361 | T | G |
| chr14: 23749768 | chr14: 24679928 | G | A |
| chr14: 23798859 | chr14: 24729019 | T | G |
| chr14: 23830604 | chr14: 24760764 | G | A |
| chr14: 23876143 | chr14: 24806303 | G | A |
| chr14: 23876742 | chr14: 24806902 | G | A |
| chr14: 23906655 | chr14: 24836815 | C | G |
| chr14: 23971116 | chr14: 24901276 | G | T |
| chr14: 24145760 | chr14: 25075920 | G | A |
| chr14: 30860637 | chr14: 31790886 | T | G |
| chr14: 33338918 | chr14: 34269167 | G | C |
| chr14: 35859480 | chr14: 36789729 | T | G |
| chr14: 36751311 | chr14: 37681560 | G | T |
| chr14: 37343673 | chr14: 38273922 | T | G |
| chr14: 37347750 | chr14: 38277999 | C | T |
| chr14: 38786559 | chr14: 39716808 | T | C |
| chr14: 38791774 | chr14: 39722023 | G | C |
| chr14: 44044716 | chr14: 44974966 | G | A |
| chr14: 44044802 | chr14: 44975052 | A | G |
| chr14: 44045261 | chr14: 44975511 | G | A |
| chr14: 44674211 | chr14: 45604461 | C | T |
| chr14: 44676037 | chr14: 45606287 | C | T |
| chr14: 44735218 | chr14: 45665468 | C | G |
| chr14: 92482551 | chr14: 93412798 | T | A |
| chr14: 93458481 | chr14: 94388728 | C | T |
| chr14: 93500464 | chr14: 94430711 | G | A |
| chr14: 93826223 | chr14: 94756470 | C | A |
| chr14: 93917015 | chr14: 94847262 | T | A |
| chr14: 93982649 | chr14: 94912896 | T | G |
| chr14: 94003226 | chr14: 94933473 | C | A |
| chr14: 94003448 | chr14: 94933695 | G | A |
| chr14: 94005863 | chr14: 94936110 | G | A |
| chr14: 94176815 | chr14: 95107062 | G | A |
| chr14: 94669421 | chr14: 95599668 | G | A |
| chr14: 94749445 | chr14: 95679692 | G | C |
| chr14: 94976074 | chr14: 95906321 | G | A |
| chr14: 94982141 | chr14: 95912388 | G | A |
| chr14: 95226940 | chr14: 96157187 | G | A |
| chr14: 95773237 | chr14: 96703484 | C | T |
| chr14: 98252288 | chr14: 99182535 | C | T |
| chr14: 98710482 | chr14: 99640729 | C | A |
| chr14: 98712018 | chr14: 99642265 | G | A |
| chr14: 99047892 | chr14: 99978139 | G | A |
| chr14: 99450701 | chr14: 100380948 | T | C |
| chr14: 99685791 | chr14: 100616038 | G | A |
| chr14: 99861879 | chr14: 100792126 | C | T |
| chr14: 99864892 | chr14: 100795139 | G | T |
| chr14: 99865114 | chr14: 100795361 | C | A |
| chr14: 100268170 | chr14: 101198417 | A | G |
| chr14: 101088699 | chr14: 102018946 | A | G |
| chr14: 101088716 | chr14: 102018963 | G | C |
| chr14: 101372660 | chr14: 102302907 | T | C |
| chr14: 101799634 | chr14: 102729881 | A | G |
| chr14: 101799639 | chr14: 102729886 | G | A |
| chr14: 101819626 | chr14: 102749873 | G | A |
| chr14: 101985918 | chr14: 102916165 | C | T |
| chr14: 102043663 | chr14: 102973910 | C | T |
| chr14: 102045325 | chr14: 102975572 | C | T |
| chr14: 102411802 | chr14: 103342049 | T | C |
| chr14: 102439916 | chr14: 103370163 | C | T |
| chr14: 102504563 | chr14: 103434810 | G | A |
| chr14: 102636538 | chr14: 103566785 | C | T |
| chr14: 102638367 | chr14: 103568614 | T | A |
| chr14: 102941336 | chr14: 103871583 | A | C |
| chr14: 103243239 | chr14: 104173486 | G | A |
| chr14: 103249020 | chr14: 104179267 | T | C |
| chr14: 103251512 | chr14: 104181759 | A | C |
| chr14: 103269841 | chr14: 104200088 | G | A |
| chr15: 24767263 | chr15: 27184517 | G | A |
| chr15: 25933648 | chr15: 28260053 | G | A |
| chr15: 27208346 | chr15: 29421054 | C | T |
| chr15: 27799991 | chr15: 30012699 | G | A |
| chr15: 28880275 | chr15: 31092983 | A | G |
| chr15: 28984856 | chr15: 31197564 | G | A |
| chr15: 29142665 | chr15: 31355373 | C | A |
| chr15: 29156415 | chr15: 31369123 | A | G |
| chr15: 30797704 | chr15: 33010412 | G | A |
| chr15: 30878395- | chr15: 33091103- | CTT | — |
| 30878397 | 33091105 | | |
| chr15: 31144554 | chr15: 33357262 | A | G |
| chr15: 31146662 | chr15: 33359370 | C | T |
| chr15: 31146866 | chr15: 33359574 | C | A |
| chr15: 31233603 | chr15: 33446311 | G | C |
| chr15: 31659469 | chr15: 33872177 | C | T |
| chr15: 31741944 | chr15: 33954652 | C | T |
| chr15: 31803566 | chr15: 34016274 | G | A |
| chr15: 31829500 | chr15: 34042208 | A | G |
| chr15: 31867807 | chr15: 34080515 | C | T |
| chr15: 31924372- | chr15: 34137080- | AGA | — |
| 31924374 | 34137082 | | |
| chr15: 31947233 | chr15: 34159941 | T | G |
| chr15: 32309401- | chr15: 34522109- | CT | — |
| 32309402 | 34522110 | | |
| chr15: 32330427 | chr15: 34543135 | G | A |
| chr15: 32435104 | chr15: 34647812 | C | T |
| chr15: 32435939 | chr15: 34648647 | T | A |
| chr15: 32436227 | chr15: 34648935 | G | T |
| chr15: 32962108 | chr15: 35174816 | G | A |
| chr15: 33490388 | chr15: 35703096 | A | C |
| chr15: 34948333 | chr15: 37161041 | T | C |
| chr15: 37331804 | chr15: 39544512 | C | T |
| chr15: 37668777 | chr15: 39881485 | C | A |
| chr15: 37697723 | chr15: 39910431 | A | G |
| chr15: 38053091 | chr15: 40265799 | A | G |
| chr15: 38087546 | chr15: 40300254 | C | T |
| chr15: 38096151 | chr15: 40308859 | G | T |
| chr15: 38115086- | chr15: 40327794- | CTG | — |
| 38115088 | 40327796 | | |
| chr15: 38331785 | chr15: 40544493 | A | G |
| chr15: 38331812 | chr15: 40544520 | G | C |
| chr15: 38331909 | chr15: 40544617 | G | A |
| chr15: 38351868 | chr15: 40564576 | C | T |
| chr15: 38375863 | chr15: 40588571 | G | A |
| chr15: 38437727 | chr15: 40650435 | C | T |
| chr15: 38443137 | chr15: 40655845 | C | G |
| chr15: 38443165 | chr15: 40655873 | G | C |
| chr15: 38462447 | chr15: 40675155 | C | A |
| chr15: 38462735 | chr15: 40675443 | G | T |
| chr15: 38462785 | chr15: 40675493 | C | T |
| chr15: 38642502 | chr15: 40855210 | T | A |
| chr15: 38644281 | chr15: 40856989 | C | T |
| chr15: 38685935 | chr15: 40898643 | G | C |
| chr15: 38702482 | chr15: 40915190 | A | G |
| chr15: 38856063 | chr15: 41068771 | T | G |
| chr15: 38889458 | chr15: 41102166 | C | T |
| chr15: 39095657 | chr15: 41308365 | A | C |
| chr15: 39399166 | chr15: 41611874 | G | A |
| chr15: 39476458 | chr15: 41689166 | C | A |
| chr15: 39586617 | chr15: 41799325 | G | A |
| chr15: 39587003 | chr15: 41799711 | C | T |
| chr15: 39591046 | chr15: 41803754 | G | A |
| chr15: 39606659 | chr15: 41819367 | T | C |
| chr15: 39615049 | chr15: 41827757 | T | A |
| chr15: 39808804 | chr15: 42021512 | T | C |
| chr15: 39816112 | chr15: 42028820 | A | G |
| chr15: 39819675 | chr15: 42032383 | C | G |
| chr15: 39899045 | chr15: 42111753 | G | C |
| chr15: 39907634 | chr15: 42120342 | A | G |
| chr15: 39920587 | chr15: 42133295 | T | A |
| chr15: 39921389 | chr15: 42134097 | C | T |
| chr15: 39925171 | chr15: 42137879 | C | A |
| chr15: 39932384 | chr15: 42145092 | C | A |
| chr15: 39936888 | chr15: 42149596 | C | G |
| chr15: 39938180 | chr15: 42150888 | G | A |
| chr15: 39941669 | chr15: 42154377 | C | A |
| chr15: 39942030 | chr15: 42154738 | G | A |
| chr15: 39958829 | chr15: 42171537 | A | G |
| chr15: 39962630 | chr15: 42175338 | G | A |
| chr15: 39965414 | chr15: 42178122 | T | C |
| chr15: 39966894 | chr15: 42179602 | G | A |
| chr15: 39972867 | chr15: 42185575 | C | A |
| chr15: 40079445 | chr15: 42292153 | C | A |
| chr15: 40082164 | chr15: 42294872 | C | A |
| chr15: 40150370 | chr15: 42363078 | T | C |
| chr15: 40151383 | chr15: 42364091 | G | T |
| chr15: 40161102 | chr15: 42373810 | G | C |
| chr15: 40245287 | chr15: 42457995 | G | A |
| chr15: 40317035 | chr15: 42529743 | C | A |
| chr15: 40355839 | chr15: 42568547 | C | G |
| chr15: 40389913 | chr15: 42602621 | C | T |
| chr15: 40430821 | chr15: 42643529 | T | C |
| chr15: 40518548 | chr15: 42731256 | G | A |
| chr15: 40769632 | chr15: 42982340 | C | T |
| chr15: 40808275 | chr15: 43020983 | G | A |
| chr15: 40958085 | chr15: 43170793 | A | G |
| chr15: 41409390 | chr15: 43622098 | T | G |
| chr15: 41419841 | chr15: 43632549 | T | C |
| chr15: 41449094 | chr15: 43661802 | T | C |
| chr15: 41557143 | chr15: 43769851 | A | G |
| chr15: 41855277 | chr15: 44067985 | A | G |
| chr15: 41881219 | chr15: 44093927 | T | C |
| chr15: 42687962 | chr15: 44900670 | G | C |
| chr15: 42731049 | chr15: 44943757 | A | G |
| chr15: 42749480 | chr15: 44962188 | G | A |
| chr15: 43036413 | chr15: 45249121 | C | G |
| chr15: 43179367 | chr15: 45392075 | G | A |
| chr15: 43191358 | chr15: 45404066 | G | A |
| chr15: 43195706 | chr15: 45408414 | C | G |
| chr15: 43197024 | chr15: 45409732 | C | G |
| chr15: 43202449 | chr15: 45415157 | G | A |
| chr15: 43227892 | chr15: 45440600 | C | T |
| chr15: 43231425 | chr15: 45444133 | T | C |
| chr15: 43278374 | chr15: 45491082 | G | A |
| chr15: 43278428 | chr15: 45491136 | C | G |
| chr15: 43332770 | chr15: 45545478 | C | T |
| chr15: 43341559 | chr15: 45554267 | C | A |
| chr15: 43591939 | chr15: 45804647 | G | T |
| chr15: 43601625 | chr15: 45814333 | C | A |
| chr15: 43755727 | chr15: 45968435 | T | C |
| chr15: 53407088 | chr15: 55619796 | T | C |
| chr15: 53420151 | chr15: 55632859 | G | T |
| chr15: 53439957 | chr15: 55652665 | G | A |
| chr15: 53510164 | chr15: 55722872 | G | C |
| chr15: 53577202 | chr15: 55789910 | C | T |
| chr15: 53625877 | chr15: 55838585 | G | T |
| chr15: 53703995 | chr15: 55916703 | T | G |
| chr15: 53708336 | chr15: 55921044 | G | A |
| chr15: 53931921 | chr15: 56144629 | G | A |
| chr15: 53995755 | chr15: 56208463 | A | C |
| chr15: 54030903 | chr15: 56243611 | C | — |
| chr15: 54173160 | chr15: 56385868 | A | G |
| chr15: 54543577 | chr15: 56756285 | T | G |
| chr15: 55518865 | chr15: 57731573 | C | T |
| chr15: 56072564 | chr15: 58285272 | C | A |
| chr15: 57287408 | chr15: 59500116 | T | C |
| chr16: 73465703 | chr16: 74908202 | C | A |
| chr16: 74147593 | chr16: 75590092 | G | C |
| chr16: 74203924 | chr16: 75646423 | A | G |
| chr16: 75039502 | chr16: 76482001 | A | G |
| chr16: 75040248 | chr16: 76482747 | C | G |
| chr16: 75090084 | chr16: 76532583 | A | G |
| chr16: 75144703 | chr16: 76587202 | T | G |
| chr16: 75144832 | chr16: 76587331 | T | C |
| chr16: 75144850 | chr16: 76587349 | C | T |
| chr16: 75804018 | chr16: 77246517 | C | A |
| chr16: 75882826 | chr16: 77325325 | G | T |
| chr16: 76333173 | chr16: 77775672 | A | G |
| chr16: 77023938 | chr16: 78466437 | C | G |
| chr16: 77803188 | chr16: 79245687 | C | T |
| chr16: 77803321 | chr16: 79245820 | G | T |
| chr17: 69862619 | chr17: 72351024 | T | C |
| chr17: 71097920 | chr17: 73586325 | G | A |
| chr17: 77224886 | chr17: 79614481 | A | G |
| chr17: 77420095 | chr17: 79826806 | G | A |
| chr19: 50836865 | chr19: 46145025 | G | T |
| chr20: 7911041 | chr20: 7963041 | C | T |
| chr20: 7912476 | chr20: 7964476 | T | C |
| chr20: 8646451 | chr20: 8698451 | A | G |
| chr20: 8718822 | chr20: 8770822 | C | T |
| chr20: 9495018 | chr20: 9547018 | C | G |
| chr20: 25405022 | chr20: 25457022 | T | C |
| chr20: 29440610 | chr20: 29976949 | C | A |
| chr20: 29516983 | chr20: 30053322 | T | G |
| chr20: 30240997 | chr20: 30777336 | G | T |
| chr20: 30850110 | chr20: 31386449 | T | C |
| chr20: 31060133 | chr20: 31596472 | A | T |
| chr20: 31083161 | chr20: 31619500 | C | T |
| chr20: 31083176 | chr20: 31619515 | G | A |
| chr20: 31116257 | chr20: 31652596 | C | T |
| chr20: 31124204 | chr20: 31660543 | C | T |
| chr20: 31135260 | chr20: 31671599 | A | G |
| chr20: 31151921 | chr20: 31688260 | C | T |
| chr20: 32926503 | chr20: 33462842 | A | G |
| chr20: 33051846 | chr20: 33588185 | A | T |
| chr20: 33485887 | chr20: 34022473 | G | T |
| chr20: 33611412 | chr20: 34147998 | A | G |
| chr20: 33611736 | chr20: 34148322 | T | G |
| chr20: 33677587 | chr20: 34214173 | G | A |
| chr20: 34059785 | chr20: 34596371 | C | T |
| chr20: 34667606 | chr20: 35234192 | C | T |
| chr20: 34877296 | chr20: 35443882 | C | A |
| chr20: 34942544 | chr20: 35509130 | T | G |
| chr20: 35182837 | chr20: 35749423 | T | C |
| chr20: 35199751 | chr20: 35766337 | A | G |
| chr20: 36048999 | chr20: 36615585 | G | A |
| chr20: 36074389 | chr20: 36640975 | A | G |
| chr20: 36275328 | chr20: 36841914 | G | A |
| chr20: 36301520 | chr20: 36868106 | G | A |
| chr20: 36388138 | chr20: 36954724 | C | T |
| chr20: 36408359 | chr20: 36974945 | C | T |
| chr20: 36426747 | chr20: 36993333 | A | G |
| chr20: 37054466 | chr20: 37621052 | T | C |
| chr20: 37100596 | chr20: 37667182 | C | T |
| chr20: 39068030 | chr20: 39634616 | C | T |
| chr20: 39230879 | chr20: 39797465 | T | C |
| chr20: 39247143 | chr20: 39813729 | G | A |
| chr20: 39266184 | chr20: 39832770 | C | A |
| chr20: 39482993 | chr20: 40049579 | T | A |
| chr20: 40134806 | chr20: 40701392 | T | C |
| chr20: 40853311 | chr20: 41419897 | C | A |
| chr20: 49482271 | chr20: 50048864 | C | A |
| chr20: 49840909 | chr20: 50407502 | A | C |
| chr20: 50148404 | chr20: 50714997 | G | A |
| chr20: 51303743 | chr20: 51870336 | G | C |
| chr20: 51626044 | chr20: 52192637 | T | C |
| chr20: 51631553 | chr20: 52198146 | G | A |
| chr20: 51994876 | chr20: 52561469 | A | G |
| chr20: 52007378 | chr20: 52573971 | T | G |
| chr20: 54505879 | chr20: 55072472 | A | G |
| chr20: 55523287 | chr20: 56089881 | T | — |
| chr20: 55572027 | chr20: 56138621 | A | G |
| chr20: 56254533 | chr20: 56821127 | A | G |
| chr20: 56476086 | chr20: 57042680 | G | A |
| chr20: 56702274 | chr20: 57268867 | C | A |
| chr20: 56709564 | chr20: 57276157 | C | A |
| chr20: 56715597 | chr20: 57282190 | G | A |
| chr20: 56723754 | chr20: 57290347 | C | G |
| chr20: 56862842 | chr20: 57429447 | C | T |
| chr20: 56998090 | chr20: 57564695 | C | T |
| chr20: 57202002 | chr20: 57768607 | G | C |
| chr20: 57262696 | chr20: 57829301 | T | C |
| chr22: 49464446 | chr22: 51117580 | T | C |

TABLE 12
AAChange (UCSC KnownGenes)

uc010yrx.2:c.G441C:p.R147S
uc010zcy.2:c.A1148G:p.E383G
uc003flc.3:c.T103C:p.S35P
uc003flr.3:c.T460C:p.F154L
uc003tho.2:c.C862T:p.R288W
uc003ull.3:c.C385T:p.R129C
uc003ull.3:c.C2182T:p.R728C
uc003uml.3:c.G1192A:p.G398R
uc003unz.3:c.C506G:p.S169X
uc010liz.3:c.G4495C:p.D1499H
uc003whz.1:c.G1034C:p.R345P
uc001tae.4:c.G191T:p.R64L
uc001wkk.3:c.C295T:p.R99C
uc001wmc.3:c.G1003A:p.G335R
uc001wmq.3:c.G830C:p.R277T
uc001wpi.3:c.C334T:p.P112S
uc001wqh.3:c.A1757T:p.D586V
uc010tqc.1:c.G461A:p.G154D
uc001ybf.3:c.C356T:p.P119L
uc001yef.2:c.C472G:p.P158A
uc010avr.3:c.C898T:p.R300W
uc001ylm.3:c.C64T:p.Q22X
uc001yoi.4:c.T77C:p.I26T
uc010azy.3:c.C2174A:p.T725K
uc001zhf.4:c.A325G:p.R109G
uc001zho.3:c.G1115T:p.G372V
uc001znp.3:c.C494T:p.S165F
uc001zop.1:c.C478T:p.R160C
uc001zto.2:c.C415T:p.R139X
uc001zve.3:c.G907C:p.D303H
uc002adf.1:c.G274C:p.G92R
uc002adg.3:c.G2995C:p.A999P
uc002fff.3:c.G35A:p.R12K
uc002wvz.1:c.C146T:p.P49L
uc010gfq.3:c.A2798G:p.D933G

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

Patents, patent applications, patent application publications, journal articles and protocols referenced herein are incorporated by reference in their entireties, for all purposes.

1. A method for diagnosing a sample from a human subject as ASD-positive or ASD negative, comprising
detecting the presence of one or more single nucleotide polymorphism (SNP) classifier biomarkers in Table 1, Table 2, Table 3, Table 6 or Table 7 at the nucleic acid level by performing a polymerase chain reaction (PCR) with primers specific to the classifier biomarkers;
comparing the presence and/or absence of the one or more SNP classifier biomarkers of Table 1, Table 2, Table 3, Table 6 or Table 7 to the presence and/or absence of the of said SNP classifier biomarkers in at least one sample training set(s), wherein the at least one sample training set(s) comprise (i) data of the presence and/or absence of the one or more SNP classifier biomarkers of Table 1, Table 2, Table 3, Table 6 or Table 7 from an ASD positive sample or (ii) data of the presence and/or absence of the one or more SNP classifier biomarkers of Table 1, Table 2, Table 3, Table 6 or Table 7 from an ASD-negative sample,
and the comparing step comprises applying a statistical algorithm which comprises determining a correlation between the SNP classifier biomarker data obtained from the sample and the SNP classifier biomarker data from the at least one training set; and
diagnosing the sample as ASD positive or ASD negative based on the results of the statistical algorithm.

2. A method for classifying a sample from a human subject as a particular ASD subtype, comprising,
detecting the presence of one or more SNP classifier biomarkers in Table 1, Table 2, Table 3, Table 6 or Table 7 at the nucleic acid level by performing a polymerase chain reaction (PCR) with primers specific to the classifier biomarkers;
comparing the presence and/or absence of the one or more SNP classifier biomarkers of Table 1, Table 2, Table 3, Table 6 or Table 7 to the presence and/or absence of the of said SNP classifier biomarkers in at least one sample training set(s), wherein the at least one sample training set(s) comprise (i) data of the presence and/or absence of the one or more SNP classifier biomarkers of Table 1, Table 2, Table 3, Table 6 or Table 7 from a first ASD subtype positive sample or (ii) data of the presence and/or absence of the one or more SNP classifier biomarkers of Table 1, Table 2, Table 3, Table 6 or Table 7 from a second ASD subtype-positive sample,
and the comparing step comprises applying a statistical algorithm which comprises determining a correlation between the SNP classifier biomarker data obtained from the sample and the SNP classifier biomarker data from the at least one training set; and
diagnosing the sample as a particular ASD subtype based on the results of the statistical algorithm.

3. The method of claim 1, wherein the one or more SNP classifier biomarkers comprises two or more SNP classifier biomarkers, three or more SNP classifier biomarkers, four or more SNP classifier biomarkers, five or more SNP classifier biomarkers, six or more SNP classifier biomarkers, seven or more SNP classifier biomarkers, eight or more SNP classifier biomarkers, nine or more SNP classifier biomarkers, ten or more SNP classifier biomarkers, eleven or more SNP classifier biomarkers, twelve or more SNP classifier biomarkers, thirteen or more SNP classifier biomarkers, fourteen or more SNP classifier biomarkers, fifteen or more SNP classifier biomarkers, twenty or more SNP classifier biomarkers, twenty-five or more SNP classifier biomarkers, or thirty or more SNP classifier biomarkers.

4. The method of claim 1, wherein the hybridization assay is a microarray assay.

5. The method of claim 1, wherein the hybridization assay is a sequencing assay.

6. The method of claim 1, wherein the sample is from the human subject is a buccal sample.

7. The method of claim 1, further comprising identifying the human subject for ASD therapy based on the results of the statistical algorithm.

8. The method of claim 2, wherein the first ASD subtype and second ASD subtype are selected from the group consisting of Autistic disorder (classic autism), Asperger's disorder (Asperger syndrome), Pervasive developmental disorder not otherwise specified (PDD-NOS), and Childhood disintegrative disorder (CDD), wherein the first ASD subtype and second ASD subtype are different.

9. The method of claim 1, wherein the one or more SNP classifier biomarkers comprise SNPs in the RAB11FIP5, ABP1, and JMJD7-PLA2G4B genes.

10. The method of claim 9, wherein the RAB11FIP5 SNP is located at chr2: 73302656 (hg19), the ABP1 SNP is located at chr7:150554592 (hg19) and the JMJD7-PLA2G4B SNP is located at chr15:42133295 (hg19).

11. The method of claim 5, wherein the sequencing assay is a high throughput sequencing assay.

12. The method of claim 2, wherein the one or more SNP classifier biomarkers comprises two or more SNP classifier biomarkers, three or more SNP classifier biomarkers, four or more SNP classifier biomarkers, five or more SNP classifier biomarkers, six or more SNP classifier biomarkers, seven or more SNP classifier biomarkers, eight or more SNP classifier biomarkers, nine or more SNP classifier biomarkers, ten or more SNP classifier biomarkers, eleven or more SNP classifier biomarkers, twelve or more SNP classifier biomarkers, thirteen or more SNP classifier biomarkers, fourteen or more SNP classifier biomarkers, fifteen or more SNP classifier biomarkers, twenty or more SNP classifier biomarkers, twenty-five or more SNP classifier biomarkers, or thirty or more SNP classifier biomarkers.

13. The method of claim 2, wherein the hybridization assay is a microarray assay.

14. The method of claim 2, wherein the hybridization assay is a sequencing assay.

15. The method of claim 2, further comprising identifying the human subject for ASD therapy based on the results of the statistical algorithm.

16. The method of claim 2, wherein the one or more SNP classifier biomarkers comprise SNPs in the RAB11FIP5, ABP1, and JMJD7-PLA2G4B genes.

17. The method of claim 16, wherein the RAB11FIP5 SNP is located at chr2: 73302656 (hg19), the ABP1 SNP is located at chr7:150554592 (hg19) and the JMJD7-PLA2G4B SNP is located at chr15:42133295 (hg19).


SEQUENCE LISTING (78 sequences)

SEQ ID NO: 1 - DNA, 20 bp, Homo sapiens
gtgacaaggcaagacagacg

SEQ ID NO: 2 - DNA, 19 bp, Homo sapiens
tgagctcatcagcctgctc

SEQ ID NO: 3 - DNA, 20 bp, Homo sapiens
gtgacaaggcaagacagacg

SEQ ID NO: 4 - DNA, 19 bp, Homo sapiens
tgagctcatcagcctgctc

SEQ ID NO: 5 - DNA, 18 bp, Homo sapiens
ggcctcgctctcactcac

SEQ ID NO: 6 - DNA, 18 bp, Homo sapiens
ggactccgggatcacagt

SEQ ID NO: 7 - DNA, 21 bp, Homo sapiens
tcctccctttaattgcctctt

SEQ ID NO: 8 - DNA, 23 bp, Homo sapiens
caaccactttgaaacgtaaacaa

SEQ ID NO: 9 - DNA, 20 bp, Homo sapiens
gatgcagtttcgggaatgtt

SEQ ID NO: 10 - DNA, 21 bp, Homo sapiens
tggttctgaaagaggaactgg

SEQ ID NO: 11 - DNA, 20 bp, Homo sapiens
attccaacgcagttttctgg

SEQ ID NO: 12 - DNA, 20 bp, Homo sapiens
cctccttgtggactcaccat

SEQ ID NO: 13 - DNA, 21 bp, Homo sapiens
ccagcaaggaatgttcttgag

SEQ ID NO: 14 - DNA, 20 bp, Homo sapiens
tctctccaccagccagtttt

SEQ ID NO: 15 - DNA, 20 bp, Homo sapiens
tgggctttggagaaagagaa

SEQ ID NO: 16 - DNA, 23 bp, Homo sapiens
tgacattttagatggaggaaagc

SEQ ID NO: 17 - DNA, 20 bp, Homo sapiens
cttctggtgggctggagtta

SEQ ID NO: 18 - DNA, 20 bp, Homo sapiens
attccaggcaggttttctca

SEQ ID NO: 19 - DNA, 20 bp, Homo sapiens
cacactgcccagtgcttaaa

SEQ ID NO: 20 - DNA, 20 bp, Homo sapiens
atttcaggccatgaagatgc

SEQ ID NO: 21 - DNA, 20 bp, Homo sapiens
caccagtcatcagcctcaga

SEQ ID NO: 22 - DNA, 20 bp, Homo sapiens
aagtgcaaattatgccatgc

SEQ ID NO: 23 - DNA, 23 bp, Homo sapiens
cttgttacctgatattcctggtg

SEQ ID NO: 24 - DNA, 20 bp, Homo sapiens
aagctcagccctctgtggta

SEQ ID NO: 25 - DNA, 18 bp, Homo sapiens
gcaacgctgtgctctacg

SEQ ID NO: 26 - DNA, 20 bp, Homo sapiens
ggaaagtgtccaggaaggtg

SEQ ID NO: 27 - DNA, 20 bp, Homo sapiens
ggagacgctggacaatgagt

SEQ ID NO: 28 - DNA, 19 bp, Homo sapiens
ctagcgactcaccgctgct

SEQ ID NO: 29 - DNA, 19 bp, Homo sapiens
gtcggggaagtgaatggtg

SEQ ID NO: 30 - DNA, 20 bp, Homo sapiens
cgtcaccatggtaaggctgt

SEQ ID NO: 31 - DNA, 22 bp, Homo sapiens
ggtttactctgcatccctaccc

SEQ ID NO: 32 - DNA, 20 bp, Homo sapiens
cagtggagcagccactgtag

SEQ ID NO: 33 - DNA, 20 bp, Homo sapiens
cttgatggacttatgcacca

SEQ ID NO: 34 - DNA, 20 bp, Homo sapiens
acaaagccctccctctaagc

SEQ ID NO: 35 - DNA, 18 bp, Homo sapiens
gagcagcatggagcaggt

SEQ ID NO: 36 - DNA, 19 bp, Homo sapiens
ggttgctggccactaggat

SEQ ID NO: 37 - DNA, 20 bp, Homo sapiens
gtctgggcaaactcagcatt

SEQ ID NO: 38 - DNA, 20 bp, Homo sapiens
cttcccctggatacacatcg

SEQ ID NO: 39 - DNA, 24 bp, Homo sapiens
tgtttttcctgtaaatatcgcttt

SEQ ID NO: 40 - DNA, 22 bp, Homo sapiens
cattgggcttgtacctctagga

SEQ ID NO: 41 - DNA, 20 bp, Homo sapiens
atctccaaccaccattccag

SEQ ID NO: 42 - DNA, 27 bp, Homo sapiens
ttcatatgttttcttttaaactcttga

SEQ ID NO: 43 - DNA, 20 bp, Homo sapiens
ctaccctgctggagagcttg

SEQ ID NO: 44 - DNA, 20 bp, Homo sapiens
ccttcctgtcgctttttcag

SEQ ID NO: 45 - DNA, 20 bp, Homo sapiens
ggccttgatagccttcctct

SEQ ID NO: 46 - DNA, 20 bp, Homo sapiens
ggcaacctcagcagaaactc

SEQ ID NO: 47 - DNA, 20 bp, Homo sapiens
ctcacgttctgcagggagtc

SEQ ID NO: 48 - DNA, 19 bp, Homo sapiens
ccctccgtctaacccctct

SEQ ID NO: 49 - DNA, 22 bp, Homo sapiens
gctgcttcatttgtttacatgc

SEQ ID NO: 50 - DNA, 22 bp, Homo sapiens
aaagtttgctgtctggaagtga

SEQ ID NO: 51 - DNA, 23 bp, Homo sapiens
ttcctgacctcagaaaaatcaaa

SEQ ID NO: 52 - DNA, 20 bp, Homo sapiens
ccccatgaagccctactaca

SEQ ID NO: 53 - DNA, 22 bp, Homo sapiens
aagcccttgaagtttttcttga

SEQ ID NO: 54 - DNA, 20 bp, Homo sapiens
tgtgctgtgctctgttttcc

SEQ ID NO: 55 - DNA, 20 bp, Homo sapiens
cagaatcactggtggtgtgc

SEQ ID NO: 56 - DNA, 20 bp, Homo sapiens
acctgacctcggaaatgatg

SEQ ID NO: 57 - DNA, 20 bp, Homo sapiens
gactgatgcagttgggacag

SEQ ID NO: 58 - DNA, 20 bp, Homo sapiens
caacattgtcacctccttgc

SEQ ID NO: 59 - DNA, 26 bp, Homo sapiens
aatttatttgatggactttgtctcaa

SEQ ID NO: 60 - DNA, 21 bp, Homo sapiens
tctgtggttcttgtgggattc

SEQ ID NO: 61 - DNA, 20 bp, Homo sapiens
gggacagagcctgaagtcct

SEQ ID NO: 62 - DNA, 19 bp, Homo sapiens
acgtggtggaaccacagag

SEQ ID NO: 63 - DNA, 20 bp, Homo sapiens
tgcactccttctgacccttt

SEQ ID NO: 64 - DNA, 20 bp, Homo sapiens
agtgctgtccttcccacaag

SEQ ID NO: 65 - DNA, 23 bp, Homo sapiens
catcccatagcttctgaatagga

SEQ ID NO: 66 - DNA, 25 bp, Homo sapiens
ttcacaaggtaagtattgttcttcc

SEQ ID NO: 67 - DNA, 19 bp, Homo sapiens
ggagaccgaggagaacgtg

SEQ ID NO: 68 - DNA, 20 bp, Homo sapiens
gtcaacacctgggccactac

SEQ ID NO: 69 - DNA, 20 bp, Homo sapiens
atagcctccaaagccaggat

SEQ ID NO: 70 - DNA, 20 bp, Homo sapiens
caccaccgaatccaaactct

SEQ ID NO: 71 - DNA, 20 bp, Homo sapiens
gctccttccaggttctttcc

SEQ ID NO: 72 - DNA, 20 bp, Homo sapiens
tgataggccaggtggttcat

SEQ ID NO: 73 - DNA, 18 bp, Homo sapiens
ctttaggccgctcccaag

SEQ ID NO: 74 - DNA, 18 bp, Homo sapiens
gcctccgctacgatcaag

SEQ ID NO: 75 - DNA, 20 bp, Homo sapiens
ggacagcaggaaccagctac

SEQ ID NO: 76 - DNA, 20 bp, Homo sapiens
cctgccaaacttactgcaca

SEQ ID NO: 77 - DNA, 20 bp, Homo sapiens
gtgacctcacctccctctcc

SEQ ID NO: 78 - DNA, 20 bp, Homo sapiens
acagggtcagcaagaagtgg